SEQUENCE LISTING (91 sequences)

SEQ ID NO: 1 - DNA, 699 bp, unidentified
  source 1..699: mol_type = unassigned DNA
agctttaagaattcagggcctttttaacttgccaagccccacaccactccaggaacttccccacaccccagttctcagaattcatgtgcaaggtctttcctaaatccagggtccaggtcagagagtggaggatgtgctctatttcttacctgattgcagacccctctgacagtgctcccttctgaagcactcactgtctgaacgtacacagtctcagacttaatcatgcacagtgagcaagactgtggtgtgataattggcgtccctgacttattagggcaaatctatgggagggggagacctcctggaccactgagcaattaattcatttacattaggaagtttctccgtcagatgcaggaaaaaaatcttgttttcctgctgtggttttgacttttgccccatcttctgttgctgttgtaggaggcaaaataagggtcaaggcctggaaacacaagtgctttgactgaagctccacttggcttccgaagcccaagctgggttgtaccaggttccctagggtgcaggctgtgggcaactgccagggacatgtgcctgcccaccggcctctggccctcactgagttggccaatgggaaatgacaattgtgaggtggggactgcctgcccccgtgagtaccaggctgttgaggctgggccatctcctcctcacttccattctgactgcagtctgtggttc

SEQ ID NO: 2 - DNA, 1554 bp, unidentified
  source 1..1554: mol_type = unassigned DNA
atggccagcagaatcctgtggcacgagatgtggcacgagggcctggaggaggccagcagactgtacttcggcgagagaaacgtgaagggcatgttcgaggtgctggagcccctgcacgccatgatggagagaggcccccagaccctgaaggagaccagcttcaaccaggcctacggcagagacctgatggaggcccaggagtggtgcagaaagtacatgaagagcggcaacgtgaaggacctgctgcaggcctgggacctgtactaccacgtgttcagaagaatcagcaagctggagtacagcggcggcggcagcctggagggcgtgcaggtggagaccatcagccccggcgacggcagaaccttccccaagagaggccagacctgcgtggtgcactacaccggcatgctggaggacggcaagaagttcgacagcagcagagacagaaacaagcccttcaagttcatgctgggcaagcaggaggtgatcagaggctgggaggagggcgtggcccagatgagcgtgggccagagagccaagctgaccatcagccccgactacgcctacggcgccaccggccaccccggcatcatccccccccacgccaccctggtgttcgacgtggagctgctgaagctggagagcggcggcggcggcagcggcggcggcggcagcggcggcggcggcagcggcgtggacggcttcggcgacgtgggcgccctggagagcctgagaggcaacgccgacctggcctacatcctgagcatggagccctgcggccactgcctgatcatcaacaacgtgaacttctgcagagagagcggcctgagaaccagaaccggcagcaacatcgactgcgagaagctgagaagaagattcagcagcctgcacttcatggtggaggtgaagggcgacctgaccgccaagaagatggtgctggccctgctggagctggcccagcaggaccacggcgccctggactgctgcgtggtggtgatcctgagccacggctgccaggccagccacctgcagttccccggcgccgtgtacggcaccgacggctgccccgtgagcgtggagaagatcgtgaacatcttcaacggcaccagctgccccagcctgggcggcaagcccaagctgttcttcatccaggcctgcggcggcgagcagaaggaccacggcttcgaggtggccagcaccagccccgaggacgagagccccggcagcaaccccgagcccgacgccacccccttccaggagggcctgagaaccttcgaccagctggacgccatcagcagcctgcccacccccagcgacatcttcgtgagctacagcaccttccccggcttcgtgagctggagagaccccaagagcggcagctggtacgtggagaccctggacgacatcttcgagcagtgggcccacagcgaggacctgcagagcctgctgctgagagtggccaacgccgtgagcgtgaagggcatctacaagcagatgcccggctgcttcaacttcctgagaaagaagctgttcttcaagaccagcgccagctaa

SEQ ID NO: 3 - protein, 517 aa, unidentified
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLEYSGGGSLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGGSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 4 - protein, 401 aa, unidentified
MLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDTPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 5 - protein, 392 aa, unidentified
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLEYSGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 6 - protein, 650 aa, unidentified
MLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASQCTNYALLKLAGDVESNPGPGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGGSGGGGSMLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLES

SEQ ID NO: 7 - protein, 416 aa, unidentified [sequence omitted]

SEQ ID NO: 8 - protein, 281 aa, unidentified
GFGDVGALESLRGNADLAYILSMEPCGHCLIINNVNFCRESGLRTRTGSNIDCEKLRRRFSSLHFMVEVKGDLTAKKMVLALLELAQQDHGALDCCVWILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 9 - protein, 108 aa, unidentified
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 10 - protein, 100 aa, unidentified
MASRILWHEMWHEGLEEASRLYFGERNVKGMFEVLEPLHAMMERGPQTLKETSFNQAYGRDLMEAQEWCRKYMKSGNVKDLTQAWDLYYHVFRRISKLES

SEQ ID NO: 11 - protein, 100 aa, unidentified
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLES

SEQ ID NO: 12 - protein, 100 aa, unidentified
MASRILWHEMWHEGLEEASRLYFGERNVKGMFEVLEPLHAMMERGPQTLKETSFNQAYGRDLMEAQEWCRKYMKSGNVKDLHQAFDLYYHVFRRISKLES

SEQ ID NO: 13 - protein, 100 aa, unidentified
MASRILWHEMWHEGLEEASRLYFGERNVKGMFEVLEPLHAMMERGPQTLKETSFNQAYGRDLMEAQEWCRKYMKSGNVPDLTQAWDLYYHVFRRISKLES

SEQ ID NO: 14 - DNA, 1554 bp, unidentified
  source 1..1554: mol_type = unassigned DNA
atggcttctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagctcgagtatagcggcggcggcagcctggagggcgtgcaggtggagaccatcagcccaggcgacggcagaaccttccccaagagaggccagacctgcgtggtgcactataccggcatgctggaggacggcaagaagttcgacagcagccgcgaccgcaataagcccttcaagttcatgctgggcaagcaggaggtgatcagaggctgggaggagggcgtggcccagatgagcgtgggccagagagccaagctgaccatcagccccgactacgcctatggcgccaccggccaccccggcatcatcccaccccacgccaccctggtgtttgatgtggagctgctgaagctggagtccggcggaggcgggtctggaggaggcggcagcggcggcggcgggtcaggcgtggatggcttcggcgacgtgggagccctggagagcctgagaggcaacgccgatctggcctacatcctgagcatggagccctgtggccactgcctgatcatcaacaacgtgaacttctgccgggagagcggcctgcggacccggaccggcagcaacatcgactgcgagaagctgaggaggcgcttctcctccctgcactttatggtggaggtgaaaggcgatctgactgccaagaaaatggtgctggccctgctggagctggcccagcaggaccacggagccctggattgctgtgtggtggtgatcctgtcccacggctgccaggccagccacctgcagttccccggagccgtgtacggcaccgacggctgtcccgtgtccgtggagaagatcgtgaacatcttcaacggcacctcctgcccctccctgggcggcaagcccaagctgttctttatccaggcctgtggcggcgagcagaaggaccacggctttgaggtggccagcacctcccccgaggacgagagcccaggcagcaaccccgagcccgacgccacccccttccaggagggcctgcgcaccttcgaccagctggacgccatcagcagcctgcccacccccagcgacatcttcgtgagctacagcacctttcccggcttcgtgagctggcgcgatcccaagtccggctcttggtatgtggagaccctggacgacatctttgagcagtgggctcatagcgaggacctgcagagcctgctgctgcgcgtggccaatgccgtgagcgtgaagggcatctacaagcagatgccaggctgcttcaacttcctgcggaagaagctgttcttcaagaccagcgcctcctga

SEQ ID NO: 15 - DNA, 1209 bp, unidentified
  source 1..1209: mol_type = unassigned DNA
atgctggagggcgtgcaggtggagaccatcagcccaggcgacggcagaaccttccccaagagaggccagacctgcgtggtgcactataccggcatgctggaggacggcaagaagttcgacagcagccgcgaccgcaataagcccttcaagttcatgctgggcaagcaggaggtgatcagaggctgggaggagggcgtggcccagatgagcgtgggccagagagccaagctgaccatcagccccgactacgcctatggcgccaccggccaccccggcatcatcccaccccacgccaccctggtgtttgatgtggagctgctgaagctggagtccggaggcggctccggcgtggatggcttcggcgacgtgggagccctggagagcctgagaggcaacgccgatctggcctacatcctgagcatggagccctgtggccactgcctgatcatcaacaacgtgaacttctgccgggagagcggcctgcggacccggaccggcagcaacatcgactgcgagaagctgaggaggcgcttctcctccctgcactttatggtggaggtgaaaggcgatctgactgccaagaaaatggtgctggccctgctggagctggcccagcaggaccacggagccctggattgctgtgtggtggtgatcctgtcccacggctgccaggccagccacctgcagttccccggagccgtgtacggcaccgacggctgtcccgtgtccgtggagaagatcgtgaacatcttcaacggcacctcctgcccctccctgggcggcaagcccaagctgttctttatccaggcctgtggcggcgagcagaaggaccacggctttgaggtggccagcacctcccccgaggacgagagcccaggcagcaaccccgagcccgacgccacccccttccaggagggcctgcgcaccttcgaccagctggacgccatcagcagcctgcccacccccagcgacatcttcgtgagctacagcacctttcccggcttcgtgagctggcgcgatcccaagtccggctcttggtatgtggagaccctggacgacatctttgagcagtgggctcatagcgaggacctgcagagcctgctgctgcgcgtggccaatgccgtgagcgtgaagggcatctacaagcagatgccaggctgcttcaacttcctgcggaagaagctgttcttcaagaccagcgcctcctga

SEQ ID NO: 16 - DNA, 1179 bp, unidentified
  source 1..1179: mol_type = unassigned DNA
atggcttctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagctcgagtatagcggcggcggcagcggcgtggatggcttcggcgacgtgggagccctggagagcctgagaggcaacgccgatctggcctacatcctgagcatggagccctgtggccactgcctgatcatcaacaacgtgaacttctgccgggagagcggcctgcggacccggaccggcagcaacatcgactgcgagaagctgaggaggcgcttctcctccctgcactttatggtggaggtgaaaggcgatctgactgccaagaaaatggtgctggccctgctggagctggcccagcaggaccacggagccctggattgctgtgtggtggtgatcctgtcccacggctgccaggccagccacctgcagttccccggagccgtgtacggcaccgacggctgtcccgtgtccgtggagaagatcgtgaacatcttcaacggcacctcctgcccctccctgggcggcaagcccaagctgttctttatccaggcctgtggcggcgagcagaaggaccacggctttgaggtggccagcacctcccccgaggacgagagcccaggcagcaaccccgagcccgacgccacccccttccaggagggcctgcgcaccttcgaccagctggacgccatcagcagcctgcccacccccagcgacatcttcgtgagctacagcacctttcccggcttcgtgagctggcgcgatcccaagtccggctcttggtatgtggagaccctggacgacatctttgagcagtgggctcatagcgaggacctgcagagcctgctgctgcgcgtggccaatgccgtgagcgtgaagggcatctacaagcagatgccaggctgcttcaacttcctgcggaagaagctgttcttcaagaccagcgcctcctga

SEQ ID NO: 17 - DNA, 1956 bp, unidentified
  source 1..1956: mol_type = unassigned DNA
atgctggagggcgtgcaggtggagaccatcagcccaggcgacggcagaaccttccccaagagaggccagacctgcgtggtgcactataccggcatgctggaggacggcaagaagttcgacagcagccgcgaccgcaataagcccttcaagttcatgctgggcaagcaggaggtgatcagaggctgggaggagggcgtggcccagatgagcgtgggccagagagccaagctgaccatcagccccgactacgcctatggcgccaccggccaccccggcatcatcccaccccacgccaccctggtgtttgatgtggagctgctgaagctggagtccggaggcggctccggcgtggatggcttcggcgacgtgggagccctggagagcctgagaggcaacgccgatctggcctacatcctgagcatggagccctgtggccactgcctgatcatcaacaacgtgaacttctgccgggagagcggcctgcggacccggaccggcagcaacatcgactgcgagaagctgaggaggcgcttctcctccctgcactttatggtggaggtgaaaggcgatctgactgccaagaaaatggtgctggccctgctggagctggcccagcaggaccacggagccctggattgctgtgtggtggtgatcctgtcccacggctgccaggccagccacctgcagttccccggagccgtgtacggcaccgacggctgtcccgtgtccgtggagaagatcgtgaacatcttcaacggcacctcctgcccctccctgggcggcaagcccaagctgttctttatccaggcctgtggcggcgagcagaaggaccacggctttgaggtggccagcacctcccccgaggacgagagcccaggcagcaaccccgagcccgacgccacccccttccaggagggcctgcgcaccttcgaccagctggacgccatcagcagcctgcccacccccagcgacatcttcgtgagctacagcacctttcccggcttcgtgagctggcgcgatcccaagtccggctcttggtatgtggagaccctggacgacatctttgagcagtgggctcatagcgaggacctgcagagcctgctgctgcgcgtggccaatgccgtgagcgtgaagggcatctacaagcagatgccaggctgcttcaacttcctgcggaagaagctgttcttcaagaccagcgcctcccagtgcaccaattatgctttgcttaagctggcaggcgatgtggaatcaaacccgggtcctggggtacaggtggagaccatctctcctggcgacgggagaacatttcctaaaaggggccaaacatgcgtggttcactataccggtatgctggaggatggcaaaaaagtagactccagccgggatagaaacaaaccctttaagttcatgctgggtaagcaggaagttatacggggctgggaagagggagtagctcagatgtctgtgggccagagggccaagctgaccatctcaccggactacgcctacggcgctaccggccaccctggcattataccaccccatgcaactctcgtcttcgatgttgagttgctcaaactggaatcaggcggaggcgggtctggaggaggcggcagcatgctggagggcgtgcaggtggagaccatcagcccaggcgacggcagaaccttccccaagagaggccagacctgcgtggtgcactataccggcatgctggaggacggcaagaagttcgacagcagccgcgaccgcaataagcccttcaagttcatgctgggcaagcaggaggtgatcagaggctgggaggagggcgtggcccagatgagcgtgggccagagagccaagctgaccatcagccccgactacgcctatggcgccaccggccaccccggcatcatcccaccccacgccaccctggtgtttgatgtggagctgctgaagctggagtcctga

SEQ ID NO: 18 - protein, 125 aa, unidentified
MGSGPSNFVNHAPGEAIVTGLEKGADKVAGTISHTIWEVIAGLVALLTFLAFGFWLFKYLQKRRERRRQLTEFQKRYLRNSYRLSEIQRPISQHEYEDPYEPPSRRKPPPPPYSTYVNIDNVSAI

SEQ ID NO: 19 - protein, 154 aa, unidentified
MGSGPSNFVNHAPGEAIVTGLEKGADKVAGTISHTIWEVIAGLVALLTFLAFGFWLFKYLQWYNRKSKNKKRKEQIREQIELGLLSYGAGVASLPLLNVIAHNPGSVISATPIYKGPCTGVPNSRLLQITSGTAEENTRILNHDGRNPDGSINV

SEQ ID NO: 20 - protein, 38 aa, unidentified
MGSGPSNFVNHAPGEAIVTGLEKGADKVAGTISHTIWE

SEQ ID NO: 21 - protein, 48 aa, unidentified
KLLQWYNRKSKNKKRKEQIREQIELGLLSYGAGVASLPLLNVIAHNPG

SEQ ID NO: 22 - protein, 48 aa, unidentified
VISATPIYKGPCTGVPNSRLLQITSGTAEENTRILNHDGRNPDGSINV

SEQ ID NO: 23 - DNA, 228 bp, unidentified
  source 1..228: mol_type = unassigned DNA
ctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatgg

SEQ ID NO: 24 - DNA, 56 bp, unidentified
  source 1..56: mol_type = unassigned DNA
aataaaggaaatttattttcattgcaatagtgtgttggaattttttgtgtctctca

SEQ ID NO: 25 - protein, 15 aa, unidentified
GGGGSGGGGSGGGGS

SEQ ID NO: 26 - protein, 5 aa, unidentified
GGGGS

SEQ ID NO: 27 - protein, 4 aa, unidentified
GGGS

SEQ ID NO: 28 - skipped

SEQ ID NO: 29 - protein, 18 aa, unidentified
KESGSVSSEQLAQFRSLD

SEQ ID NO: 30 - protein, 14 aa, unidentified
EGKSSGSGSESKST

SEQ ID NO: 31 - protein, 12 aa, unidentified
GSAGSAAGSGEF

SEQ ID NO: 32 - protein, 5 aa, unidentified
EAAAK

SEQ ID NO: 33 - protein, 5 aa, unidentified
EAAAR

SEQ ID NO: 34 - protein, 5 aa, unidentified
PAPAP

SEQ ID NO: 35 - protein, 12 aa, unidentified
AEAAAKEAAAKA

SEQ ID NO: 36 - protein, 24 aa, unidentified
APVKQTLNFDLLKLAGDVESNPGP

SEQ ID NO: 37 - protein, 19 aa, unidentified
ATNFSLLKQAGDVEENPGP

SEQ ID NO: 38 - protein, 20 aa, unidentified
QCTNYALLKLAGDVESNPGP

SEQ ID NO: 39 - protein, 18 aa, unidentified
EGRGSLLTCGDVEENPGP

SEQ ID NO: 40 - protein, 27 aa, unidentified
GSGAPVKQTLNFDLLKLAGDVESNPGP

SEQ ID NO: 41 - protein, 22 aa, unidentified
GSGATNFSLLKQAGDVEENPGP

SEQ ID NO: 42 - protein, 23 aa, unidentified
GSGQCTNYALLKLAGDVESNPGP

SEQ ID NO: 43 - protein, 21 aa, unidentified
GSGEGRGSLLTCGDVEENPGP

SEQ ID NO: 44 - protein, 28 aa, unidentified
RRKRAPVKQTLNFDLLKLAGDVESNPGP

SEQ ID NO: 45 - protein, 23 aa, unidentified
RRKRATNFSLLKQAGDVEENPGP

SEQ ID NO: 46 - protein, 24 aa, unidentified
RRKRQCTNYALLKLAGDVESNPGP

SEQ ID NO: 47 - protein, 22 aa, unidentified
RRKREGRGSLLTCGDVEENPGP

SEQ ID NO: 48 - protein, 31 aa, unidentified
RRKRGSGAPVKQTLNFDLLKLAGDVESNPGP

SEQ ID NO: 49 - protein, 26 aa, unidentified
RRKRGSGATNFSLLKQAGDVEENPGP

SEQ ID NO: 50 - protein, 27 aa, unidentified
RRKRGSGQCTNYALLKLAGDVESNPGP

SEQ ID NO: 51 - protein, 25 aa, unidentified
RRKRGSGEGRGSLLTCGDVEENPGP

SEQ ID NO: 52 - protein, 413 aa, unidentified
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDYPYDVPDYALD

SEQ ID NO: 53 - protein, 400 aa, unidentified
MVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 54 - protein, 283 aa, unidentified
MGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPCIVSMLRKKLFFKTS

SEQ ID NO: 55 - protein, 413 aa, unidentified
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPCIVSMLRKKLFFKTSVDYPYDVPDYALD

SEQ ID NO: 56 - DNA, 1732 bp, unidentified
  source 1..1732: mol_type = unassigned DNA
aatgttttccaccccagagcctccacattcatgagggatgccaaaaatcgaagaagttcttcgtagatgtacagcccagcacattcacaactgagcattgttgcctgacaattcatttattcaacaatgaatgtttgagtgccatgcatggttttgggtatccacttgtgagcaagatgacctaggtccctgctctcacttggtggaggggtagggaaagcaggtaggtacggagaaggaaagaagatagaactctatgccctgcaagctactgctagaaaactaacacttctgtcttccctcgggactctcctaggccctttcaccacttctcacctctcacccacattctgcactcccagctaaagcagccaaaggactatttgatttggcagagctttcaggcacaaagccctatgttccctctggcccaaagccactactctgaaggctgaaaactgccctttggctcaggttgctgtccagtcacagcctcacagataatttgtgctgaaccttgaggtgagggcagaggccacttctttaaatactttgtcttctctactgccttacatttgaaaagcacaggttggatcaataagtattaaacgtatagcgtattactcctttgaagcctccctgcttgactcctccctttcctgccttctcattccctactttattgcatcacagatacgtaatttattaccaaggatctttcataattccccacccctgcctctttcttctcttgcccacactgtgtgacttaacagagtcaacaatacatataattccaccagcaacaaagaaaataagttttgttttcctattgtctgttttcatgaagaggcccttcttagttaatctgaaagctatggcttataccatagtcctccatcatatctaaagccaggaactagaacttgggcttgtgcttgagtcctgggtaaagaagggttgagtggtgctaattcttggctgtctgggtgctcttgattagtgatagaatcctagtcaacactgtcgcttatccagacccaggagaataaaaatctacttggctctcctactcttcattctggtagcatcttaggccaggtaaagttcaataaatttttttgaattctaagaagtgggaattcctccctcaactcaaaggctatattgtcaaaaactcaactcccggctctgtcgcttcttcactgagggcaatttatctccataatcaaagatgaatacattcagatgaactcttctgtgtgggtttaaattttactgttcagcattcagtcttgctagtaaataagaaaagatgagagaaatggattgaaaactcccacaagaccctctatggaaaaggagccaaaagattgcagaggtatatagaactttagagcttttgagtttaatgatgtctatccctataccttctttctggaaagcagatttttgtgaattgagggcaggaacattacagaactgatgcttcatggctctggaatactaagtcctctgagcctggatctccttcagctctgccacctacctccttgtttgctaaatgtccttagagaagttaggatatgcctggggaaccctgggtcatataaccatgacaactggctgaccctgttgtctctccctctctgccctcctctagctgggaggtggaagcagcattgcccaagcctcccaggagtgacaggaattgtttctgcctgaggagaca

SEQ ID NO: 57 - DNA, 1000 bp, unidentified
  source 1..1000: mol_type = unassigned DNA
tccctactttattgcatcacagatacgtaatttattaccaaggatctttcataattccccacccctgcctctttcttctcttgcccacactgtgtgacttaacagagtcaacaatacatataattccaccagcaacaaagaaaataagttttgttttcctattgtctgttttcatgaagaggcccttcttagttaatctgaaagctatggcttataccatagtcctccatcatatctaaagccaggaactagaacttgggcttgtgcttgagtcctgggtaaagaagggttgagtggtgctaattcttggctgtctgggtgctcttgattagtgatagaatcctagtcaacactgtcgcttatccagacccaggagaataaaaatctacttggctctcctactcttcattctggtagcatcttaggccaggtaaagttcaataaatttttttgaattctaagaagtgggaattcctccctcaactcaaaggctatattgtcaaaaactcaactcccggctctgtcgcttcttcactgagggcaatttatctccataatcaaagatgaatacattcagatgaactcttctgtgtgggtttaaattttactgttcagcattcagtcttgctagtaaataagaaaagatgagagaaatggattgaaaactcccacaagaccctctatggaaaaggagccaaaagattgcagaggtatatagaactttagagcttttgagtttaatgatgtctatccctataccttctttctggaaagcagatttttgtgaattgagggcaggaacattacagaactgatgcttcatggctctggaatactaagtcctctgagcctggatctccttcagctctgccacctacctccttgtttgctaaatgtccttagagaagttaggatatgcctggggaaccctgggtcatataaccatgacaactggctgaccctgttgtctctccctctctgccctcctctagctgggagg

SEQ ID NO: 58 - DNA, 308 bp, unidentified
  source 1..308: mol_type = unassigned DNA
cccctgcccctgcccctgcccccacccccacccccacccccacccccagccggcgcccgcgcccgcccccgcgccgggcccggctcggcccgacccggttccgccgcgggcaggcggggcccagcgcactcggagcccgagcccgagccgcagccgccgcctggggcgcttgggtcggcctcgaggacaccggagaggggcgccacgccgccgtggccggcagatttgaaagaagccgacactaaaccaccaatatacaacaaggccattttgtcaaacgagagtcagcctttaacgaaatgaccatg

SEQ ID NO: 59 - DNA, 597 bp, unidentified
  source 1..597: mol_type = unassigned DNA
cagtagcatgctgataccaacgtttaaactatggatacatatttgaattccaaatttttcttcagataatgtgattagagattagagattcaaccagggatagacaccgaaagaaaactttgcccaaataagctttctggtatttcataagcaagagatttaagttttccatttaagaagccattgtgaattatacaacaataaaaaatgcaagtggatattgaacagtctcttctctgataattctaaatacagtacagttcacgcccctcacgagacactgaacatgtggtcaccggcgagacagtgtggcaatattatccctgtaatgtaccaagtcttgccagagcagtgaacattatgacacaactttttgtcacagctggctcctaataggacagtgccagccaattcaagcccagtcctttctgtgtttattcccatctctcccaaatatttggaaactgatgtcttgactcatgggtgtattcacgattctgttacttcaagtctttttcttttaacggattgatcttttgctagatagagacaaatatcagtgtgaattacagcaaacccatattccatgctgttatg

SEQ ID NO: 60 - DNA, 1000 bp, unidentified
  source 1..1000: mol_type = unassigned DNA
gaggatcacttgagcccaggagtttcagactagtctaggcaacatgtcaagacacagtctctactaaacaattaaaattaaaaaaaaaaattagccaggcatggtggtgtgcacctgtagtcccagctacttgggagcctggggtgggaggattccttgagcccgggaagtcgaggctgaagtgagccgtgattgcgccacagcactccagcctgggcgacacagcaacaccctgtctcatggaagaaagaaagaaaagaaaggaagaaagaaaaaaaaaaagcagattggaactctggaattaacaagaagtaggacgcacggagcacttccgcctgagtggagactgtggatccgggtcaacctgactacctaaatcacaggccaataaatggtctttcagtggtcagtccctgtaagatccgtggctctcagcttcttatcttaggggctgtggaggaaggacatgattatgttgatttaagcgctgaatattttcccttgtgatacccatcctcgcaaaactttgcttcaaccacaaacgaggaccttctgtaccagaggggcaataaccacaatgaagctaggaagaaatgcagagcaccccagcatacagtccataagcttcctgaagtggggggcctcaggcatcgctgcctccccaaagaggatcaggcccagaacagtatgctccagaaataagactggaaaaagggaaagaggggcctcaagtccaggagaccagcggctttctgaacgcgcacctgccaacccactttggacaggtcacgatggacagcgtggcaggaaaagaaaaggtcactgtctacccaacacatgagaaactgtttctcgtgcctcacgtccccactccgtccccacccatgttgtctgagtccctcggtgtcagaaacactgctaagaaatttaagaaattctgttaatgagtttaagaaatgtttttaatgattaaaagtcagtgacttgtgaataaccatgt

SEQ ID NO: 61 - DNA, 1000 bp, unidentified
  source 1..1000: mol_type = unassigned DNA
cagtgggatacacagtgtagcgatgcatcactctgaaatattttagtttctttttttcccctaaatctgggtatgttcgtgggaatttgcagcacatgtgaacaacttctgtcattcttgcatgaggcaaagggaattgaaaaccacgattactttagaaaactagtttcacagattggtcactgtataaaagaaggatattggttttggtagcttgtgaccacacaccatttctgatctgaataaattcagaacttataatacagttcagaaattgaatgcagtttctcaatatgaggaaagtattttagaataaggcctatttttcaaaggatctgtggaaatcaatgctatgctctcatttaggagatggaaagagtgaggttaaattatcatttcgattaaatctacagtccagattactggtggatgaattgaatgtacttttttattcatataaaacatttgaaatcagaaatctggagtacttttaaatcccattatttattttgttttaatcgccaggtaattcctgagacaggagtgtcccgaagagcctttgcaattatgtaagaatctccgaggcagttcttatgttcctcaattcaaaagaaccacataactgcaatttaaataacaccccacacacacacacaaaataaggtcgaagtttatctcaaaataatttcccctctctacactgggataaatatgtataggaataatagggggaaattcagtgcactgagcattaagctgtcaaaacaggaatgtttaaaatatcctgttagtggtttaaaaataatttgtactctaagtccagtgactatttgccagggagaaccaaagttgagaaatttctattaaaaacatgactcagagaaaaaaatgcagaggccggtaatgaaggaaatgattggatctcattcccaattggtcattcctaagatcacatgttctgagcatctttaaaaggaagttatctggactcaagagggtc

SEQ ID NO: 62 - DNA, 5403 bp, unidentified
  source 1..5403: mol_type = unassigned DNA
aggagagccttggcatgatccttaacctatgacatacatgctgtgaaaacactccccctcatcagggctttgtccctactcctcatcatccagagagagggtatgtttcctcttcaacctaaaaattgtcagaaaaaatgctctcatttcctccaaaagaaataaacacaggcaaaggtcagagggatgcattccatggaagctgtccataggagagcaaagtgcaaggcagacggcctgagccagggctatgaagcactgagggtctggggctatgtccccagtctccacatccccaaagaaaagcaaaactctggctactgaagaccccttcccacagccctaaggaaacattctgacctggtgtgtgcatgtctatgctgtactggggtacgtgtgaataaaagtatagtcacctctgtgggaattcttttcatagtcaaaggcaaattgtctcctatgcaccttgtttctgagtactaagcccctccaaaagggaaggagtgtcctatgtactttctatattgtttctcaatatctttcactgctaccccattgtctcatttcattgcccttgcccagcccccatcatctcttaactcctgccttgtgagcagatttacatctgctcactctatcctttataatccactctgcctagggccactgtattcattgttctaaaactgaaataggagcccatggggggttttaaacacatatacaagcattaagcatgaatatgtagtttaaatccgtatcttattccttttgttaattatttattaattcaagtatttggtgaagaaccaggtgccaggaactttaagtcatgcatgggatagagtttgggatggagaagggaagtcaggaatgaggatagatcgggaatgggatgaatgtatctctcatcttgtatacattacatgctaatgaaaggtgataaaagtgtaaaaaaaattggaagagctactagtgccttctgtacaaaatatgctaatatcaagaagactatcaaaacaaaacaccttttaaaatgctttgtatgttattttggtcccaggctggtaactagaatgagagcatagattacaaaagagagacacaaattatgtcttatttattattatattcccttacccctatgtagtacagtgccttaagcataacaggcttgcaaaaaaagattttttgtaaattatttgccagcgtgcactttatacaatgaaactatgtttgaaaatgccaatagtataatttctactagttttcaagaaaggatgaccaccaaaattttggtgaagtccagattctcttcattaatgcttagtgaggatttccttcacagagcattgaggccaaggtcgtataatattgaattcgttttttcatgtgattcttcttattttgtattgcttcattcacatagcaatccatcaacattgtgatgaaagatgatagtaatttgatgagtgaacattccattttctccatgtcggcaagaataactcttaggtattaagataaaaataatttctttaggagctgaacagttgtttacattcctgtttatactgcagagcaggagttttcggcctctgcattcaatttctggacagcttatcatccagaaagagttctctaccctacacgagacaaaacctagtggacctttgcatagtaagaatattagtattccacaacatgttccatgtgtttaggatatacactgagctgcttttaagggcaatatctctcaggtaagaaactctgccaagccttgttactcttaacaagtgatcatatgaaaggtcttcctgtggactcaactaccttctcacatcttacttctctcattgctgtttaagttaccaagatcctttactataagttactactattcttgtttatatgaactctaaaagagccaatagttgtttctagaaaatctcaactacgccatttctccaacgatgatcaggcactgaatatgctggtatagaagatgaggctactggcctacaaatacattcttcaaggttttttttgttgttttttataggcgtgctggtggactctcactaacccgtcgaagaaggatttccttcaggcgttttactgaatatataattactttgtatctaacaatgaattaatctgaacaaaagtctcttccttaatggaccttggactttgctcaccaggaacatatatctgtatatgtcagggtggctgtgtttgggtgaaattgttctgaaaggtaatggaaccagcagctcccgcagttttctgtcccgtatacagtctctttcctgatcttcctgtggctgtcttcttctcatcattcagctaacagctcaattatctaccttctttaacagactttcccggattgccccatctagtgctccaaccctcacagtggtcattacatcagagtaccccattctgtttgctctaaaacacagcactacctgtgattggcttatttatttcatatttcatacacaaggtctctttgtatatgaccttctccagtattagaatataagctcattgagagcaagcaaatgcctgccctggctggctcactgttgaatccgcattggttagcacaatgcctgatcacatgttgaataaataagcgaagcttgctgttaccatggtactctaaaacttcactgcccaagagaaatataatgtaagccacatatataatctaaaattttctagcagccacataaggaaagtaaaaaagaaataggttaaattaattttcgtaatacattctatttaacctaacatatcgaaaatgttgtcatttcaacatgtaatcaatgtaagaaaatgaataatataggatatatttgtatattgtcttcaaaatctggtatatattttatactaacagttcatctcaattcagattctaaattttcttttgaaatacttgtccaacatttagatttcataaaatttacaattgaaaaagtagattgacatagccacattggttcaaacatatttaaaagtttttcagtaactgaattgaggatcatttctttcattttaattaaaattaagtaaaattaaaaattcaatttctcagtcatactagccacattttaagagccacatgtgaccggagattacactaccagagagtcaatctctaaaacaattattcacaataaaaaagtcacggaatcaacctaagtgtccatcagtggatgactggatgaagacaatgtgatatatatttaccatggaataccactcagccataaaaagggatgagatcatttcttttgcagcaacataggtgaaactggagaccattatcttaagtgaaataaatcagaaacagaaagtcaaataccacatattctcacttataagtgggagctaaacaatgtgtacacacagacgtacagagtggcataatagacaattggagactccaaaaggtgggaaggtgggaaggtgggagggggcaagggataagaaattacctatcagggccgggcacagtggctcacgcctgtaatcccagcactttgggagactgaggcgagtgggtcacctgaggtcaggagttcaagaccagcctggccaacatggcgaaaccctgtctctactaaaaatatgagaaaaaaaattatccaggcatgtggtgggcacctgtaatccccgctactagggaggctgaggtaggagaatcgcttgaacctgggaggcagaggttgcagtgagccaagatcgcgccactgcattccagcctggacaataaaatcaaaactccatctcaaaaaaaaaaaaaaaagaaagaaattaccaattgagtacaatatacactatttgggtgatgattacactaaaagtccagactttattacaactcaatatacatatgtaacaaaaatgcacatgtagtccgaaatctatagaaacaaaaattttaaaaaaccaaattgcagtgttagaatacacatagtatgtattcaaattcaatcatttaaaaacctgtaggaagcaaattaatttaaaacattatcaaaaacatccctctattaagatttgtcacttttatttactttccatctgaacagtcttattcaatgggaaacaggtggatcactaatagcaagcctacctggatggtacatcaccacagtctgtgtatctatgcaacagtccagagtagatgagcgttattactgtgccattaacaaatgcaaaacctaactttatcaggctttcatagcaacttgctttcctacagaaaatcctgttgcttacagaatttcctgccacaaacatttttgggaagccctcaggacacttacacaggcttgtgaatcaatcacatgctcagcaggtactgttcagtgggatacacagtgtagcgatgcatcactctgaaatattttagtttctttttttcccctaaatctgggtatgttcgtgggaatttgcagcacatgtgaacaacttctgtcattcttgcatgaggcaaagggaattgaaaaccacgattactttagaaaactagtttcacagattggtcactgtataaaagaaggatattggttttggtagcttgtgaccacacaccatttctgatctgaataaattcagaacttataatacagttcagaaattgaatgcagtttctcaatatgaggaaagtattttagaataaggcctatttttcaaaggatctgtggaaatcaatgctatgctctcatttaggagatggaaagagtgaggttaaattatcatttcgattaaatctacagtccagattactggtggatgaattgaatgtacttttttattcatataaaacatttgaaatcagaaatctggagtacttttaaatcccattatttattttgttttaatcgccaggtaattcctgagacaggagtgtcccgaagagcctttgcaattatgtaagaatctccgaggcagttcttatgttcctcaattcaaaagaaccacataactgcaatttaaataacaccccacacacacacaaaataaggtcgaagtttatctcaaaataatttcccctctctacactgggataaatatgtataggaataatagggggaaattcagtgcactgagcattaagctgtcaaaacaggaatgtttaaaatatcctgttagtggtttaaaaataatttgtactctaagtccagtgactatttgccagggagaaccaaagttgagaaatttctattaaaaacatgactcagagaaaaaaatgcagaggccggtaatgaaggaaatgattggatctcattcccaattggtcattcctaagatcacatgttctgagcatctttaaaaggaagttatctggactcaagagggtcacagcaccctcctgaaaac

SEQ ID NO: 63 - DNA, 651 bp, unidentified
  source 1..651: mol_type = unassigned DNA
gattaacccgccatgctacttatctactcgacattgattattgactaggggaattccagcaggaatcaggtagctggagaatcgcacagagccatgcgattcttggcaagccatgcgacaaaggcagaaatgcacatttcacccagagagaagggattgatgtcagcaggaagtcaccacccagagagcaaatggagttcccagatgcctgacatttgccttcttactggatcagagttcactagtggaagtgtcacagcccaaacactcccccaaagctcagcccttccttgccttgtaacaatcaagccgctcctggatgaactgctccgccctctgtctctttggcagggttggagcccactgtggcctgagcgacttctatggctcccttttctgtgattttcatggtttctgagctcttttcccccgctttatgattttctctttttgtctctctcttgctaaacctccttcgtatatatgccctctcaggtttcatttctgaatcatctactgtgaactattcccattgtttgccagaagccccctggttcttccttctagacaccaggcaaggggcaggaggtaagaggcaggagtccataaaacagccctgagagcctgctgggtcagtgcctgctgtcagaa

SEQ ID NO: 64 - DNA, 503 bp, unidentified
  source 1..503: mol_type = unassigned DNA
ctggcctctgacttacttggatgggaaatagccaaaaaaaaaaaaaatgctgaatatctcagatataggtaatgggtcttcaccagaacataaaaatagacccttattagccatatcagtaatgtgctgtgtgggggattttttttttctttcaattcctctggcaacaaaccacacactgggatctgacactgtagagtgctttctcttctctttttttgggggggggagggggtgtggttgcatatttaaactctcacgcatttatgtaatgaggactgcagtgtaggactttcctgcagaataccatttgatcctattaagaattgtccaaatgttggagcatttgattgaaaaatccttcttagccattttaaaggtaagttgtatgatttttctttaaataaaaaagattaagggatttttcccatcacccagatgacgggaggtgacactactgtataaatactcctaagaagttatataggaggacaggaaaaaag

SEQ ID NO: 65 - DNA, 1000 bp, unidentified
  source 1..1000: mol_type = unassigned DNA
caggtgtgagccactgcacccggccactcccaaatggttctgatgccttaagtggaactaccattgagtcaacacatattcatattatgctgactctatcccagatactgaaaaggacactaaggatgcaaaggtgattccaacagacatggtacctgtctagggattgttcagtctagacacaaatgacagccatggaatgctccaattaaaagaaaaatattttgagtaaaactatttaaaacactggtaatcacattatcaggattcattcacggccgggcgcggtggttcacgcctgtaatcccagcactttgggaggccgaggggagtggatcacgaggtcaggagatggagaccatcctggctaacacggtgaaaccccgtctctactaaaaatacaaaaaattagccgggcatggtggcgggcacctgttgtcccagctactcgggagtctgaggcaggagaatggcgtgaacctgggaggcggagcttgcagtgagccgagatcgcgccactgcacaccagcctggatggcagagcgagactccgtttcaaaaaaaaaaaagaaagaaaagattcattcacagtaaacaaacaaacaaacaaacaaaaaactcttcgttgatttcaatctggaaatgaaaaagaaaagaagaaaaggaaaggaaaaagaattctgactcattgatagatttgtttagaaatgtttttcttttttctgttagaaagctcagagttaaagagtggacaagagaaataacattttttcaactcttccattttctaaataaaacactgccactatacattggagtatgggatattatttccactttaaagataaggaaactgactcaaatacacggaaaacaggatttagtaaacgtgatttatttggaacgtgatctcagatccacagaggtgagaaaaacaggaaaggagaaagaaaaagtgctgattgagagtgtggtctcagctggagacgctcttaactct

SEQ ID NO: 66 - DNA, 1921 bp, unidentified
  source 1..1921: mol_type = unassigned DNA
ctgcaggagtattctatataagatagcaggcagttctgcacgtctgtttatatgccattcacaccatgaacacatccaatgcaattgtattcatcttctgtcattttttaaaattagagtttactacatgatttttggaggacggccatatcatagttattcatcccttgcatggtctgttctcgccacttctaaagttctgcgaagtttaatttttggcaggtttcaagattctctacatactttgtaggttagagtgaacgtgcacagtatattgtgtaataattgcaatacaagacggtgtgttgtgttgtggcacggaatgcatcaacttaaagatagaattgttatgcagaatagagacctttgaaatatttgggtattatcaacttttatttcatttttgaggaaacttaaaagtgagcccagtttttaaaaagcattcattgttctaacctacaattgtgcattgtatgtttgtgtgtgtgtgacggagggtgggatgaataaaaggggacattaaaaatattcagaccataaatatagtagattggaggttctgatttgatgagccagtttctcagccataaactgagagggggttggggatacacttcattgtccttcctggctaatgtaaatcccttatatttaaaaagatatttaaaagtattccaagcattttggcagaaaagcatagtatctaatgttatttttttcttattttatgtgcatgcctcttatccatttaaaaatagctttactgacctataatttacacactatataattctcccattgaaagtgcataattctgttgcttttagtatatttacagagttgtgcagcatcagcataatgtaatctagaacattgtcatcaactacccccaaatctctattcttcccttcccctattaattacccagccccaggcaagcactgatctacttttggtctctatggatttgtctatttgtggacactttaaatggaatcatacaatatgtgtcttttgcgactatcttctttcacttatcataactcaatacggctttagattatttgacctcgatgttctgcctctgaacataaaatattatccttgcattccttgatgagtttgaggattgagaataatttgcatgagacaaaaattagaaactagttagagcaagtaggcttttctccatcacataagctgatccatcttgccaatgttaaaacaccagattgtacaagcacaagctgggacgcaatgtgtgtccctctatccctacattgactttgcgggggtggggatggggtgcggggtgagtgagggaggactgcaagtgacaaacaggattcgtcaaaagagaggtgtattaaagtgccgatcaaatgtaatttaacagctaaactttccctccttggaaaacaggtgattgttgagtatttaacgtgaatcgatgtaaacctgtgtttggtgcttagacagggggcccccgggtagagtggaaccccttaagctaagcgaacaggagcctaacaaagcaaatttttccgtctgccctttccccctcttctcgttggcagggttgatcctcattactgtttgctcaaacgtttagaagtgaatttaggtccctccccccaacttatgattttatagccaataggtgatgaggtttatttgcatatttccagtcacataagcagccttggcgtgaaaacagtgtcagactcgattccccctcttcctcctcctcaagggaaagctgcccacttctagctgccctgccatcccctttaaagggcgacttgctcagcgccaaaccgcggctccagccctctccagcctccggctcagccggctcatcagtcggtccgcgccttgcagctcctccagagggacgcgccccgagatggagagcaaagcc

SEQ ID NO: 67 - DNA, 650 bp, unidentified
  source 1..650: mol_type = unassigned DNA
cctcgatgttctgcctctgaacataaaatattatccttgcattccttgatgagtttgaggattgagaataatttgcatgagacaaaaattagaaactagttagagcaagtaggcttttctccatcacataagctgatccatcttgccaatgttaaaacaccagattgtacaagcacaagctgggacgcaatgtgtgtccctctatccctacattgactttgcgggggtggggatggggtgcggggtgagtgagggaggactgcaagtgacaaacaggattcgtcaaaagagaggtgtattaaagtgccgatcaaatgtaatttaacagctaaactttccctccttggaaaacaggtgattgttgagtatttaacgtgaatcgatgtaaacctgtgtttggtgcttagacagggggcccccgggtagagtggaaccccttaagctaagcgaacaggagcctaacaaagcaaatttttccgtctgccctttccccctcttctcgttggcagggttgatcctcattactgtttgctcaaacgtttagaagtgaatttaggtccctccccccaacttatgattttatagccaataggtgatgaggtttatttgcatatttccagtcacataagcagccttggcgtgaaaacagt

SEQ ID NO: 68 - DNA, 247 bp, unidentified
  source 1..247: mol_type = unassigned DNA
ggtacccaagggtgcgcgcgtggctcctggcgcgccgaggccctccctcgaggccccgcgaggtgcacactgcgggcccagggctagcagccgcccggcacgtcgctaccctgaggggcggggcgggagctggcgctagaaatgcgccggggcctgcggggcagttgcgcaagttgtgatcgggccgctataagaggggcgggcaggcatggagccccgtaggaatcgcagcgccaacggttgcaag

SEQ ID NO: 69 - DNA, 1131 bp, unidentified
  source 1..1131: mol_type = unassigned DNA
cttcaacagtcctgcaagtcacaaggcttgttgaccacggtgaccctttctgagagccagcggaggaagagggcgggcatagggtcaggtcttaagagatcaggtcagtctgctcaaaccaggtgggctcaggaggagccggcgagggtcccgctgcagctcccgaaacgctgtttccaggcggcagtcagcagttgggcacgagaggatatcctagcaagcctccatcccagaaagttacacctcttactgcccgagtcccacccctcaacactgagcccacctcttcagtcctgtagtgaggggttctgggaaccccttgtaactgcccgccttcctcggaggtctcagaggccctaattgcgtcttccgatgggaacaaccatgagagcagggtgtggacggaagtcccccagccccagcggggttcactctttgcctcctcactctgcgagggtcccttactacagctaaaaggcaattcatgcagtccaaaggctcctttcccctcccgttctgcatgcccttctcactgtccccaaatgctcatgacatggccttgagagcagcgcagcgggcaggaagcccaggcgcctcggagaacctgaagggctgcaggtcccggtgtctgcggcagcttccgcatccctgcagggaaccagcggctcgctgagcctggggctgggctctgggggactccagagctggaggcaagtggcgctcacatccgtctcctcacctgccccacccgtggcctgggtttaaaatccacatacccgtctttccgcggccaaagtgatgctgccaggattggttatgaccccaactgccccgacccccagaagtgcaaaacgaacaggctggcaagtgaccaaaagagacccggggagcatctgggcttccaaggtcctcggtacggcccaaggcagcgaaggacgcgcggctccaggctgcgggagccaggacgaccgggggctcccagagcgcgaagtcgcgatcctcggcggtggagagctcgtgccaaaacgtcctcccctgcgccagtcaggccttcgcggggctggcaggcgggcgggggcggggccgccgcactttaagaggctgtgcaggcagacagacctccaggcccgc

SEQ ID NO: 70 - DNA, 778 bp, unidentified
  source 1..778: mol_type = unassigned DNA
gggggcgtgcatatcttagctggggagaaggcaactggggagtacctcttggcaagtgacaagctgaggcccagacaaggaatctgtggaatgacacgggtgaatgacatgggtgaggaatctctctctctgagcctctgtttccctgtgtgaggtgggatgccagtcacagcagggtgggctgagaggggtaagtggcaggtccaggttacccagtgtggtcacaccacccagtaggaccgacaggcagctcaggcccctagctgagcctggcactgcccttaatcggcttttgtccttccaggagggacaggcagggctggctacctcctctctccccccaacagctggggctggctgtgccagggccagactaggaggcgggagctgagatcactgggcctttggctccctgagtcctctctggggacagagagggcagcctggctgggtgagaggggacggtttgttcaggactggggctaggactcctgggtcctgaaggatgaaggggctgggggcctggactcctgggtctgagggaggaggggctatgggtgtggactcctgggttctaaggcagaggagcctgtaacaggactaccagttgagctgagtctgggagcaggtggggggcagagcagggagctgagccctctactctgtttacagcacgtggtcctcactgatctttctgggtgggaggtggcttgtgcggctacaccctgggcaggccagccccgcccccgggtttattgccccaggctgctactggcac

SEQ ID NO: 71 - DNA, 1822 bp, unidentified
  source 1..1822: mol_type = unassigned DNA
taatcctcaccactaccctataaaacaagaatcattatcgtcattacacagagaagagaaacgggctcacagaagtcaagtgatctgtctaagctcaagcaagctgatatggggtggagttgggaccacaacccacatctgcctgctttctgtgccctctgtgcttgatctcatgtcatgggccatccattttgccaaagctttcgaaacatgccttctcccctccttttaggccttttctggaagtcaggctggcttgcagctctcaccccaagctcttttccccagctggggctgaaggctggggaggagcttcatgggggctggaaataaatgaagggagggctgggcgggacagggcagtatttccaggaggctgtgagggggaggtaaggagtttgttattgttttgggtttggggatttttttttctttgctcctggagagcaggttaggtagctcttggcagagttttgctagggatacgaagtgggtgaggctgaggcaagcagaatcctcctgcctcagcctcccacgtagctaggactagagcttctgcagtgctaagtgggaaaggggctttctgcctctgtggggcctggtctggaaagtcccccaaattgttaaaagcaaagagtgattgtccccagctggcaaggggacttttgttttatgtccaccccttgtgccccttgctctgcgaagccctacctgtgccgggagcttcgaataggaagaggacctcagggccaggcatccagggactgaggggctacagcagaaacagacccagaggcacccttggctaggaagagcaggatgaaggtgtaataattttgggagtggtctaagaagactttccttttaaagagtggaaagcaatgaaactgaggcttcaaggctgcaaaaatttcgtcctggatcagagaagggcactacaggtggagggaactatctgtgcaaagggttgggcccttcaagagttggagcacagaggtttaggagaaaaagtggtgacagatgagcccagggctatctgcttagacctgtgatctaatatgggtgccaacagccacatggcaatgtggctatttaaatttaaattagttaaaattcaataaagtaaaaaatacagctcctgtgccaggtgcagtggctcacgcctgtaatccccgcactttgggaggccaagacagggagatcacctgaggtcaggagttcaagaccagcctagccaacatggtgaaacccagtctctactaaaaatccaaacaaaacgaaactaaactaaaaattagctgggcgtggtggcaggtgcctgtaatcccagctacgggggaggctgaggcaggataattgcttgaacccaggaggcggaggttgtagtgagcaagatcgccccattgcactccagcctgggtgacaagagcgaaactcaaaaaaaaaaaaaattcagctcctgaatcacactagttatgtttcaaatactcaatagccacatggggctagtagcaatggtattggagagtatgtacaaggcatgcctctatcatcacagaatgttcttttggccactgtgaagcctcaggaaggggctcggattgctcaaggacccatgggagagaggaggctttgactgggctgcctgcctgtgaggtctctggactagaggtgagggaacggtccccagaagtgtgaggtggtgtgactcacctcctgccagcgtaaagtggactctgtccccatggacaggaacaaatcgggcaaggcttcttcagccctgagttgggttatctgctcagtgtgaccagagccaggggat

SEQ ID NO: 72 - DNA, 1692 bp, unidentified
  source 1..1692: mol_type = unassigned DNA
acaggcacccaccaccacgctaatttttgtatttttagtagagacagggttttgccatgatggccaggctggtctcgaactgctgacctcaagtgatccacctgcctcagcctcccaaagtgctgggattacaggcatgagtcaccgtgcccggctgagttttcaaatcttattcatttccaggtgtcctttcagccccatatctcccactgagctgtgcggcaggatctcggctcactgcagcctggacctcccaggctcaagcgattctcctccctcagcctcccaagtagctgggattacaggcatgtaccaccacgcccatatattttttgtattttttgtagagatgggattttgccatgttgcccaggctggtgtcaaactcctgggctaaagcagtccttctgcctcggcctcccgaagtgctgggtttatgggcatgagccatggcatcccgcctgatcgtaggattttaaactatttcatacaaccactttcaaaccacacacacacatccgtggcggctccagatcacacttgtaaaacaatgtaaggaggggcgtcaggaatatagaaagagatgccgaggatagagaagaaggcagagacagagggagaaaacctaagagagaacaaaggaggagaatccgataggacaaacagcctgaggcccttccgagctggcatctgcccggagcagctgacctaggggctctctgccaccggctctttagcaccccagaatcgttctgatgggcatgaggtggacccaacactcccccttatcctcttccccacctggtggtgagctacgtagaaacctactcatggggccctgctggtccatgcagggtccaggcaggatccaggggctaccctggtgctcacatctccccttcccttctggagtcgggagccaggctttggcctggatgctcccagcggttgggggtcagggtctgggtgggatgtaatagggggccctaaggtccagcatttcccaacagaaagaaagaagtacacgcttgctttggaagaagcagcggagggctcagttcccaggacagctgaagacacatccctgtggggcccaaagtgtctttctccccatccagagaccctgcccagaggagcaggccctggagctgcaacagaaaccggaggctggcccaagtccctctgggctgggctgggctgggctgggcagagctgggctgggctgggctgggctggggcagaggcaaaatcctgccagggtggttctctggggagaggggaattgaaaaggaaaagcatcaccaaggagggggagagggagggaggccgtgtgcgcacacgcacacacacacacacacacacacacacacacacacagcctccctgactgagcctgagcctccctgggaagaggcgcattcttctctctgctctctgagctttccgttgcttccctgcccatctgtgctctcagcagcaccagcaaagttggcctcaaacttgaacaaagctgcaggctccagcgtctttctgggagctccgcgggggcagcaggcaggccccagcctctgaactcagctggcttgagaagctcagcccaacccttccagcacccagccggccctccgtctcctgagggacgcccctgcccccgacctggaggccctcagtctgggctggggaatggcgcgc

SEQ ID NO: 73 - DNA, 67 bp, unidentified
  source 1..67: mol_type = unassigned DNA
ctagagtcgactccataaagtaggaaacactacacgattccataaagtaggaaacactacaaccggt

SEQ ID NO: 74 - DNA, 57 bp, unidentified
  source 1..57: mol_type = unassigned DNA
tgtagtgtttcctactttatggaatcgtgtagtgtttcctactttatggagtcgact

SEQ ID NO: 75 - DNA, 51 bp, unidentified
  source 1..51: mol_type = unassigned DNA
tccataaagtaggaaacactacatcactccataaagtaggaaacactacac

SEQ ID NO: 76 - DNA, 61 bp, unidentified
  source 1..61: mol_type = unassigned DNA
tcgagtgtagtgtttcctactttatggagtgatgtagtgtttcctactttatggaaccggt

SEQ ID NO: 77 - DNA, 75 bp, unidentified
  source 1..75: mol_type = unassigned DNA
gatccacatgtcgtacgaccacacacttccttacattccacgataccacacacttccttacattccagtttaaac

SEQ ID NO: 78 - DNA, 63 bp, unidentified
  source 1..63: mol_type = unassigned DNA
tggaatgtaaggaagtgtgtggtatcgtggaatgtaaggaagtgtgtggtcgtacgacatgtg

SEQ ID NO: 79 - DNA, 63 bp, unidentified
  source 1..63: mol_type = unassigned DNA
accacacacttccttacattccacgataccacacacttccttacattccaaccggtacatgta

SEQ ID NO: 80 - DNA, 75 bp, unidentified
  source 1..75: mol_type = unassigned DNA
gatctacatgtaccggttggaatgtaaggaagtgtgtggtatcgtggaatgtaaggaagtgtgtggtgtttaaac

SEQ ID NO: 81 - DNA, 90 bp, unidentified
  source 1..90: mol_type = unassigned DNA
aggcctccacacacttccttacattgcatctaggccacacacttccttacattgcatctaggccacacacttccttacattgcatctaga

SEQ ID NO: 82 - DNA, 176 bp, unidentified
  source 1..176: mol_type = unassigned DNA
tctagaccacacacttccttacattccaggatccccacacacttccttacattccacgtacgccacacacttccttacattccaacgcgtccacacacttccttacattccacacgtgccacacacttccttacattccagcatgcccacacacttccttacattccagcggccgc

SEQ ID NO: 83 - DNA, 29 bp, unidentified
  source 1..29: mol_type = unassigned DNA
tcgacacaaacaccattgtcacactccac

SEQ ID NO: 84 - DNA, 29 bp, unidentified
  source 1..29: mol_type = unassigned DNA
aattgtggagtgtgacaatggtgtttgtg

SEQ ID NO: 85 - DNA, 45 bp, unidentified
  source 1..45: mol_type = unassigned DNA
taattctagatcgcgaacaaacaccattgtcacactccagtacta

SEQ ID NO: 86 - DNA, 122 bp, unidentified
  source 1..122: mol_type = unassigned DNA
ggccggccaatacatacttctttacattccacgataatacatacttctttacattccaaccggtaatacatacttctttacattccatcacaatacatacttctttacattccaggccggcc

SEQ ID NO: 87 - DNA, 23 bp, unidentified
  source 1..23: mol_type = unassigned DNA
actcaccgacagcgttgaatgtt

SEQ ID NO: 88 - DNA, 70 bp, unidentified
  source 1..70: mol_type = unassigned DNA
gatccctcgaggctgaggtcacagtgtgccttagagagctgagtcacagtgtgccttctcgaggtcgacg

SEQ ID NO: 89 - DNA, 71 bp, unidentified
  source 1..71: mol_type = unassigned DNA
gatccgtcgacctcgagaaaggcacactgtgacctcagctctcaaaggcacactgtgacctcagcctcgag

SEQ ID NO: 90 - DNA, 80 bp, unidentified
  source 1..80: mol_type = unassigned DNA
acaaagttctgtagtgcactgagagcttgacaaagttctgtagtgcactgagagcttgacaaagttctgtagtgcactga

SEQ ID NO: 91 - protein, 5 aa, synthetic construct
SGGGS

(19) United States
(12) Patent Application Publication
(10) Pub. No.: US 2026/0109963 A1
(43) Pub. Date: Apr. 23, 2026

(54) COMPOSITIONS, SYSTEMS, AND METHODS FOR REDUCING ADIPOSE TISSUE

(71) Applicant: Oisin Biotechnologies, Inc., Seattle, WA (US)

(72) Inventors: Matthew Rein SCHOLZ, Seattle, WA (US); Gary Charles HUDSON, Seattle, WA (US)

(21) Appl. No.: 19/281,400

(22) Filed: Jul. 25, 2025

Related U.S. Application Data
(63) Continuation of application No. PCT/US24/13231, filed on Jan. 26, 2024.
(60) Provisional application No. 63/441,611, filed on Jan. 27, 2023.

(51) Int. Cl. C12N 9/64; A61K 48/00; C12N 15/85; C12N 15/88

(52) U.S. Cl. CPC C12N 9/6472; C12N 15/85; C12N 15/88; C12Y 304/22062; A61K 48/00; C12N 2830/008

(57) ABSTRACT

Provided polynucleotide constructs for the adipocyte-specific production of a cytotoxic protein. Also provided are formulations and systems, including lipid-based delivery vectors for the delivery of polynucleotide constructs as well as methods for making and using such nucleic acid-based polynucleotide constructs, formulations, and systems for reducing growth and/or survival of adipocytes. Compositions, systems, and methods disclosed herein can be used for reducing adipocytes or adipose volume in visceral, subcutaneous, and abdominal fat.

### CROSS REFERENCE

This application is a continuation of International Application No. PCT/US24/13231, filed Jan. 26, 2024, which claims the benefit of U.S. Provisional Patent Application No. 63/441,611, filed Jan. 27, 2023, each of which is incorporated herein by reference in its entirety.

### SEQUENCE LISTING STATEMENT

The instant application contains a Sequence Listing which has been submitted electronically in XML format and is hereby incorporated by reference in its entirety. Said XML copy, created Jul. 25, 2025, is named 54636-711_601_SL.xml and is 114,802 bytes in size.

### BACKGROUND

Since 1975, worldwide obesity has nearly tripled; in 2016, more than 1.9 billion adults were overweight, of which over 650 million were obese. Accumulation of excess fat is associated with increased relative risk of a large number of diseases and health conditions, as well as psychological and emotional distress. Overweight or obese individuals have increased risk of cardiovascular diseases, stroke, diabetes, musculoskeletal disorders, and some cancers (including endometrial, breast, ovarian, prostate, liver, gallbladder, kidney, and colon).

Conventional interventions such as diet and exercise are ineffective in many cases due to poor compliance and other factors, and lipidemia diseases can be associated with genetic or hormonal factors that are challenging to address. Alternate interventions involving surgical procedures can be invasive and risky, thus there is a need for therapeutic strategies to reduce adipose tissue.

### SUMMARY

Disclosed herein, in some aspects, is a polynucleotide construct for selective killing of human adipocytes, comprising: (a) a transcriptional promoter that is preferentially or specifically active in a human adipocyte; and (b) a transgene encoding a cytotoxic protein, wherein expression of the cytotoxic protein is regulated by the transcriptional promoter.

In some embodiments, the polynucleotide construct comprises DNA. In some embodiments, the polynucleotide construct comprises double stranded DNA. In some embodiments, the polynucleotide construct is a plasmid. In some embodiments, the polynucleotide construct is a minicircle. In some embodiments, the transcriptional promoter comprises an ADIPOQ promoter or a functional fragment thereof. In some embodiments, the transcriptional promoter comprises a FABP4, PLIN1, PPARγ, PPARγ1, PPARγ2, CD36, LPL, LEP, CIDEC, TUSC5, CIDEA, or LIPE promoter or a functional fragment thereof. In some embodiments, the transcriptional promoter comprises a nucleic acid sequence with at least about 80%, at least about 85%, at least about 90%, at least about 95%, at least about 97%, at least about 98%, or with 100% sequence identity to any one of SEQ ID NOs: 1 and 56-72. In some embodiments, the promoter is at least 50% more active in the human adipocyte than a control cell that is not the human adipocyte. In some embodiments, the control cell is a myocyte, hepatocyte, osteocyte, erythrocyte, neuron, leukocyte, lymphocyte, or fibroblast. In some embodiments, the cytotoxic protein induces non-inflammatory cell death upon expression of the cytotoxic protein in the human adipocyte. In some embodiments, the cytotoxic protein induces apoptosis upon expression of the cytotoxic protein in the human adipocyte. In some embodiments, the cytotoxic protein comprises a caspase or a catalytic domain thereof. In some embodiments, the caspase comprises an inducible caspase or catalytic domain thereof. In some embodiments, the caspase comprises a rapamycin-inducible caspase. In some embodiments, the rapamycin inducible caspase comprises an FKBP-rapamycin binding (FRB) domain. In some embodiments, the rapamycin inducible caspase comprises an FK506-binding protein (FKBP) domain. In some embodiments, the FKBP domain is an FKBP12 domain. In some embodiments, the rapamycin inducible caspase comprises, from N- to C-terminus, the FRB domain, the FKBP12 domain, and the caspase or functional fragment thereof. In some embodiments, the caspase is a non-inducible caspase. In some embodiments, the caspase is a self-activating caspase. In some embodiments, the caspase comprises a caspase 9 or catalytic domain thereof. In some embodiments, the caspase comprises a caspase 1 or catalytic domain thereof. In some embodiments, the caspase comprises a caspase 3 or catalytic domain thereof. In some embodiments, the cytotoxic protein comprises a caspase 8, BAX, DFF40, HSV-TK, cytosine deaminase, or catalytic domain thereof. In some embodiments, the cytotoxic protein comprises an amino acid sequence with at least 80% sequence identity or sequence similarity to any one of SEQ ID NOs: 3-13 and 52-55. In some embodiments, the adipocyte is a white adipocyte. In some embodiments, the adipocyte is a not a brown adipocyte. In some embodiments, the polynucleotide construct further comprises a safety element that reduces expression of the cytotoxic protein in a control cell that is not the human adipocyte. In some embodiments, the control cell is a myocyte, hepatocyte, osteocyte, erythrocyte, neuron, leukocyte, lymphocyte, or fibroblast. In some embodiments, expression of the safety element is driven by a regulatory element that is active in the control cell but is less active or substantially inactive in the human adipocyte. In some embodiments, the safety element comprises a regulatory RNA that targets a transcript encoding the cytotoxic protein for degradation. In some embodiments, the safety element comprises a target site of a regulatory RNA, e.g., an endogenous or engineered regulatory RNA. In some embodiments, the regulatory RNA is a siRNA or miRNA. In some embodiments, the safety element comprises a transcriptional repressor that reduces expression mediated by the transcriptional promoter.

Disclosed herein, in some aspects, is a lipid-based delivery vector (LDV) comprising the polynucleotide construct of any one of the preceding embodiments.

In some embodiments, the LDV comprises a fusion-associated small transmembrane (FAST) protein. In some embodiments, the FAST protein comprises an ectodomain of a first reovirus FAST protein and an endodomain of a second reovirus FAST protein. In some embodiments, the FAST protein comprises p10, p13, p14, p15, p16, p22, or a functional domain thereof. In some embodiments, the FAST protein comprises a fusion of a first domain from a p14 FAST protein or a p10 FAST protein and a second domain from a p14 FAST protein or a p15 FAST protein. In some embodiments, the FAST protein comprises an ectodomain of p14 and an endodomain of p15. In some embodiments, the LDV comprises an ionizable lipid. In some embodiments, a molar ratio of the ionizable lipid to the polynucleotide construct is between about 2:1 and 25:1. In some embodiments, the molar ratio is about 5:1, about 7.5:1, about 10:1, or about 15:1. In some embodiments, the ionizable lipid comprises Dlin-KC2-DMA (KC2), DODMA, DODAP, DOBAQ, DOTMA, 18:1 EPC, DOTAP, DDAB, 18:0 EPC, 18:0 DAP, or 18:0 TAP. In some embodiments, the LDV is configured to deliver the polynucleotide construct to the human adipocyte upon contacting the human adipocyte with the LDV. In some embodiments, the LDV is configured to deliver the polynucleotide construct to the human adipocytes upon administration of the LDV to a subject. In some embodiments, the LDV is formulated for non-targeted delivery to the human adipocytes and to non-adipocyte cells.

Disclosed herein, in some aspects, is a cell comprising the polynucleotide construct of any one of the preceding embodiments.

Disclosed herein, in some aspects, is a method of reducing viability of a population of adipocytes, the method comprising contacting the population of adipocytes with the LDV of any one of the preceding embodiments under conditions that facilitate uptake of the polynucleotide construct by the adipocytes.

In some embodiments, the adipocytes comprise white adipocytes. In some embodiments, the adipocytes are human adipocytes. In some embodiments, during the contacting, the LDV containing the polynucleotide construct is present at a concentration of at least 1 nM. In some embodiments, at least about 1% of white adipocytes in the population are killed. In some embodiments, at most about 95% of white adipocytes in the population are killed. In some embodiments, between about 5% and 80% of white adipocytes are killed.

Disclosed herein, in some aspects, is a method of reducing an adipose tissue volume, the method comprising administering to a subject an effective amount of the LDV of any one of the preceding embodiments.

In some embodiments, the LDV is administered systemically. In some embodiments, the LDV is administered locally. In some embodiments, the LDV is administered via injection into adipose tissue. In some embodiments, the LDV is administered into visceral fat. In some embodiments, the LDV is administered into subcutaneous fat. In some embodiments, the LDV is administered into abdominal fat. In some embodiments, the adipose tissue volume is reduced by at least about 5%. In some embodiments, the adipose tissue volume is reduced by at most about 95%. In some embodiments, the adipose tissue volume is reduced by about 5% to about 80%. In some embodiments, the adipose tissue is white adipose tissue. In some embodiments, the reduction in adipose tissue volume is as determined by DEXA scans to quantify the adipose tissue before and after administering the LDV containing the polynucleotide construct. In some embodiments, the cytotoxic protein is an inducible caspase and the method further comprises administering an inducer of the caspase to the subject. In some embodiments, the cytotoxic protein is a rapamycin-inducible caspase and the method further comprises administering rapamycin or a structural analog thereof to the subject. In some embodiments, the LDV is administered to the subject two or more times. In some embodiments, the rapamycin or the structural analog thereof is administered to the subject two or more times. In some embodiments, the method treats lipedema in the subject. In some embodiments, the method treats a metabolic disorder in the subject. In some embodiments, the method treats Dercum's disease in the subject.

Disclosed herein, in some aspects, is a system for selective killing of human adipocytes, comprising the polynucleotide construct of any one of the preceding embodiments, rapamycin or a structural analog thereof, and optionally the LDV of any one the preceding embodiments.

### BRIEF DESCRIPTION OF THE DRAWINGS

FIG. 1 provides images of H&E sections of human tissue explants, showing reduced fat content after treatment delivery of a polynucleotide construct, expression of iCasp9, and treatment with a chemical inducer of dimerization.

FIG. 2 quantifies the change in percent of adipose tissue area in H&E sections of human tissue explants after treatment delivery of a polynucleotide construct, expression of iCasp9, and treatment with a chemical inducer of dimerization.

FIG. 3 shows in vivo expression of a reporter gene in fat tissue of mice dosed with an LDV formulation comprising a polynucleotide construct with reporter gene expression driven by the adiponectin promoter.

FIG. 4 shows in vivo expression of a reporter gene in fat tissue of mice dosed with an LDV formulation comprising a polynucleotide construct.

FIG. 5 illustrates collection of skin and fat pad samples near the site of injection of a polynucleotide construct.

FIG. 6 illustrates expression of a reporter gene in fat tissue of mice dosed with an LDV formulation comprising a polynucleotide construct.

### DETAILED DESCRIPTION

The present disclosure provides polynucleotide constructs, systems, and methods for the selectively reducing growth and/or survival of an adipocyte, which can be referred to as a “target cell”.

Provided compositions, systems, and methods can be used for reducing the growth and/or survival of adipocytes, and can reduce adipose tissue volume. The adipocytes can be associated with excess fat, lipedema, lipidemia diseases, metabolic disorders, and other conditions. Polynucleotide constructs are provided for target cell-specific expression of therapeutic proteins, for example, cytotoxic proteins. The polynucleotide constructs can utilize intracellular features, including transcription regulatory features, that are present within a target cell but absent from or substantially reduced in a control or non-target cell. Such polynucleotide constructs are used in systems that include a vector for delivery of the polynucleotide construct to a target cell, including lipid-based delivery vectors (LDVs).

### I. POLYNUCLEOTIDE CONSTRUCT

Disclosed herein are polynucleotide constructs and systems and methods that comprise the polynucleotide constructs. A polynucleotide construct can comprise an expression regulatory region to drive or control expression of one or more transgenes, such as a transgene that encodes a cytotoxic protein for inducing death (e.g., apoptosis) of adipocytes, an extracellular matrix remodeling factor, and/or a safety element.

A polynucleotide construct disclosed herein can be or can comprise DNA. A polynucleotide construct can be or can comprise double stranded DNA. For example, a polynucleotide construct disclosed herein can be or comprise a plasmid, such as a nanoplasmid. In some embodiments, a polynucleotide construct disclosed herein is or comprises a minicircle, a midge, a MIP, or a doggy bone. A polynucleotide construct disclosed herein can be or can comprise a circular polynucleotide. A polynucleotide construct disclosed herein can be or can comprise a linear polynucleotide. A polynucleotide construct disclosed herein can comprise an RNA, e.g., a circular RNA.

In some embodiments, a polynucleotide construct or polynucleotide disclosed herein is not single stranded DNA. In some embodiments, a polynucleotide construct or polynucleotide disclosed herein lacks a component of a viral genome or lacks a viral packaging element, for example, lacks a 5′ and/or 3′ inverted terminal repeat (ITR).

In some embodiments, a polynucleotide construct or polynucleotide disclosed herein is non-integrating, e.g., does not integrate into the genome of a host cell.

### A. Promoter and Expression Regulatory Region

Polynucleotide constructs, systems, and methods disclosed herein can exploit the cell-specific or cell type-specific transcription regulatory machinery that is intrinsic to a target cell, such as an adipocyte (e.g., a white adipocyte). A polynucleotide construct can be used for targeted production of a cytotoxic protein in an adipocyte (e.g., a white adipocyte).

A polynucleotide construct disclosed herein can comprise an expression regulatory region. An expression regulatory region can comprise, for example, a promoter (e.g., an adipocyte-specific promoter), an enhancer, an intron, an exon or, a functional fragment thereof, or a combination thereof. A polynucleotide construct can comprise multiple expression regulatory regions, for example two expression regulatory regions, or more.

The transcriptional promoter can be a promoter as found in a naturally-occurring genome. In some embodiments, a promoter is not found in a naturally-occurring genome. In some embodiments, the promoter is an engineered promoter. The promoter can be a minimal promoter or a functional fragment of a larger promoter effective to drive expression of a downstream transgene in a target cell.

A polynucleotide construct disclosed herein can comprise a transcriptional promoter that is activated in a target cell, such as an adipocyte (e.g., a white adipocyte). The transcriptional promoter can be specifically, selectively, or preferentially activated in the target cell (e.g., adipocyte) as compared to a control cell (e.g., a non-adipocyte such as a myocyte, hepatocyte, osteocyte, erythrocyte, neuron, leukocyte, lymphocyte, monocyte, or fibroblast). In some embodiments, a control cell can comprise a brown adipocyte. The promoter can be specifically, selectively, or preferentially derepressed in the target cell as compared to the control cell. Transcriptional promoters that can be suitably employed in the polynucleotide constructs, systems, and methods of the present disclosure include transcriptional promoters that are capable of driving the expression of a transgene in a target cell (i.e., an adipocyte), but incapable of, or exhibit a substantially reduced capability of, driving expression of that transgene in a control cell.

In some embodiments, the transcriptional promoter drives a level of expression in an adipocyte (e.g., a white adipocyte) that is at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 250 fold, at least 500 fold, at least 1000 fold, at least 5000 fold, or at least 10000-fold higher than a level of expression in a control cell disclosed herein (e.g., a hepatocyte, osteocyte, erythrocyte, neuron, leukocyte, lymphocyte, monocyte, fibroblast, or brown adipocyte). The adipocyte and the control cell can each be primary cells. In some embodiments, the transcriptional promoter drives a level of expression in an adipocyte (e.g., a white adipocyte) that is detectable in the adipocyte (e.g., white adipocyte), and expression is undetectable or below a limit of detection in the control cell. Expression can be determined by, for example, a detectable reporter gene (e.g., fluorescent or luminescent protein or substrate), ELISA, western blot, etc.

A transcriptional promoter used in a composition, system, or method disclosed herein can be responsive to one or more factors that are specifically or preferentially produced within a target cell, such as an adipocyte.

A transcriptional promoter itself can be the primary mechanism by which adipocytes are preferentially targeted in a system or method disclosed herein. In some embodiments, a polynucleotide construct disclosed herein that utilizes an adipocyte-specific (e.g., white adipocyte specific) promoter overcomes safety and/or efficacy limitations that are associated with technologies that rely on targeted delivery of a therapeutic compound. For example, an adipocyte-specific promoter can reduce off-target effects, e.g., limit off target effects resulting from transgene expression in non-adipocytes, and use of a delivery vector, such as an LDV disclosed herein, can improve delivery of the polynucleotide construct to the adipocytes compared to an alternate delivery vector.

In some embodiments, a transcriptional promoter disclosed herein reduces or eliminates the need for a targeted delivery vector, e.g., that targets adipocytes using proteins or antibodies directed to adipocyte-specific surface molecules. For example, a system can use a selective promoter (e.g., an adipocyte selective promoter) for expression only or preferentially in desired cell type(s), without selective uptake of the polynucleotide construct by adipocytes over other control cells disclosed herein.

A transcriptional promoter used in compositions, systems, and methods disclosed herein can be a promoter that is known to be active in adipocytes (e.g., white adipocytes).

A transcriptional promoter used in compositions, systems, and methods disclosed herein can be an adiponectin promoter, or a functional fragment thereof, such as a core or minimal adiponectin promoter. In humans, adiponectin is encoded by the ADIPOQ gene. Adiponectin can function as an adipokine which is secreted by adipocytes. Adiponectin can function as homeostatic factor for regulating glucose levels, lipid metabolism, and insulin sensitivity. In humans, adiponectin is located on chromosome 3 (3q27). Adipocytes can show a high level of expression of adiponectin which can contribute to adipocyte differentiation. In adipocytes, CCAAT/enhancer-binding protein α (C/EBFα), peroxisome proliferator-activated receptor γ (PPARγ), sterol regulatory element-binding protein (SREBP)-1c, forkhead box 1, and specificity protein 1 can upregulate the expression of adiponectin and can be involved in promoting adipogenesis and increasing lipid content and insulin directed glucose transport, while reactive oxygen species, TNFα, and IL-6 can downregulate the expression of adiponectin.

A transcriptional promoter used in compositions, systems, and methods disclosed herein can be a mammalian adiponectin promoter, or a functional fragment thereof, such as a core or minimal mammalian adiponectin promoter. A transcriptional promoter used in compositions, systems, and methods disclosed herein can be a human adiponectin promoter, or a functional fragment thereof, such as a core or minimal human adiponectin promoter. In some embodiments, an adiponectin promoter comprises about 100, about 200, about 500, about 1000, about 1500, about 1600, about 1700, about 1800, about 1900, about 2000, or about 2500 base pairs upstream of the human adiponectin transcriptional start site. In some embodiments, an adiponectin promoter comprises at least about 100, at least about 200, at least about 500, at least about 1000, at least about 1500, at least about 1600, at least about 1700, at least about 1800, at least about 1900, at least about 2000, or at least about 2500 base pairs upstream of the human adiponectin transcriptional start site. In some embodiments, a polynucleotide construct or expression regulatory region disclosed herein comprises or utilizes an adiponectin enhancer element. An illustrative adiponectin promoter or fragment thereof is provided in SEQ ID NO: 1.

A transcriptional promoter used in compositions, systems, and methods disclosed herein can be a Fatty Acid Binding Protein 4 (FABP4) promoter, or a functional fragment thereof, such as a core or minimal FABP4 promoter. FABP4, also known as adipocyte P2 (aP2), is a fatty acid-binding protein specific to adipocytes, playing roles in fatty acid uptake, transport, and metabolism. FABP4 is highly expressed in adipocytes and its expression is highly induced during adipocyte differentiation. Its expression can be negligible in preadipocytes and increase substantially during adipocyte differentiation. FABP4's promoter/enhancer region, particularly a 540 bp enhancer, can be crucial for its adipose-specific expression gene. FABP4 is involved in the regulation of glucose and lipid metabolism in relation to inflammatory and metabolic processes. The expression of FABP4 is transcriptionally controlled by peroxisome proliferator-activated receptor (PPAR) γ agonists, fatty acids (FAs), dexamethasone, and insulin.

A transcriptional promoter used in compositions, systems, and methods disclosed herein can be a mammalian FABP4 promoter, or a functional fragment thereof, such as a core or minimal mammalian FABP4 promoter. A transcriptional promoter used in compositions, systems, and methods disclosed herein can be a human FABP4 promoter, or a functional fragment thereof, such as a core or minimal human FABP4 promoter. In some embodiments, a polynucleotide construct or expression regulatory region disclosed herein comprises a FABP4 enhancer. In some embodiments, a polynucleotide construct or expression regulatory region disclosed herein comprises an FABP4 (aP2) minimal promoter, e.g., comprising the approximately 540 bp adipose-specific aP2 enhancer linked upstream of the basal aP2 promoter at bp-63 (e.g., as provided in SEQ ID NO: 63). This mini promoter can exhibit increased specificity for adipocytes over, e.g., heart and skeletal muscle cells.

In some embodiments, a FABP4 promoter comprises about 100, about 200, about 500, about 1000, about 1500, about 1600, about 1700, about 1800, about 1900, about 2000, or about 2500 base pairs upstream of the human FABP4 transcriptional start site. In some embodiments, a FABP4 promoter comprises about 5400 or about 5403 base pairs upstream of the human FABP4 transcriptional start site. In some embodiments, a FABP4 promoter comprises at least about 100, at least about 200, at least about 500, at least about 1000, at least about 1500, at least about 1600, at least about 1700, at least about 1800, at least about 1900, at least about 2000, or at least about 2500 base pairs upstream of the human FABP4 transcriptional start site. In some embodiments, a polynucleotide construct or expression regulatory region disclosed herein comprises or utilizes a FABP4 enhancer element. Illustrative FABP4 promoters or fragments thereof are provide in SEQ ID NOs: 61-63. In some embodiments, a FABP4 promoter is as deposited in GenBank AJ627200.1, as described in US2020/0102361A1, or as described by Rival et al. “Human adipocyte fatty acid-binding protein (aP2) gene promoter-driven reporter assay discriminates nonlipogenic peroxisome proliferator-activated receptor γ ligands.” Journal of Pharmacology and Experimental Therapeutics 311.2 (2004): 467-475, each of which is incorporated herein by reference for such disclosure.

A transcriptional promoter used in compositions, systems, and methods disclosed herein can be a Perilipin 1 (PLIN1) promoter, or a functional fragment thereof, such as a core or minimal PLIN1 promoter. PLIN1 is a lipid droplet-associated protein that can have a role in protecting lipid droplets from hormone-sensitive lipase. PLIN1 has been described as primarily expressed in adipose tissue. For example, PLIN1 has been described as having strong specificity of expression in adipose tissues, including in both subcutaneous and omental fat (and e.g., 2-fold higher expression in subcutaneous vs visceral fat).

A transcriptional promoter used in compositions, systems, and methods disclosed herein can be a mammalian PLIN1 promoter, or a functional fragment thereof, such as a core or minimal mammalian PLIN1 promoter. A transcriptional promoter used in compositions, systems, and methods disclosed herein can be a human PLIN1 promoter, or a functional fragment thereof, such as a core or minimal human PLIN1 promoter. In some embodiments, a PLIN1 promoter comprises about 500, about 1000, about 1500, about 1600, about 1700, about 1731, about 1800, about 1900, or about 2000 base pairs upstream of the human PLIN1 transcriptional start site. In some embodiments, a PLIN1 promoter comprises about 100, about 200, about 500, about 1000, about 1500, about 1600, about 1700, about 1800, about 1900, about 2000, or about 2500 base pairs upstream of the human PLIN1 transcriptional start site. In some embodiments, a PLIN1 promoter comprises at least about 100, at least about 200, at least about 500, at least about 1000, at least about 1500, at least about 1600, at least about 1700, at least about 1800, at least about 1900, at least about 2000, or at least about 2500 base pairs upstream of the human PLIN1 transcriptional start site. In some embodiments, a polynucleotide construct or expression regulatory region disclosed herein comprises or utilizes a PLIN1 enhancer element. Illustrative PLIN1 promoters or fragments thereof are provide in SEQ ID NOs: 56 and 57. In some embodiments, a PLIN1 promoter is as described in Bialesov et al. “Epigenetic regulation of PLIN1 in obese women and its relation to lipolysis.” Scientific reports 7.1 (2017): 10152, which is incorporated herein by reference for such disclosure.

A transcriptional promoter used in compositions, systems, and methods disclosed herein can be a Peroxisome proliferator-activated receptor gamma (PPARγ) promoter, or a functional fragment thereof, such as a core or minimal PPARγ1 or PPARγ2 promoter. PPARγ is a nuclear receptor and a prominent regulator of adipocyte differentiation. It activates upon binding with a ligand and modulates the transcription of target genes involved in lipid storage and metabolism by binding to specific PPAR response elements (PPRE) in DNA. Mutations in PPARγ are linked to conditions like partial lipodystrophy, with its dysfunction resulting in the absence of discernible adipose tissue. Alternatively spliced transcript variants that encode different isoforms have been described (i.e., PPARγ1 and PPARγ2). Forced expression of murine PPARγ2 in fibroblasts can be sufficient to drive adipocyte differentiation, suggesting a role as an adipocyte-specific transcription factor. PPARγ mRNA has been described as being at least 5 times or at least 20 times more abundant in adipose tissue than most other tissues.

A transcriptional promoter used in compositions, systems, and methods disclosed herein can be a mammalian PPARγ promoter, or a functional fragment thereof, such as a core or minimal mammalian PPARγ promoter. A transcriptional promoter used in compositions, systems, and methods disclosed herein can be a human PPARγ promoter, or a functional fragment thereof, such as a core or minimal human PPARγ promoter. A transcriptional promoter used in compositions, systems, and methods disclosed herein can be a mammalian PPARγ1 promoter, or a functional fragment thereof, such as a core or minimal mammalian PPARγ1 promoter. A transcriptional promoter used in compositions, systems, and methods disclosed herein can be a human PPARγ1 promoter, or a functional fragment thereof, such as a core or minimal human PPARγ1 promoter. A transcriptional promoter used in compositions, systems, and methods disclosed herein can be a mammalian PPARγ2 promoter, or a functional fragment thereof, such as a core or minimal mammalian PPARγ2 promoter. A transcriptional promoter used in compositions, systems, and methods disclosed herein can be a human PPARγ2 promoter, or a functional fragment thereof, such as a core or minimal human PPARγ2 promoter.

In some embodiments, a PPARγ promoter comprises about 100, about 200, about 500, about 1000, about 1500, about 1600, about 1700, about 1800, about 1900, about 2000, or about 2500 base pairs upstream of the human PPARγ transcriptional start site. In some embodiments, a PPARγ promoter comprises at least about 100, at least about 200, at least about 500, at least about 1000, at least about 1500, at least about 1600, at least about 1700, at least about 1800, at least about 1900, at least about 2000, or at least about 2500 base pairs upstream of the human PPARγ transcriptional start site. In some embodiments, a polynucleotide construct or expression regulatory region disclosed herein comprises or utilizes a PPARγ enhancer element. Illustrative PPARγ promoters or fragments thereof are provide in SEQ ID NOs: 58-60. In some embodiments, a PPARγ is as described in Zhu et al. “Structural organization of mouse peroxisome proliferator-activated receptor gamma (mPPAR gamma) gene: alternative promoter use and different splicing yield two mPPAR gamma isoforms.” Proceedings of the national academy of sciences 92.17 (1995): 7921-7925, as described in Fajas et al. “The organization, promoter analysis, and expression of the human PPARγ gene.” Journal of Biological Chemistry 272.30 (1997): 18779-18789, or as deposited under GenBank AF012873.1 or AF012874.1, each of which is incorporated herein by reference for such disclosure.

A transcriptional promoter used in compositions, systems, and methods disclosed herein can be a CD36 promoter, or a functional fragment thereof, such as a core or minimal CD36 promoter. CD36, also known as fatty acid translocase, is a multifunctional membrane glycoprotein involved in various cellular processes including fatty acid uptake, cell adhesion, and acting as a class B scavenger receptor. A study using the Genotype-Tissue Expression (GTEx) database categorized CD36 as an adipose-enhanced gene, indicating its expression is more than 5-fold higher in adipose tissue compared to most other tissues.

A transcriptional promoter used in compositions, systems, and methods disclosed herein can be a mammalian CD36 promoter, or a functional fragment thereof, such as a core or minimal mammalian CD36 promoter. A transcriptional promoter used in compositions, systems, and methods disclosed herein can be a human CD36 promoter, or a functional fragment thereof, such as a core or minimal human CD36 promoter. In some embodiments, a CD36 promoter comprises about 100, about 200, about 500, about 1000, about 1500, about 1600, about 1700, about 1800, about 1900, about 2000, or about 2500 base pairs upstream of the human CD36 transcriptional start site. In some embodiments, a CD36 promoter comprises at least about 100, at least about 200, at least about 500, at least about 1000, at least about 1500, at least about 1600, at least about 1700, at least about 1800, at least about 1900, at least about 2000, or at least about 2500 base pairs upstream of the human CD36 transcriptional start site. In some embodiments, a polynucleotide construct or expression regulatory region disclosed herein comprises or utilizes a CD36 enhancer element. Illustrative CD36 promoters or fragments thereof are provide in SEQ ID NOs: 64 and 65. In some embodiments, a CD36 promoter is as deposited in GenBank AF266759.1, as described in Armesilla and Vega. “Structural organization of the gene for human CD36 glycoprotein.” Journal of Biological Chemistry 269.29 (1994): 18985-18991, or as described in Zingg et al. “Novel 5′ exon of scavenger receptor CD36 is expressed in cultured human vascular smooth muscle cells and atherosclerotic plaques.” Arteriosclerosis, thrombosis, and vascular biology 22.3 (2002): 412-417, each of which is incorporated herein by reference for such disclosure.

A transcriptional promoter used in compositions, systems, and methods disclosed herein can be a Lipoprotein Lipase (LPL) promoter, or a functional fragment thereof, such as a core or minimal LPL promoter. Lipoprotein Lipase (LPL) is a central enzyme in lipid metabolism, predominantly functioning in the hydrolysis of triglycerides in chylomicrons and very low-density lipoproteins (VLDL) into free fatty acids and glycerol, crucial for lipid clearance, utilization, and storage in the body. In adipose tissue, LPL is key for lipid uptake, adipocyte differentiation, and maturation, acting as the main enzyme for the entry and reesterification of free fatty acids. Its regulation is complex and varies in response to physiological stimuli like nutritional changes, being highly responsive to fasting and exercise.

A transcriptional promoter used in compositions, systems, and methods disclosed herein can be a mammalian LPL promoter, or a functional fragment thereof, such as a core or minimal mammalian LPL promoter. A transcriptional promoter used in compositions, systems, and methods disclosed herein can be a human LPL promoter, or a functional fragment thereof, such as a core or minimal human LPL promoter. In some embodiments, an LPL promoter comprises about 100, about 200, about 500, about 1000, about 1500, about 1600, about 1700, about 1800, about 1900, about 2000, or about 2500 base pairs upstream of the human LPL transcriptional start site. In some embodiments, an LPL promoter comprises at least about 100, at least about 200, at least about 500, at least about 1000, at least about 1500, at least about 1600, at least about 1700, at least about 1800, at least about 1900, at least about 2000, or at least about 2500 base pairs upstream of the human LPL transcriptional start site. In some embodiments, a polynucleotide construct or expression regulatory region disclosed herein comprises or utilizes an LPL enhancer element. Illustrative LPL promoters or fragments thereof are provided in SEQ ID NOs: 66 and 67. In some embodiments, an LPL promoter is as described in GenBank X68111.1, or as described by Enerback et al. “Characterization of the human lipoprotein lipase (LPL) promoter: evidence of two cis-regulatory regions, LP-α and LP-β, of importance for the differentiation-linked induction of the LPL gene during adipogenesis.” Molecular and cellular biology 12.10 (1992): 4622-4633, each of which is incorporated herein by reference for such disclosure.

A transcriptional promoter used in compositions, systems, and methods disclosed herein can be a leptin (LEP) promoter (also referred to as OB), or a functional fragment thereof, such as a core or minimal LEP promoter. The leptin gene (LEP), encoding the hormone leptin, is primarily expressed in adipocytes and plays a key role in regulating food intake and energy expenditure, primarily through the central nervous system. The human LEP (ob) gene promoter, responsible for leptin expression, can require only 217 bp of 5′ sequence for basal adipose tissue-specific expression, with the CCAAT-enhancer-binding-protein-alpha (C/EBPα) site within this proximal promoter playing a crucial role in high-level expression in preadipocytes and adipocytes.

A transcriptional promoter used in compositions, systems, and methods disclosed herein can be a mammalian LEP promoter, or a functional fragment thereof, such as a core or minimal mammalian LEP promoter. A transcriptional promoter used in compositions, systems, and methods disclosed herein can be a human LEP promoter, or a functional fragment thereof, such as a core or minimal human LEP promoter. In some embodiments, a minimal LEP promoter (e.g., the about 217 bp of 5′ sequence) is used in a composition, method, or system of the disclosure. In some embodiments, a polynucleotide construct or expression regulatory region disclosed herein comprises or utilizes a CCAAT-enhancer-binding-protein-alpha (C/EBPα) site, for example, from within the LEP promoter. In some embodiments, while the leptin promoter can drive gene expression in vivo, it is not solely responsible for the adipose tissue specificity of leptin expression. A 30 bp region approximately 4.5 kb upstream of the LEP transcriptional start site has been identified as an enhancer in mature adipocytes, suggesting that multiple regions, particularly at the 3′ end of the gene, may contribute to its regulation. In some embodiments, a polynucleotide construct or expression regulatory region disclosed herein comprises or utilizes a LEP enhancer element (e.g., the approximately 30 base pair enhancer from approximately 4.5 kb upstream of the LEP transcriptional start site).

In some embodiments, a LEP promoter comprises about 100, about 200, about 500, about 1000, about 1500, about 1600, about 1700, about 1800, about 1900, about 2000, or about 2500 base pairs upstream of the human LEP transcriptional start site. In some embodiments, a LEP promoter comprises at least about 100, at least about 200, at least about 500, at least about 1000, at least about 1500, at least about 1600, at least about 1700, at least about 1800, at least about 1900, at least about 2000, or at least about 2500 base pairs upstream of the human LEP transcriptional start site. In some embodiments, a polynucleotide construct or expression regulatory region disclosed herein comprises or utilizes a LEP enhancer element. An illustrative LEP promoter or fragment thereof is provide in SEQ ID NO: 68. In some embodiments, a LEP promoter is as deposited in GenBank U48621.1, or as described in Miller et al. “The adipocyte specific transcription factor C/EBPalpha modulates human ob gene expression.” Proceedings of the National Academy of Sciences 93.11 (1996): 5507-5511, each of which is incorporated herein by reference for such disclosure.

A transcriptional promoter used in compositions, systems, and methods disclosed herein can be a Cell death-inducing DFFA-like effector C (CIDEC) promoter, or a functional fragment thereof, such as a core or minimal CIDEC promoter. CIDEC, also called FSP27, is a protein associated with lipid droplets in adipocytes, playing a crucial role in lipid droplet formation and potentially in adipocyte apoptosis. It's a member of the cell death-inducing DNA fragmentation factor-like effector family, important in apoptosis, and is regulated by insulin, correlating positively with insulin sensitivity. CIDEC expression is undetectable in 3T3-L1 preadipocytes but dramatically increases in mature adipocytes (15,000-fold), paralleling the expression patterns of PPARγ2. Analyzing 65 human tissues, CIDEC is predominantly expressed in mature adipocytes, with the highest expression in subcutaneous adipose tissue, followed by adipose tissue of unspecified origin and the omentum. This adipose-restricted expression pattern has also been shown through RT-PCR analysis of isolated adipocytes, adipose tissue, and various human tissues, showing that CIDEC expression is largely confined to adipocytes within adipose tissue. Comparative microarray and semi-quantitative PCR analyses identified CIDEC as one of the few adipose-specific genes in humans and mice, with higher expression in subcutaneous than in visceral adipose tissue. The 2.5 kb of 5′-flanking sequence of the CIDEC gene confers adipocyte-specific expression, with specificity achievable with only 176 bp of 5′ flanking sequences. However, in some embodiments a higher expression level is achieved with about 1,950 bp of upstream flanking sequence, indicating additional enhancer-like elements.

A transcriptional promoter used in compositions, systems, and methods disclosed herein can be a mammalian CIDEC promoter, or a functional fragment thereof, such as a core or minimal mammalian CIDEC promoter. A transcriptional promoter used in compositions, systems, and methods disclosed herein can be a human CIDEC promoter, or a functional fragment thereof, such as a core or minimal human CIDEC promoter. In some embodiments, various upstream and fragments of the human CIDEC gene can be used as a promoter or as part of an expression regulatory region in a composition, system, or method disclosed herein (which in some embodiments include some bases past the transcriptional start site), for example, about −1,800/+21, about −1561/+217, or about −269/−1 fragments.

In some embodiments, a CIDEC promoter comprises about 100, about 176, about 200, about 500, about 1000, about 1500, about 1600, about 1700, about 1800, about 1900, about 1950, about 2000, about 2100, about 2200, about 2500, or about 4500 base pairs upstream of the human CIDEC transcriptional start site. In some embodiments, a CIDEC promoter comprises at least about 100, at least about 200, at least about 500, at least about 1000, at least about 1500, at least about 1600, at least about 1700, at least about 1800, at least about 1900, at least about 2000, or at least about 2500 base pairs upstream of the human CIDEC transcriptional start site. In some embodiments, a polynucleotide construct or expression regulatory region disclosed herein comprises or utilizes a CIDEC enhancer element. An illustrative CIDEC promoter or fragment thereof is provide in SEQ ID NO: 71. In some embodiments, a CIDEC promoter is as described in Danesch and Ringold. “Cloning and transcriptional regulation of a novel adipocyte-specific gene, FSP27. CAAT-enhancer-binding protein (C/EBP) and C/EBP-like proteins interact with sequences required for differentiation-dependent expression.” Journal of Biological Chemistry 267.10 (1992): 7185-7193, Tan et al. “TNF-α downregulates CIDEC via MEK/ERK pathway in human adipocytes.” Obesity 24.5 (2016): 1070-1080, Chen et al. “FTO promotes SREBP 1c maturation and enhances CIDEC transcription during lipid accumulation in HepG2 cells.” Biochimica et Biophysica Acta (BBA)-Molecular and Cell Biology of Lipids 1863.5 (2018): 538-548, or Yasumoto et al. “Hepatitis B virus prevents excessive viral production via reduction of cell death-inducing DFF45-like effectors.” Journal of General Virology 98.7 (2017): 1762-1773, each of which is incorporated herein by reference for such disclosure.

A transcriptional promoter used in compositions, systems, and methods disclosed herein can be a Tumor suppressor candidate 5 (TUSC5) promoter, or a functional fragment thereof, such as a core or minimal TUSC5 promoter. TUSC5 is a cold-repressed gene initially identified in brown adipose tissue (BAT) transcriptome analyses and found to be robustly expressed in mouse white adipose tissue (WAT) and BAT, as well as in human adipocytes. It significantly increases during adipogenesis and is a target gene of PPARγ, which binds to its promoter region. TUSC5 regulates insulin-stimulated glucose uptake and transport in adipose tissue by modulating GLUT4 recycling. Its expression correlates with improved insulin sensitivity in obese patients. TUSC5 is identified as an adipose-specific gene, highly expressed in specific fat tissues and minimally in other tissues.

A transcriptional promoter used in compositions, systems, and methods disclosed herein can be a mammalian TUSC5 promoter, or a functional fragment thereof, such as a core or minimal mammalian TUSC5 promoter. A transcriptional promoter used in compositions, systems, and methods disclosed herein can be a human TUSC5 promoter, or a functional fragment thereof, such as a core or minimal human TUSC5 promoter. In some embodiments, a fragment about 200, about 500, about 1000, about 1200, about 1400, about 1600, or about 1800 base pairs upstream of the Tusc5 gene, for example, a −1415/+276 fragment. In some embodiments, a TUSC5 promoter used as a promoter or as part of an expression regulatory region in a composition, system, or method disclosed herein can include bases from upstream and/or downstream of the TUSC5 transcriptional start site, e.g., a −1415/+276 fragment. In some embodiments, a TUSC5 promoter comprises about 100, about 200, about 500, about 1000, about 1500, about 1600, about 1700, about 1800, about 1900, about 2000, or about 2500 base pairs upstream of the human TUSC5 transcriptional start site. In some embodiments, a TUSC5 promoter comprises at least about 100, at least about 200, at least about 500, at least about 1000, at least about 1500, at least about 1600, at least about 1700, at least about 1800, at least about 1900, at least about 2000, or at least about 2500 base pairs upstream of the human TUSC5 transcriptional start site. In some embodiments, a polynucleotide construct or expression regulatory region disclosed herein comprises or utilizes a TUSC5 enhancer element. An illustrative TUSC5 promoter or fragment thereof is provide in SEQ ID NO: 72.

A transcriptional promoter used in compositions, systems, and methods disclosed herein can be a Cell death-inducing DNA fragmentation factor alpha-like effector A (CIDEA) promoter, or a functional fragment thereof, such as a core or minimal CIDEA promoter. CIDEA is a lipid droplet-associated protein in adipocytes, regulating triglyceride deposition. CIDEA can be highly expressed in human white and brown adipose tissue, with its expression influenced by body fat status. In some embodiments, CIDEA expression decreases in obesity and correlates inversely with metabolic syndrome characteristics. CIDEA as an adipose-specific gene in humans.

A transcriptional promoter used in compositions, systems, and methods disclosed herein can be a mammalian CIDEA promoter, or a functional fragment thereof, such as a core or minimal mammalian CIDEA promoter. A transcriptional promoter used in compositions, systems, and methods disclosed herein can be a human CIDEA promoter, or a functional fragment thereof, such as a core or minimal human CIDEA promoter. In some embodiments, a CIDEA promoter comprises about 100, about 200, about 500, about 1000, about 1500, about 1600, about 1700, about 1800, about 1900, about 2000, or about 2500 base pairs upstream of the human CIDEA transcriptional start site. In some embodiments, a CIDEA promoter comprises at least about 100, at least about 200, at least about 500, at least about 1000, at least about 1500, at least about 1600, at least about 1700, at least about 1800, at least about 1900, at least about 2000, or at least about 2500 base pairs upstream of the human CIDEA transcriptional start site. In some embodiments, a polynucleotide construct or expression regulatory region disclosed herein comprises or utilizes a CIDEA enhancer element. An illustrative CIDEA promoter or fragment thereof is provide in SEQ ID NO: 69. In some embodiments, a CIDEA promoter is as disclosed in Pettersson, et al. “Characterization of the human CIDEA promoter in fat cells.” International journal of obesity 32.9 (2008): 1380-1387, which is incorporated herein by reference for such disclosure.

A transcriptional promoter used in compositions, systems, and methods disclosed herein can be a Lipase E (LIPE) promoter, or a functional fragment thereof, such as a core or minimal LIPE promoter. LIPE, also known as hormone-sensitive lipase (HSL), is highly expressed in adipose tissue and can be important for adipose tissue lipolysis, releasing free fatty acids for energy use. LIPE has been identified as an adipose-specific gene expressed in subcutaneous fat tissue.

A transcriptional promoter used in compositions, systems, and methods disclosed herein can be a mammalian LIPE promoter, or a functional fragment thereof, such as a core or minimal mammalian LIPE promoter. A transcriptional promoter used in compositions, systems, and methods disclosed herein can be a human LIPE promoter, or a functional fragment thereof, such as a core or minimal human LIPE promoter. In some embodiments, a LIPE promoter comprises about 100, about 200, about 500, about 1000, about 1500, about 1600, about 1700, about 1800, about 1900, about 2000, or about 2500 base pairs upstream of the human LIPE transcriptional start site. In some embodiments, a LIPE promoter comprises at least about 100, at least about 200, at least about 500, at least about 1000, at least about 1500, at least about 1600, at least about 1700, at least about 1800, at least about 1900, at least about 2000, or at least about 2500 base pairs upstream of the human LIPE transcriptional start site. In some embodiments, a polynucleotide construct or expression regulatory region disclosed herein comprises or utilizes a LIPE enhancer element. An illustrative LIPE promoter or fragment thereof is provide in SEQ ID NO: 70. In some embodiments, a LIPE promoter is as deposited in GenBank AJ222693.1 or as described by Grober et al. “Characterization of the promoter of human adipocyte hormone-sensitive lipase.” Biochemical Journal 328.2 (1997): 453-461, each of which is incorporated herein by reference for such disclosure.

In some embodiments, a transcriptional promoter is an Ap2 or UCP1 (uncoupling protein 1) promoter, or a functional fragment thereof.

TABLE 1 provides sequences of illustrative promoter elements.

TABLE 1

| SEQ | | |
|---|---|---|
| ID | | |
| NO | Description | Sequence |
| 1 | Adiponectin | AGCTTTAAGAATTCAGGGCCTTTTTAACTTGCCAA |
| | promoter | GCCCCACACCACTCCAGGAACTTCCCCACACCCCA |
| | fragment | GTTCTCAGAATTCATGTGCAAGGTCTTTCCTAAAT |
| | | CCAGGGTCCAGGTCAGAGAGTGGAGGATGTGCTCT |
| | | ATTTCTTACCTGATTGCAGACCCCTCTGACAGTGC |
| | | TCCCTTCTGAAGCACTCACTGTCTGAACGTACACA |
| | | GTCTCAGACTTAATCATGCACAGTGAGCAAGACTG |
| | | TGGTGTGATAATTGGCGTCCCTGACTTATTAGGGC |
| | | AAATCTATGGGAGGGGGAGACCTCCTGGACCACTG |
| | | AGCAATTAATTCATTTACATTAGGAAGTTTCTCCG |
| | | TCAGATGCAGGAAAAAAATCTTGTTTTCCTGCTGT |
| | | GGTTTTGACTTTTGCCCCATCTTCTGTTGCTGTTG |
| | | TAGGAGGCAAAATAAGGGTCAAGGCCTGGAAACAC |
| | | AAGTGCTTTGACTGAAGCTCCACTTGGCTTCCGAA |
| | | GCCCAAGCTGGGTTGTACCAGGTTCCCTAGGGTGC |
| | | AGGCTGTGGGCAACTGCCAGGGACATGTGCCTGCC |
| | | CACCGGCCTCTGGCCCTCACTGAGTTGGCCAATGG |
| | | GAAATGACAATTGTGAGGTGGGGACTGCCTGCCCC |
| | | CGTGAGTACCAGGCTGTTGAGGCTGGGCCATCTCC |
| | | TCCTCACTTCCATTCTGACTGCAGTCTGTGGTTC |
| 56 | PLIN1 promoter | AATGTTTTCCACCCCAGAGCCTCCACATTCATGAG |
| | fragment | GGATGCCAAAAATCGAAGAAGTTCTTCGTAGATGT |
| | | ACAGCCCAGCACATTCACAACTGAGCATTGTTGCC |
| | | TGACAATTCATTTATTCAACAATGAATGTTTGAGT |
| | | GCCATGCATGGTTTTGGGTATCCACTTGTGAGCAA |
| | | GATGACCTAGGTCCCTGCTCTCACTTGGTGGAGGG |
| | | GTAGGGAAAGCAGGTAGGTACGGAGAAGGAAAGAA |
| | | GATAGAACTCTATGCCCTGCAAGCTACTGCTAGAA |
| | | AACTAACACTTCTGTCTTCCCTCGGGACTCTCCTA |
| | | GGCCCTTTCACCACTTCTCACCTCTCACCCACATT |
| | | CTGCACTCCCAGCTAAAGCAGCCAAAGGACTATTT |
| | | GATTTGGCAGAGCTTTCAGGCACAAAGCCCTATGT |
| | | TCCCTCTGGCCCAAAGCCACTACTCTGAAGGCTGA |
| | | AAACTGCCCTTTGGCTCAGGTTGCTGTCCAGTCAC |
| | | AGCCTCACAGATAATTTGTGCTGAACCTTGAGGTG |
| | | AGGGCAGAGGCCACTTCTTTAAATACTTTGTCTTC |
| | | TCTACTGCCTTACATTTGAAAAGCACAGGTTGGAT |
| | | CAATAAGTATTAAACGTATAGCGTATTACTCCTTT |
| | | GAAGCCTCCCTGCTTGACTCCTCCCTTTCCTGCCT |
| | | TCTCATTCCCTACTTTATTGCATCACAGATACGTA |
| | | ATTTATTACCAAGGATCTTTCATAATTCCCCACCC |
| | | CTGCCTCTTTCTTCTCTTGCCCACACTGTGTGACT |
| | | TAACAGAGTCAACAATACATATAATTCCACCAGCA |
| | | ACAAAGAAAATAAGTTTTGTTTTCCTATTGTCTGT |
| | | TTTCATGAAGAGGCCCTTCTTAGTTAATCTGAAAG |
| | | CTATGGCTTATACCATAGTCCTCCATCATATCTAA |
| | | AGCCAGGAACTAGAACTTGGGCTTGTGCTTGAGTC |
| | | CTGGGTAAAGAAGGGTTGAGTGGTGCTAATTCTTG |
| | | GCTGTCTGGGTGCTCTTGATTAGTGATAGAATCCT |
| | | AGTCAACACTGTCGCTTATCCAGACCCAGGAGAAT |
| | | AAAAATCTACTTGGCTCTCCTACTCTTCATTCTGG |
| | | TAGCATCTTAGGCCAGGTAAAGTTCAATAAATTTT |
| | | TTTGAATTCTAAGAAGTGGGAATTCCTCCCTCAAC |
| | | TCAAAGGCTATATTGTCAAAAACTCAACTCCCGGC |
| | | TCTGTCGCTTCTTCACTGAGGGCAATTTATCTCCA |
| | | TAATCAAAGATGAATACATTCAGATGAACTCTTCT |
| | | GTGTGGGTTTAAATTTTACTGTTCAGCATTCAGTC |
| | | TTGCTAGTAAATAAGAAAAGATGAGAGAAATGGAT |
| | | TGAAAACTCCCACAAGACCCTCTATGGAAAAGGAG |
| | | CCAAAAGATTGCAGAGGTATATAGAACTTTAGAGC |
| | | TTTTGAGTTTAATGATGTCTATCCCTATACCTTCT |
| | | TTCTGGAAAGCAGATTTTTGTGAATTGAGGGCAGG |
| | | AACATTACAGAACTGATGCTTCATGGCTCTGGAAT |
| | | ACTAAGTCCTCTGAGCCTGGATCTCCTTCAGCTCT |
| | | GCCACCTACCTCCTTGTTTGCTAAATGTCCTTAGA |
| | | GAAGTTAGGATATGCCTGGGGAACCCTGGGTCATA |
| | | TAACCATGACAACTGGCTGACCCTGTTGTCTCTCC |
| | | CTCTCTGCCCTCCTCTAGCTGGGAGGTGGAAGCAG |
| | | CATTGCCCAAGCCTCCCAGGAGTGACAGGAATTGT |
| | | TTCTGCCTGAGGAGACA |
| 57 | PLINI promoter | TCCCTACTTTATTGCATCACAGATACGTAATTTAT |
| | fragment | TACCAAGGATCTTTCATAATTCCCCACCCCTGCCT |
| | | CTTTCTTCTCTTGCCCACACTGTGTGACTTAACAG |
| | | AGTCAACAATACATATAATTCCACCAGCAACAAAG |
| | | AAAATAAGTTTTGTTTTCCTATTGTCTGTTTTCAT |
| | | GAAGAGGCCCTTCTTAGTTAATCTGAAAGCTATGG |
| | | CTTATACCATAGTCCTCCATCATATCTAAAGCCAG |
| | | GAACTAGAACTTGGGCTTGTGCTTGAGTCCTGGGT |
| | | AAAGAAGGGTTGAGTGGTGCTAATTCTTGGCTGTC |
| | | TGGGTGCTCTTGATTAGTGATAGAATCCTAGTCAA |
| | | CACTGTCGCTTATCCAGACCCAGGAGAATAAAAAT |
| | | CTACTTGGCTCTCCTACTCTTCATTCTGGTAGCAT |
| | | CTTAGGCCAGGTAAAGTTCAATAAATTTTTTTGAA |
| | | TTCTAAGAAGTGGGAATTCCTCCCTCAACTCAAAG |
| | | GCTATATTGTCAAAAACTCAACTCCCGGCTCTGTC |
| | | GCTTCTTCACTGAGGGCAATTTATCTCCATAATCA |
| | | AAGATGAATACATTCAGATGAACTCTTCTGTGTGG |
| | | GTTTAAATTTTACTGTTCAGCATTCAGTCTTGCTA |
| | | GTAAATAAGAAAAGATGAGAGAAATGGATTGAAAA |
| | | CTCCCACAAGACCCTCTATGGAAAAGGAGCCAAAA |
| | | GATTGCAGAGGTATATAGAACTTTAGAGCTTTTGA |
| | | GTTTAATGATGTCTATCCCTATACCTTCTTTCTGG |
| | | AAAGCAGATTTTTGTGAATTGAGGGCAGGAACATT |
| | | ACAGAACTGATGCTTCATGGCTCTGGAATACTAAG |
| | | TCCTCTGAGCCTGGATCTCCTTCAGCTCTGCCACC |
| | | TACCTCCTTGTTTGCTAAATGTCCTTAGAGAAGTT |
| | | AGGATATGCCTGGGGAACCCTGGGTCATATAACCA |
| | | TGACAACTGGCTGACCCTGTTGTCTCTCCCTCTCT |
| | | GCCCTCCTCTAGCTGGGAGG |
| 58 | PPARγ1 | CCCCTGCCCCTGCCCCTGCCCCCACCCCCACCCCC |
| | promoter | ACCCCCACCCCCAGCCGGCGCCCGCGCCCGCCCCC |
| | fragment | GCGCCGGGCCCGGCTCGGCCCGACCCGGTTCCGCC |
| | | GCGGGCAGGCGGGGCCCAGCGCACTCGGAGCCCGA |
| | | GCCCGAGCCGCAGCCGCCGCCTGGGGCGCTTGGGT |
| | | CGGCCTCGAGGACACCGGAGAGGGGCGCCACGCCG |
| | | CCGTGGCCGGCAGATTTGAAAGAAGCCGACACTAA |
| | | ACCACCAATATACAACAAGGCCATTTTGTCAAACG |
| | | AGAGTCAGCCTTTAACGAAATGACCATG |
| 59 | PPARγ2 | CAGTAGCATGCTGATACCAACGTTTAAACTATGGA |
| | promoter | TACATATTTGAATTCCAAATTTTTCTTCAGATAAT |
| | fragment | GTGATTAGAGATTAGAGATTCAACCAGGGATAGAC |
| | | ACCGAAAGAAAACTTTGCCCAAATAAGCTTTCTGG |
| | | TATTTCATAAGCAAGAGATTTAAGTTTTCCATTTA |
| | | AGAAGCCATTGTGAATTATACAACAATAAAAAATG |
| | | CAAGTGGATATTGAACAGTCTCTTCTCTGATAATT |
| | | CTAAATACAGTACAGTTCACGCCCCTCACGAGACA |
| | | CTGAACATGTGGTCACCGGCGAGACAGTGTGGCAA |
| | | TATTATCCCTGTAATGTACCAAGTCTTGCCAGAGC |
| | | AGTGAACATTATGACACAACTTTTTGTCACAGCTG |
| | | GCTCCTAATAGGACAGTGCCAGCCAATTCAAGCCC |
| | | AGTCCTTTCTGTGTTTATTCCCATCTCTCCCAAAT |
| | | ATTTGGAAACTGATGTCTTGACTCATGGGTGTATT |
| | | CACGATTCTGTTACTTCAAGTCTTTTTCTTTTAAC |
| | | GGATTGATCTTTTGCTAGATAGAGACAAATATCAG |
| | | TGTGAATTACAGCAAACCCATATTCCATGCTGTTA |
| | | TG |
| 60 | PPARγ promoter | GAGGATCACTTGAGCCCAGGAGTTTCAGACTAGTC |
| | fragment | TAGGCAACATGTCAAGACACAGTCTCTACTAAACA |
| | | ATTAAAATTAAAAAAAAAAATTAGCCAGGCATGGT |
| | | GGTGTGCACCTGTAGTCCCAGCTACTTGGGAGCCT |
| | | GGGGTGGGAGGATTCCTTGAGCCCGGGAAGTCGAG |
| | | GCTGAAGTGAGCCGTGATTGCGCCACAGCACTCCA |
| | | GCCTGGGCGACACAGCAACACCCTGTCTCATGGAA |
| | | GAAAGAAAGAAAAGAAAGGAAGAAAGAAAAAAAAA |
| | | AAGCAGATTGGAACTCTGGAATTAACAAGAAGTAG |
| | | GACGCACGGAGCACTTCCGCCTGAGTGGAGACTGT |
| | | GGATCCGGGTCAACCTGACTACCTAAATCACAGGC |
| | | CAATAAATGGTCTTTCAGTGGTCAGTCCCTGTAAG |
| | | ATCCGTGGCTCTCAGCTTCTTATCTTAGGGGCTGT |
| | | GGAGGAAGGACATGATTATGTTGATTTAAGCGCTG |
| | | AATATTTTCCCTTGTGATACCCATCCTCGCAAAAC |
| | | TTTGCTTCAACCACAAACGAGGACCTTCTGTACCA |
| | | GAGGGGCAATAACCACAATGAAGCTAGGAAGAAAT |
| | | GCAGAGCACCCCAGCATACAGTCCATAAGCTTCCT |
| | | GAAGTGGGGGGCCTCAGGCATCGCTGCCTCCCCAA |
| | | AGAGGATCAGGCCCAGAACAGTATGCTCCAGAAAT |
| | | AAGACTGGAAAAAGGGAAAGAGGGGCCTCAAGTCC |
| | | AGGAGACCAGCGGCTTTCTGAACGCGCACCTGCCA |
| | | ACCCACTTTGGACAGGTCACGATGGACAGCGTGGC |
| | | AGGAAAAGAAAAGGTCACTGTCTACCCAACACATG |
| | | AGAAACTGTTTCTCGTGCCTCACGTCCCCACTCCG |
| | | TCCCCACCCATGTTGTCTGAGTCCCTCGGTGTCAG |
| | | AAACACTGCTAAGAAATTTAAGAAATTCTGTTAAT |
| | | GAGTTTAAGAAATGTTTTTAATGATTAAAAGTCAG |
| | | TGACTTGTGAATAACCATGT |
| 61 | aP2/FABP4 | CAGTGGGATACACAGTGTAGCGATGCATCACTCTG |
| | promoter | AAATATTTTAGTTTCTTTTTTTCCCCTAAATCTGG |
| | | GTATGTTCGTGGGAATTTGCAGCACATGTGAACAA |
| | | CTTCTGTCATTCTTGCATGAGGCAAAGGGAATTGA |
| | | AAACCACGATTACTTTAGAAAACTAGTTTCACAGA |
| | | TTGGTCACTGTATAAAAGAAGGATATTGGTTTTGG |
| | | TAGCTTGTGACCACACACCATTTCTGATCTGAATA |
| | | AATTCAGAACTTATAATACAGTTCAGAAATTGAAT |
| | | GCAGTTTCTCAATATGAGGAAAGTATTTTAGAATA |
| | | AGGCCTATTTTTCAAAGGATCTGTGGAAATCAATG |
| | | CTATGCTCTCATTTAGGAGATGGAAAGAGTGAGGT |
| | | TAAATTATCATTTCGATTAAATCTACAGTCCAGAT |
| | | TACTGGTGGATGAATTGAATGTACTTTTTTATTCA |
| | | TATAAAACATTTGAAATCAGAAATCTGGAGTACTT |
| | | TTAAATCCCATTATTTATTTTGTTTTAATCGCCAG |
| | | GTAATTCCTGAGACAGGAGTGTCCCGAAGAGCCTT |
| | | TGCAATTATGTAAGAATCTCCGAGGCAGTTCTTAT |
| | | GTTCCTCAATTCAAAAGAACCACATAACTGCAATT |
| | | TAAATAACACCCCACACACACACACAAAATAAGGT |
| | | CGAAGTTTATCTCAAAATAATTTCCCCTCTCTACA |
| | | CTGGGATAAATATGTATAGGAATAATAGGGGGAAA |
| | | TTCAGTGCACTGAGCATTAAGCTGTCAAAACAGGA |
| | | ATGTTTAAAATATCCTGTTAGTGGTTTAAAAATAA |
| | | TTTGTACTCTAAGTCCAGTGACTATTTGCCAGGGA |
| | | GAACCAAAGTTGAGAAATTTCTATTAAAAACATGA |
| | | CTCAGAGAAAAAAATGCAGAGGCCGGTAATGAAGG |
| | | AAATGATTGGATCTCATTCCCAATTGGTCATTCCT |
| | | AAGATCACATGTTCTGAGCATCTTTAAAAGGAAGT |
| | | TATCTGGACTCAAGAGGGTC |
| 62 | aP2/ | AGGAGAGCCTTGGCATGATCCTTAACCTATGACAT |
| | FABP4 | ACATGCTGTGAAAACACTCCCCCTCATCAGGGCTT |
| | promoter | TGTCCCTACTCCTCATCATCCAGAGAGAGGGTATG |
| | | TTTCCTCTTCAACCTAAAAATTGTCAGAAAAAATG |
| | | CTCTCATTTCCTCCAAAAGAAATAAACACAGGCAA |
| | | AGGTCAGAGGGATGCATTCCATGGAAGCTGTCCAT |
| | | AGGAGAGCAAAGTGCAAGGCAGACGGCCTGAGCCA |
| | | GGGCTATGAAGCACTGAGGGTCTGGGGCTATGTCC |
| | | CCAGTCTCCACATCCCCAAAGAAAAGCAAAACTCT |
| | | GGCTACTGAAGACCCCTTCCCACAGCCCTAAGGAA |
| | | ACATTCTGACCTGGTGTGTGCATGTCTATGCTGTA |
| | | CTGGGGTACGTGTGAATAAAAGTATAGTCACCTCT |
| | | GTGGGAATTCTTTTCATAGTCAAAGGCAAATTGTC |
| | | TCCTATGCACCTTGTTTCTGAGTACTAAGCCCCTC |
| | | CAAAAGGGAAGGAGTGTCCTATGTACTTTCTATAT |
| | | TGTTTCTCAATATCTTTCACTGCTACCCCATTGTC |
| | | TCATTTCATTGCCCTTGCCCAGCCCCCATCATCTC |
| | | TTAACTCCTGCCTTGTGAGCAGATTTACATCTGCT |
| | | CACTCTATCCTTTATAATCCACTCTGCCTAGGGCC |
| | | ACTGTATTCATTGTTCTAAAACTGAAATAGGAGCC |
| | | CATGGGGGGTTTTAAACACATATACAAGCATTAAG |
| | | CATGAATATGTAGTTTAAATCCGTATCTTATTCCT |
| | | TTTGTTAATTATTTATTAATTCAAGTATTTGGTGA |
| | | AGAACCAGGTGCCAGGAACTTTAAGTCATGCATGG |
| | | GATAGAGTTTGGGATGGAGAAGGGAAGTCAGGAAT |
| | | GAGGATAGATCGGGAATGGGATGAATGTATCTCTC |
| | | ATCTTGTATACATTACATGCTAATGAAAGGTGATA |
| | | AAAGTGTAAAAAAAATTGGAAGAGCTACTAGTGCC |
| | | TTCTGTACAAAATATGCTAATATCAAGAAGACTAT |
| | | CAAAACAAAACACCTTTTAAAATGCTTTGTATGTT |
| | | ATTTTGGTCCCAGGCTGGTAACTAGAATGAGAGCA |
| | | TAGATTACAAAAGAGAGACACAAATTATGTCTTAT |
| | | TTATTATTATATTCCCTTACCCCTATGTAGTACAG |
| | | TGCCTTAAGCATAACAGGCTTGCAAAAAAAGATTT |
| | | TTTGTAAATTATTTGCCAGCGTGCACTTTATACAA |
| | | TGAAACTATGTTTGAAAATGCCAATAGTATAATTT |
| | | CTACTAGTTTTCAAGAAAGGATGACCACCAAAATT |
| | | TTGGTGAAGTCCAGATTCTCTTCATTAATGCTTAG |
| | | TGAGGATTTCCTTCACAGAGCATTGAGGCCAAGGT |
| | | CGTATAATATTGAATTCGTTTTTTCATGTGATTCT |
| | | TCTTATTTTGTATTGCTTCATTCACATAGCAATCC |
| | | ATCAACATTGTGATGAAAGATGATAGTAATTTGAT |
| | | GAGTGAACATTCCATTTTCTCCATGTCGGCAAGAA |
| | | TAACTCTTAGGTATTAAGATAAAAATAATTTCTTT |
| | | AGGAGCTGAACAGTTGTTTACATTCCTGTTTATAC |
| | | TGCAGAGCAGGAGTTTTCGGCCTCTGCATTCAATT |
| | | TCTGGACAGCTTATCATCCAGAAAGAGTTCTCTAC |
| | | CCTACACGAGACAAAACCTAGTGGACCTTTGCATA |
| | | GTAAGAATATTAGTATTCCACAACATGTTCCATGT |
| | | GTTTAGGATATACACTGAGCTGCTTTTAAGGGCAA |
| | | TATCTCTCAGGTAAGAAACTCTGCCAAGCCTTGTT |
| | | ACTCTTAACAAGTGATCATATGAAAGGTCTTCCTG |
| | | TGGACTCAACTACCTTCTCACATCTTACTTCTCTC |
| | | ATTGCTGTTTAAGTTACCAAGATCCTTTACTATAA |
| | | GTTACTACTATTCTTGTTTATATGAACTCTAAAAG |
| | | AGCCAATAGTTGTTTCTAGAAAATCTCAACTACGC |
| | | CATTTCTCCAACGATGATCAGGCACTGAATATGCT |
| | | GGTATAGAAGATGAGGCTACTGGCCTACAAATACA |
| | | TTCTTCAAGGTTTTTTTTGTTGTTTTTTATAGGCG |
| | | TGCTGGTGGACTCTCACTAACCCGTCGAAGAAGGA |
| | | TTTCCTTCAGGCGTTTTACTGAATATATAATTACT |
| | | TTGTATCTAACAATGAATTAATCTGAACAAAAGTC |
| | | TCTTCCTTAATGGACCTTGGACTTTGCTCACCAGG |
| | | AACATATATCTGTATATGTCAGGGTGGCTGTGTTT |
| | | GGGTGAAATTGTTCTGAAAGGTAATGGAACCAGCA |
| | | GCTCCCGCAGTTTTCTGTCCCGTATACAGTCTCTT |
| | | TCCTGATCTTCCTGTGGCTGTCTTCTTCTCATCAT |
| | | TCAGCTAACAGCTCAATTATCTACCTTCTTTAACA |
| | | GACTTTCCCGGATTGCCCCATCTAGTGCTCCAACC |
| | | CTCACAGTGGTCATTACATCAGAGTACCCCATTCT |
| | | GTTTGCTCTAAAACACAGCACTACCTGTGATTGGC |
| | | TTATTTATTTCATATTTCATACACAAGGTCTCTTT |
| | | GTATATGACCTTCTCCAGTATTAGAATATAAGCTC |
| | | ATTGAGAGCAAGCAAATGCCTGCCCTGGCTGGCTC |
| | | ACTGTTGAATCCGCATTGGTTAGCACAATGCCTGA |
| | | TCACATGTTGAATAAATAAGCGAAGCTTGCTGTTA |
| | | CCATGGTACTCTAAAACTTCACTGCCCAAGAGAAA |
| | | TATAATGTAAGCCACATATATAATCTAAAATTTTC |
| | | TAGCAGCCACATAAGGAAAGTAAAAAAGAAATAGG |
| | | TTAAATTAATTTTCGTAATACATTCTATTTAACCT |
| | | AACATATCGAAAATGTTGTCATTTCAACATGTAAT |
| | | CAATGTAAGAAAATGAATAATATAGGATATATTTG |
| | | TATATTGTCTTCAAAATCTGGTATATATTTTATAC |
| | | TAACAGTTCATCTCAATTCAGATTCTAAATTTTCT |
| | | TTTGAAATACTTGTCCAACATTTAGATTTCATAAA |
| | | ATTTACAATTGAAAAAGTAGATTGACATAGCCACA |
| | | TTGGTTCAAACATATTTAAAAGTTTTTCAGTAACT |
| | | GAATTGAGGATCATTTCTTTCATTTTAATTAAAAT |
| | | TAAGTAAAATTAAAAATTCAATTTCTCAGTCATAC |
| | | TAGCCACATTTTAAGAGCCACATGTGACCGGAGAT |
| | | TACACTACCAGAGAGTCAATCTCTAAAACAATTAT |
| | | TCACAATAAAAAAGTCACGGAATCAACCTAAGTGT |
| | | CCATCAGTGGATGACTGGATGAAGACAATGTGATA |
| | | TATATTTACCATGGAATACCACTCAGCCATAAAAA |
| | | GGGATGAGATCATTTCTTTTGCAGCAACATAGGTG |
| | | AAACTGGAGACCATTATCTTAAGTGAAATAAATCA |
| | | GAAACAGAAAGTCAAATACCACATATTCTCACTTA |
| | | TAAGTGGGAGCTAAACAATGTGTACACACAGACGT |
| | | ACAGAGTGGCATAATAGACAATTGGAGACTCCAAA |
| | | AGGTGGGAAGGTGGGAAGGTGGGAGGGGGCAAGGG |
| | | ATAAGAAATTACCTATCAGGGCCGGGCACAGTGGC |
| | | TCACGCCTGTAATCCCAGCACTTTGGGAGACTGAG |
| | | GCGAGTGGGTCACCTGAGGTCAGGAGTTCAAGACC |
| | | AGCCTGGCCAACATGGCGAAACCCTGTCTCTACTA |
| | | AAAATATGAGAAAAAAAATTATCCAGGCATGTGGT |
| | | GGGCACCTGTAATCCCCGCTACTAGGGAGGCTGAG |
| | | GTAGGAGAATCGCTTGAACCTGGGAGGCAGAGGTT |
| | | GCAGTGAGCCAAGATCGCGCCACTGCATTCCAGCC |
| | | TGGACAATAAAATCAAAACTCCATCTCAAAAAAAA |
| | | AAAAAAAAGAAAGAAATTACCAATTGAGTACAATA |
| | | TACACTATTTGGGTGATGATTACACTAAAAGTCCA |
| | | GACTTTATTACAACTCAATATACATATGTAACAAA |
| | | AATGCACATGTAGTCCGAAATCTATAGAAACAAAA |
| | | ATTTTAAAAAACCAAATTGCAGTGTTAGAATACAC |
| | | ATAGTATGTATTCAAATTCAATCATTTAAAAACCT |
| | | GTAGGAAGCAAATTAATTTAAAACATTATCAAAAA |
| | | CATCCCTCTATTAAGATTTGTCACTTTTATTTACT |
| | | TTCCATCTGAACAGTCTTATTCAATGGGAAACAGG |
| | | TGGATCACTAATAGCAAGCCTACCTGGATGGTACA |
| | | TCACCACAGTCTGTGTATCTATGCAACAGTCCAGA |
| | | GTAGATGAGCGTTATTACTGTGCCATTAACAAATG |
| | | CAAAACCTAACTTTATCAGGCTTTCATAGCAACTT |
| | | GCTTTCCTACAGAAAATCCTGTTGCTTACAGAATT |
| | | TCCTGCCACAAACATTTTTGGGAAGCCCTCAGGAC |
| | | ACTTACACAGGCTTGTGAATCAATCACATGCTCAG |
| | | CAGGTACTGTTCAGTGGGATACACAGTGTAGCGAT |
| | | GCATCACTCTGAAATATTTTAGTTTCTTTTTTTCC |
| | | CCTAAATCTGGGTATGTTCGTGGGAATTTGCAGCA |
| | | CATGTGAACAACTTCTGTCATTCTTGCATGAGGCA |
| | | AAGGGAATTGAAAACCACGATTACTTTAGAAAACT |
| | | AGTTTCACAGATTGGTCACTGTATAAAAGAAGGAT |
| | | ATTGGTTTTGGTAGCTTGTGACCACACACCATTTC |
| | | TGATCTGAATAAATTCAGAACTTATAATACAGTTC |
| | | AGAAATTGAATGCAGTTTCTCAATATGAGGAAAGT |
| | | ATTTTAGAATAAGGCCTATTTTTCAAAGGATCTGT |
| | | GGAAATCAATGCTATGCTCTCATTTAGGAGATGGA |
| | | AAGAGTGAGGTTAAATTATCATTTCGATTAAATCT |
| | | ACAGTCCAGATTACTGGTGGATGAATTGAATGTAC |
| | | TTTTTTATTCATATAAAACATTTGAAATCAGAAAT |
| | | CTGGAGTACTTTTAAATCCCATTATTTATTTTGTT |
| | | TTAATCGCCAGGTAATTCCTGAGACAGGAGTGTCC |
| | | CGAAGAGCCTTTGCAATTATGTAAGAATCTCCGAG |
| | | GCAGTTCTTATGTTCCTCAATTCAAAAGAACCACA |
| | | TAACTGCAATTTAAATAACACCCCACACACACACA |
| | | AAATAAGGTCGAAGTTTATCTCAAAATAATTTCCC |
| | | CTCTCTACACTGGGATAAATATGTATAGGAATAAT |
| | | AGGGGGAAATTCAGTGCACTGAGCATTAAGCTGTC |
| | | AAAACAGGAATGTTTAAAATATCCTGTTAGTGGTT |
| | | TAAAAATAATTTGTACTCTAAGTCCAGTGACTATT |
| | | TGCCAGGGAGAACCAAAGTTGAGAAATTTCTATTA |
| | | AAAACATGACTCAGAGAAAAAAATGCAGAGGCCGG |
| | | TAATGAAGGAAATGATTGGATCTCATTCCCAATTG |
| | | GTCATTCCTAAGATCACATGTTCTGAGCATCTTTA |
| | | AAAGGAAGTTATCTGGACTCAAGAGGGTCACAGCA |
| | | CCCTCCTGAAAAC |
| 63 | Mini aP2 | GATTAACCCGCCATGCTACTTATCTACTCGACATT |
| | /FABP4 | GATTATTGACTAGGGGAATTCCAGCAGGAATCAGG |
| | promoter | TAGCTGGAGAATCGCACAGAGCCATGCGATTCTTG |
| | | GCAAGCCATGCGACAAAGGCAGAAATGCACATTTC |
| | | ACCCAGAGAGAAGGGATTGATGTCAGCAGGAAGTC |
| | | ACCACCCAGAGAGCAAATGGAGTTCCCAGATGCCT |
| | | GACATTTGCCTTCTTACTGGATCAGAGTTCACTAG |
| | | TGGAAGTGTCACAGCCCAAACACTCCCCCAAAGCT |
| | | CAGCCCTTCCTTGCCTTGTAACAATCAAGCCGCTC |
| | | CTGGATGAACTGCTCCGCCCTCTGTCTCTTTGGCA |
| | | GGGTTGGAGCCCACTGTGGCCTGAGCGACTTCTAT |
| | | GGCTCCCTTTTCTGTGATTTTCATGGTTTCTGAGC |
| | | TCTTTTCCCCCGCTTTATGATTTTCTCTTTTTGTC |
| | | TCTCTCTTGCTAAACCTCCTTCGTATATATGCCCT |
| | | CTCAGGTTTCATTTCTGAATCATCTACTGTGAACT |
| | | ATTCCCATTGTTTGCCAGAAGCCCCCTGGTTCTTC |
| | | CTTCTAGACACCAGGCAAGGGGCAGGAGGTAAGAG |
| | | GCAGGAGTCCATAAAACAGCCCTGAGAGCCTGCTG |
| | | GGTCAGTGCCTGCTGTCAGAA |
| 64 | CD36 promoter | CTGGCCTCTGACTTACTTGGATGGGAAATAGCCAA |
| | fragment | AAAAAAAAAAAATGCTGAATATCTCAGATATAGGT |
| | | AATGGGTCTTCACCAGAACATAAAAATAGACCCTT |
| | | ATTAGCCATATCAGTAATGTGCTGTGTGGGGGATT |
| | | TTTTTTTTCTTTCAATTCCTCTGGCAACAAACCAC |
| | | ACACTGGGATCTGACACTGTAGAGTGCTTTCTCTT |
| | | CTCTTTTTTTGGGGGGGGGAGGGGGTGTGGTTGCA |
| | | TATTTAAACTCTCACGCATTTATGTAATGAGGACT |
| | | GCAGTGTAGGACTTTCCTGCAGAATACCATTTGAT |
| | | CCTATTAAGAATTGTCCAAATGTTGGAGCATTTGA |
| | | TTGAAAAATCCTTCTTAGCCATTTTAAAGGTAAGT |
| | | TGTATGATTTTTCTTTAAATAAAAAAGATTAAGGG |
| | | ATTTTTCCCATCACCCAGATGACGGGAGGTGACAC |
| | | TACTGTATAAATACTCCTAAGAAGTTATATAGGAG |
| | | GACAGGAAAAAAG |
| 65 | CD36 promoter | CAGGTGTGAGCCACTGCACCCGGCCACTCCCAAAT |
| | fragment | GGTTCTGATGCCTTAAGTGGAACTACCATTGAGTC |
| | | AACACATATTCATATTATGCTGACTCTATCCCAGA |
| | | TACTGAAAAGGACACTAAGGATGCAAAGGTGATTC |
| | | CAACAGACATGGTACCTGTCTAGGGATTGTTCAGT |
| | | CTAGACACAAATGACAGCCATGGAATGCTCCAATT |
| | | AAAAGAAAAATATTTTGAGTAAAACTATTTAAAAC |
| | | ACTGGTAATCACATTATCAGGATTCATTCACGGCC |
| | | GGGCGCGGTGGTTCACGCCTGTAATCCCAGCACTT |
| | | TGGGAGGCCGAGGGGAGTGGATCACGAGGTCAGGA |
| | | GATGGAGACCATCCTGGCTAACACGGTGAAACCCC |
| | | GTCTCTACTAAAAATACAAAAAATTAGCCGGGCAT |
| | | GGTGGCGGGCACCTGTTGTCCCAGCTACTCGGGAG |
| | | TCTGAGGCAGGAGAATGGCGTGAACCTGGGAGGCG |
| | | GAGCTTGCAGTGAGCCGAGATCGCGCCACTGCACA |
| | | CCAGCCTGGATGGCAGAGCGAGACTCCGTTTCAAA |
| | | AAAAAAAAAGAAAGAAAAGATTCATTCACAGTAAA |
| | | CAAACAAACAAACAAACAAAAAACTCTTCGTTGAT |
| | | TTCAATCTGGAAATGAAAAAGAAAAGAAGAAAAGG |
| | | AAAGGAAAAAGAATTCTGACTCATTGATAGATTTG |
| | | TTTAGAAATGTTTTTCTTTTTTCTGTTAGAAAGCT |
| | | CAGAGTTAAAGAGTGGACAAGAGAAATAACATTTT |
| | | TTCAACTCTTCCATTTTCTAAATAAAACACTGCCA |
| | | CTATACATTGGAGTATGGGATATTATTTCCACTTT |
| | | AAAGATAAGGAAACTGACTCAAATACACGGAAAAC |
| | | AGGATTTAGTAAACGTGATTTATTTGGAACGTGAT |
| | | CTCAGATCCACAGAGGTGAGAAAAACAGGAAAGGA |
| | | GAAAGAAAAAGTGCTGATTGAGAGTGTGGTCTCAG |
| | | CTGGAGACGCTCTTAACTCT |
| 66 | LPL promoter | CTGCAGGAGTATTCTATATAAGATAGCAGGCAGTT |
| | fragment | CTGCACGTCTGTTTATATGCCATTCACACCATGAA |
| | | CACATCCAATGCAATTGTATTCATCTTCTGTCATT |
| | | TTTTAAAATTAGAGTTTACTACATGATTTTTGGAG |
| | | GACGGCCATATCATAGTTATTCATCCCTTGCATGG |
| | | TCTGTTCTCGCCACTTCTAAAGTTCTGCGAAGTTT |
| | | AATTTTTGGCAGGTTTCAAGATTCTCTACATACTT |
| | | TGTAGGTTAGAGTGAACGTGCACAGTATATTGTGT |
| | | AATAATTGCAATACAAGACGGTGTGTTGTGTTGTG |
| | | GCACGGAATGCATCAACTTAAAGATAGAATTGTTA |
| | | TGCAGAATAGAGACCTTTGAAATATTTGGGTATTA |
| | | TCAACTTTTATTTCATTTTTGAGGAAACTTAAAAG |
| | | TGAGCCCAGTTTTTAAAAAGCATTCATTGTTCTAA |
| | | CCTACAATTGTGCATTGTATGTTTGTGTGTGTGTG |
| | | ACGGAGGGTGGGATGAATAAAAGGGGACATTAAAA |
| | | ATATTCAGACCATAAATATAGTAGATTGGAGGTTC |
| | | TGATTTGATGAGCCAGTTTCTCAGCCATAAACTGA |
| | | GAGGGGGTTGGGGATACACTTCATTGTCCTTCCTG |
| | | GCTAATGTAAATCCCTTATATTTAAAAAGATATTT |
| | | AAAAGTATTCCAAGCATTTTGGCAGAAAAGCATAG |
| | | TATCTAATGTTATTTTTTTCTTATTTTATGTGCAT |
| | | GCCTCTTATCCATTTAAAAATAGCTTTACTGACCT |
| | | ATAATTTACACACTATATAATTCTCCCATTGAAAG |
| | | TGCATAATTCTGTTGCTTTTAGTATATTTACAGAG |
| | | TTGTGCAGCATCAGCATAATGTAATCTAGAACATT |
| | | GTCATCAACTACCCCCAAATCTCTATTCTTCCCTT |
| | | CCCCTATTAATTACCCAGCCCCAGGCAAGCACTGA |
| | | TCTACTTTTGGTCTCTATGGATTTGTCTATTTGTG |
| | | GACACTTTAAATGGAATCATACAATATGTGTCTTT |
| | | TGCGACTATCTTCTTTCACTTATCATAACTCAATA |
| | | CGGCTTTAGATTATTTGACCTCGATGTTCTGCCTC |
| | | TGAACATAAAATATTATCCTTGCATTCCTTGATGA |
| | | GTTTGAGGATTGAGAATAATTTGCATGAGACAAAA |
| | | ATTAGAAACTAGTTAGAGCAAGTAGGCTTTTCTCC |
| | | ATCACATAAGCTGATCCATCTTGCCAATGTTAAAA |
| | | CACCAGATTGTACAAGCACAAGCTGGGACGCAATG |
| | | TGTGTCCCTCTATCCCTACATTGACTTTGCGGGGG |
| | | TGGGGATGGGGTGCGGGGTGAGTGAGGGAGGACTG |
| | | CAAGTGACAAACAGGATTCGTCAAAAGAGAGGTGT |
| | | ATTAAAGTGCCGATCAAATGTAATTTAACAGCTAA |
| | | ACTTTCCCTCCTTGGAAAACAGGTGATTGTTGAGT |
| | | ATTTAACGTGAATCGATGTAAACCTGTGTTTGGTG |
| | | CTTAGACAGGGGGCCCCCGGGTAGAGTGGAACCCC |
| | | TTAAGCTAAGCGAACAGGAGCCTAACAAAGCAAAT |
| | | TTTTCCGTCTGCCCTTTCCCCCTCTTCTCGTTGGC |
| | | AGGGTTGATCCTCATTACTGTTTGCTCAAACGTTT |
| | | AGAAGTGAATTTAGGTCCCTCCCCCCAACTTATGA |
| | | TTTTATAGCCAATAGGTGATGAGGTTTATTTGCAT |
| | | ATTTCCAGTCACATAAGCAGCCTTGGCGTGAAAAC |
| | | AGTGTCAGACTCGATTCCCCCTCTTCCTCCTCCTC |
| | | AAGGGAAAGCTGCCCACTTCTAGCTGCCCTGCCAT |
| | | CCCCTTTAAAGGGCGACTTGCTCAGCGCCAAACCG |
| | | CGGCTCCAGCCCTCTCCAGCCTCCGGCTCAGCCGG |
| | | CTCATCAGTCGGTCCGCGCCTTGCAGCTCCTCCAG |
| | | AGGGACGCGCCCCGAGATGGAGAGCAAAGCC |
| 67 | LPL promoter | CCTCGATGTTCTGCCTCTGAACATAAAATATTATC |
| | fragment | CTTGCATTCCTTGATGAGTTTGAGGATTGAGAATA |
| | | ATTTGCATGAGACAAAAATTAGAAACTAGTTAGAG |
| | | CAAGTAGGCTTTTCTCCATCACATAAGCTGATCCA |
| | | TCTTGCCAATGTTAAAACACCAGATTGTACAAGCA |
| | | CAAGCTGGGACGCAATGTGTGTCCCTCTATCCCTA |
| | | CATTGACTTTGCGGGGGTGGGGATGGGGTGCGGGG |
| | | TGAGTGAGGGAGGACTGCAAGTGACAAACAGGATT |
| | | CGTCAAAAGAGAGGTGTATTAAAGTGCCGATCAAA |
| | | TGTAATTTAACAGCTAAACTTTCCCTCCTTGGAAA |
| | | ACAGGTGATTGTTGAGTATTTAACGTGAATCGATG |
| | | TAAACCTGTGTTTGGTGCTTAGACAGGGGGCCCCC |
| | | GGGTAGAGTGGAACCCCTTAAGCTAAGCGAACAGG |
| | | AGCCTAACAAAGCAAATTTTTCCGTCTGCCCTTTC |
| | | CCCCTCTTCTCGTTGGCAGGGTTGATCCTCATTAC |
| | | TGTTTGCTCAAACGTTTAGAAGTGAATTTAGGTCC |
| | | CTCCCCCCAACTTATGATTTTATAGCCAATAGGTG |
| | | ATGAGGTTTATTTGCATATTTCCAGTCACATAAGC |
| | | AGCCTTGGCGTGAAAACAGT |
| 68 | LEP promoter | GGTACCCAAGGGTGCGCGCGTGGCTCCTGGCGCGC |
| | fragment | CGAGGCCCTCCCTCGAGGCCCCGCGAGGTGCACAC |
| | | TGCGGGCCCAGGGCTAGCAGCCGCCCGGCACGTCG |
| | | CTACCCTGAGGGGCGGGGCGGGAGCTGGCGCTAGA |
| | | AATGCGCCGGGGCCTGCGGGGCAGTTGCGCAAGTT |
| | | GTGATCGGGCCGCTATAAGAGGGGCGGGCAGGCAT |
| | | GGAGCCCCGTAGGAATCGCAGCGCCAACGGTTGCA |
| | | AG |
| 69 | CIDEA promoter | CTTCAACAGTCCTGCAAGTCACAAGGCTTGTTGAC |
| | fragment | CACGGTGACCCTTTCTGAGAGCCAGCGGAGGAAGA |
| | | GGGCGGGCATAGGGTCAGGTCTTAAGAGATCAGGT |
| | | CAGTCTGCTCAAACCAGGTGGGCTCAGGAGGAGCC |
| | | GGCGAGGGTCCCGCTGCAGCTCCCGAAACGCTGTT |
| | | TCCAGGCGGCAGTCAGCAGTTGGGCACGAGAGGAT |
| | | ATCCTAGCAAGCCTCCATCCCAGAAAGTTACACCT |
| | | CTTACTGCCCGAGTCCCACCCCTCAACACTGAGCC |
| | | CACCTCTTCAGTCCTGTAGTGAGGGGTTCTGGGAA |
| | | CCCCTTGTAACTGCCCGCCTTCCTCGGAGGTCTCA |
| | | GAGGCCCTAATTGCGTCTTCCGATGGGAACAACCA |
| | | TGAGAGCAGGGTGTGGACGGAAGTCCCCCAGCCCC |
| | | AGCGGGGTTCACTCTTTGCCTCCTCACTCTGCGAG |
| | | GGTCCCTTACTACAGCTAAAAGGCAATTCATGCAG |
| | | TCCAAAGGCTCCTTTCCCCTCCCGTTCTGCATGCC |
| | | CTTCTCACTGTCCCCAAATGCTCATGACATGGCCT |
| | | TGAGAGCAGCGCAGCGGGCAGGAAGCCCAGGCGCC |
| | | TCGGAGAACCTGAAGGGCTGCAGGTCCCGGTGTCT |
| | | GCGGCAGCTTCCGCATCCCTGCAGGGAACCAGCGG |
| | | CTCGCTGAGCCTGGGGCTGGGCTCTGGGGGACTCC |
| | | AGAGCTGGAGGCAAGTGGCGCTCACATCCGTCTCC |
| | | TCACCTGCCCCACCCGTGGCCTGGGTTTAAAATCC |
| | | ACATACCCGTCTTTCCGCGGCCAAAGTGATGCTGC |
| | | CAGGATTGGTTATGACCCCAACTGCCCCGACCCCC |
| | | AGAAGTGCAAAACGAACAGGCTGGCAAGTGACCAA |
| | | AAGAGACCCGGGGAGCATCTGGGCTTCCAAGGTCC |
| | | TCGGTACGGCCCAAGGCAGCGAAGGACGCGCGGCT |
| | | CCAGGCTGCGGGAGCCAGGACGACCGGGGGCTCCC |
| | | AGAGCGCGAAGTCGCGATCCTCGGCGGTGGAGAGC |
| | | TCGTGCCAAAACGTCCTCCCCTGCGCCAGTCAGGC |
| | | CTTCGCGGGGCTGGCAGGCGGGCGGGGGCGGGGCC |
| | | GCCGCACTTTAAGAGGCTGTGCAGGCAGACAGACC |
| | | TCCAGGCCCGC |
| 70 | LIPE promoter | GGGGGCGTGCATATCTTAGCTGGGGAGAAGGCAAC |
| | fragment | TGGGGAGTACCTCTTGGCAAGTGACAAGCTGAGGC |
| | CIDEC promoter | CCAGACAAGGAATCTGTGGAATGACACGGGTGAAT |
| | | GACATGGGTGAGGAATCTCTCTCTCTGAGCCTCTG |
| | | TTTCCCTGTGTGAGGTGGGATGCCAGTCACAGCAG |
| | | GGTGGGCTGAGAGGGGTAAGTGGCAGGTCCAGGTT |
| | | ACCCAGTGTGGTCACACCACCCAGTAGGACCGACA |
| | | GGCAGCTCAGGCCCCTAGCTGAGCCTGGCACTGCC |
| | | CTTAATCGGCTTTTGTCCTTCCAGGAGGGACAGGC |
| | | AGGGCTGGCTACCTCCTCTCTCCCCCCAACAGCTG |
| | | GGGCTGGCTGTGCCAGGGCCAGACTAGGAGGCGGG |
| | | AGCTGAGATCACTGGGCCTTTGGCTCCCTGAGTCC |
| | | TCTCTGGGGACAGAGAGGGCAGCCTGGCTGGGTGA |
| | | GAGGGGACGGTTTGTTCAGGACTGGGGCTAGGACT |
| | | CCTGGGTCCTGAAGGATGAAGGGGCTGGGGGCCTG |
| | | GACTCCTGGGTCTGAGGGAGGAGGGGCTATGGGTG |
| | | TGGACTCCTGGGTTCTAAGGCAGAGGAGCCTGTAA |
| | | CAGGACTACCAGTTGAGCTGAGTCTGGGAGCAGGT |
| | | GGGGGGCAGAGCAGGGAGCTGAGCCCTCTACTCTG |
| | | TTTACAGCACGTGGTCCTCACTGATCTTTCTGGGT |
| | | GGGAGGTGGCTTGTGCGGCTACACCCTGGGCAGGC |
| | | CAGCCCCGCCCCCGGGTTTATTGCCCCAGGCTGCT |
| | | ACTGGCAC |
| 71 | fragment | TAATCCTCACCACTACCCTATAAAACAAGAATCAT |
| | (1,800/+21) | TATCGTCATTACACAGAGAAGAGAAACGGGCTCAC |
| | | AGAAGTCAAGTGATCTGTCTAAGCTCAAGCAAGCT |
| | | GATATGGGGTGGAGTTGGGACCACAACCCACATCT |
| | | GCCTGCTTTCTGTGCCCTCTGTGCTTGATCTCATG |
| | | TCATGGGCCATCCATTTTGCCAAAGCTTTCGAAAC |
| | | ATGCCTTCTCCCCTCCTTTTAGGCCTTTTCTGGAA |
| | | GTCAGGCTGGCTTGCAGCTCTCACCCCAAGCTCTT |
| | | TTCCCCAGCTGGGGCTGAAGGCTGGGGAGGAGCTT |
| | | CATGGGGGCTGGAAATAAATGAAGGGAGGGCTGGG |
| | | CGGGACAGGGCAGTATTTCCAGGAGGCTGTGAGGG |
| | | GGAGGTAAGGAGTTTGTTATTGTTTTGGGTTTGGG |
| | | GATTTTTTTTTCTTTGCTCCTGGAGAGCAGGTTAG |
| | | GTAGCTCTTGGCAGAGTTTTGCTAGGGATACGAAG |
| | | TGGGTGAGGCTGAGGCAAGCAGAATCCTCCTGCCT |
| | | CAGCCTCCCACGTAGCTAGGACTAGAGCTTCTGCA |
| | | GTGCTAAGTGGGAAAGGGGCTTTCTGCCTCTGTGG |
| | | GGCCTGGTCTGGAAAGTCCCCCAAATTGTTAAAAG |
| | | CAAAGAGTGATTGTCCCCAGCTGGCAAGGGGACTT |
| | | TTGTTTTATGTCCACCCCTTGTGCCCCTTGCTCTG |
| | | CGAAGCCCTACCTGTGCCGGGAGCTTCGAATAGGA |
| | | AGAGGACCTCAGGGCCAGGCATCCAGGGACTGAGG |
| | | GGCTACAGCAGAAACAGACCCAGAGGCACCCTTGG |
| | | CTAGGAAGAGCAGGATGAAGGTGTAATAATTTTGG |
| | | GAGTGGTCTAAGAAGACTTTCCTTTTAAAGAGTGG |
| | | AAAGCAATGAAACTGAGGCTTCAAGGCTGCAAAAA |
| | | TTTCGTCCTGGATCAGAGAAGGGCACTACAGGTGG |
| | | AGGGAACTATCTGTGCAAAGGGTTGGGCCCTTCAA |
| | | GAGTTGGAGCACAGAGGTTTAGGAGAAAAAGTGGT |
| | | GACAGATGAGCCCAGGGCTATCTGCTTAGACCTGT |
| | | GATCTAATATGGGTGCCAACAGCCACATGGCAATG |
| | | TGGCTATTTAAATTTAAATTAGTTAAAATTCAATA |
| | | AAGTAAAAAATACAGCTCCTGTGCCAGGTGCAGTG |
| | | GCTCACGCCTGTAATCCCCGCACTTTGGGAGGCCA |
| | | AGACAGGGAGATCACCTGAGGTCAGGAGTTCAAGA |
| | | CCAGCCTAGCCAACATGGTGAAACCCAGTCTCTAC |
| | | TAAAAATCCAAACAAAACGAAACTAAACTAAAAAT |
| | | TAGCTGGGCGTGGTGGCAGGTGCCTGTAATCCCAG |
| | | CTACGGGGGAGGCTGAGGCAGGATAATTGCTTGAA |
| | | CCCAGGAGGCGGAGGTTGTAGTGAGCAAGATCGCC |
| | | CCATTGCACTCCAGCCTGGGTGACAAGAGCGAAAC |
| | | TCAAAAAAAAAAAAAATTCAGCTCCTGAATCACAC |
| | | TAGTTATGTTTCAAATACTCAATAGCCACATGGGG |
| | | CTAGTAGCAATGGTATTGGAGAGTATGTACAAGGC |
| | | ATGCCTCTATCATCACAGAATGTTCTTTTGGCCAC |
| | | TGTGAAGCCTCAGGAAGGGGCTCGGATTGCTCAAG |
| | | GACCCATGGGAGAGAGGAGGCTTTGACTGGGCTGC |
| | | CTGCCTGTGAGGTCTCTGGACTAGAGGTGAGGGAA |
| | | CGGTCCCCAGAAGTGTGAGGTGGTGTGACTCACCT |
| | | CCTGCCAGCGTAAAGTGGACTCTGTCCCCATGGAC |
| | | AGGAACAAATCGGGCAAGGCTTCTTCAGCCCTGAG |
| | | TTGGGTTATCTGCTCAGTGTGACCAGAGCCAGGGG |
| | | AT |
| 72 | TUSC5 promoter | ACAGGCACCCACCACCACGCTAATTTTTGTATTTT |
| | fragment | TAGTAGAGACAGGGTTTTGCCATGATGGCCAGGCT |
| | | GGTCTCGAACTGCTGACCTCAAGTGATCCACCTGC |
| | | CTCAGCCTCCCAAAGTGCTGGGATTACAGGCATGA |
| | | GTCACCGTGCCCGGCTGAGTTTTCAAATCTTATTC |
| | | ATTTCCAGGTGTCCTTTCAGCCCCATATCTCCCAC |
| | | TGAGCTGTGCGGCAGGATCTCGGCTCACTGCAGCC |
| | | TGGACCTCCCAGGCTCAAGCGATTCTCCTCCCTCA |
| | | GCCTCCCAAGTAGCTGGGATTACAGGCATGTACCA |
| | | CCACGCCCATATATTTTTTGTATTTTTTGTAGAGA |
| | | TGGGATTTTGCCATGTTGCCCAGGCTGGTGTCAAA |
| | | CTCCTGGGCTAAAGCAGTCCTTCTGCCTCGGCCTC |
| | | CCGAAGTGCTGGGTTTATGGGCATGAGCCATGGCA |
| | | TCCCGCCTGATCGTAGGATTTTAAACTATTTCATA |
| | | CAACCACTTTCAAACCACACACACACATCCGTGGC |
| | | GGCTCCAGATCACACTTGTAAAACAATGTAAGGAG |
| | | GGGCGTCAGGAATATAGAAAGAGATGCCGAGGATA |
| | | GAGAAGAAGGCAGAGACAGAGGGAGAAAACCTAAG |
| | | AGAGAACAAAGGAGGAGAATCCGATAGGACAAACA |
| | | GCCTGAGGCCCTTCCGAGCTGGCATCTGCCCGGAG |
| | | CAGCTGACCTAGGGGCTCTCTGCCACCGGCTCTTT |
| | | AGCACCCCAGAATCGTTCTGATGGGCATGAGGTGG |
| | | ACCCAACACTCCCCCTTATCCTCTTCCCCACCTGG |
| | | TGGTGAGCTACGTAGAAACCTACTCATGGGGCCCT |
| | | GCTGGTCCATGCAGGGTCCAGGCAGGATCCAGGGG |
| | | CTACCCTGGTGCTCACATCTCCCCTTCCCTTCTGG |
| | | AGTCGGGAGCCAGGCTTTGGCCTGGATGCTCCCAG |
| | | CGGTTGGGGGTCAGGGTCTGGGTGGGATGTAATAG |
| | | GGGGCCCTAAGGTCCAGCATTTCCCAACAGAAAGA |
| | | AAGAAGTACACGCTTGCTTTGGAAGAAGCAGCGGA |
| | | GGGCTCAGTTCCCAGGACAGCTGAAGACACATCCC |
| | | TGTGGGGCCCAAAGTGTCTTTCTCCCCATCCAGAG |
| | | ACCCTGCCCAGAGGAGCAGGCCCTGGAGCTGCAAC |
| | | AGAAACCGGAGGCTGGCCCAAGTCCCTCTGGGCTG |
| | | GGCTGGGCTGGGCTGGGCAGAGCTGGGCTGGGCTG |
| | | GGCTGGGCTGGGGCAGAGGCAAAATCCTGCCAGGG |
| | | TGGTTCTCTGGGGAGAGGGGAATTGAAAAGGAAAA |
| | | GCATCACCAAGGAGGGGGAGAGGGAGGGAGGCCGT |
| | | GTGCGCACACGCACACACACACACACACACACACA |
| | | CACACACACACAGCCTCCCTGACTGAGCCTGAGCC |
| | | TCCCTGGGAAGAGGCGCATTCTTCTCTCTGCTCTC |
| | | TGAGCTTTCCGTTGCTTCCCTGCCCATCTGTGCTC |
| | | TCAGCAGCACCAGCAAAGTTGGCCTCAAACTTGAA |
| | | CAAAGCTGCAGGCTCCAGCGTCTTTCTGGGAGCTC |
| | | CGCGGGGGCAGCAGGCAGGCCCCAGCCTCTGAACT |
| | | CAGCTGGCTTGAGAAGCTCAGCCCAACCCTTCCAG |
| | | CACCCAGCCGGCCCTCCGTCTCCTGAGGGACGCCC |
| | | CTGCCCCCGACCTGGAGGCCCTCAGTCTGGGCTGG |
| | | GGAATGGCGCGC |

A transcriptional promoter disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to any one of SEQ ID NOs: 1 and 56-72.

A transcriptional promoter disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 100 consecutive nucleotides of any one of SEQ ID NOs: 1 and 56-72.

A transcriptional promoter disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 250 consecutive nucleotides of any one of SEQ ID NOs: 1 and 56-72.

A transcriptional promoter disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 500 consecutive nucleotides of any one of SEQ ID NOs: 1 and 56-72.

A transcriptional promoter disclosed herein can comprise, consist essentially of, or consist of a nucleotide sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity to any one of SEQ ID NOs: 1 and 56-72.

In some embodiments, a transcriptional promoter comprises, consists essentially of, or consists of a nucleotide sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity to any one of SEQ ID NOs: 1 and 56-72.

In some embodiments, the transcriptional promoter comprises, consists essentially of, or consists of the nucleotide sequence of any one of SEQ ID NOs: 1 and 56-72.

In some embodiments, the transcriptional promoter comprises a nucleotide sequence with one or more insertions, deletions, and/or substitutions relative to any one of SEQ ID NOs: 1 and 56-72.

For example, the transcriptional promoter can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide insertions relative to any one of SEQ ID NOs: 1 and 56-72.

In some embodiments, the transcriptional promoter comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide insertions relative to any one of SEQ ID NOs: 1 and 56-72.

In some embodiments, the transcriptional promoter comprises 1, 2, 3, 4, 5, 6, 7, 8, 9, 10, 11, 12, 13, 14, 15, 16, 17, 18, 19, 20, 25, 30, 35, 40, 45, or 50 nucleotide insertions relative to any one of SEQ ID NOs: 1 and 56-72.

The one or more insertions can be at the 5′ end, the 3′ end, within the nucleotide sequence, or a combination thereof. The one or more insertions can be contiguous, non-contiguous, or a combination thereof.

In some embodiments, the transcriptional promoter comprises a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide deletions relative to any one of SEQ ID NOs: 1 and 56-72.

In some embodiments, the transcriptional promoter comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide deletions relative to any one of SEQ ID NOs: 1 and 56-72.

In some embodiments, the transcriptional promoter comprises 1, 2, 3, 4, 5, 6, 7, 8, 9, 10, 11, 12, 13, 14, 15, 16, 17, 18, 19, 20, 25, 30, 35, 40, 45, or 50 nucleotide deletions relative to any one of SEQ ID NOs: 1 and 56-72.

The one or more deletions can be at the 5′ end, the 3′ end, within the nucleotide sequence, or a combination thereof. The one or more deletions can be contiguous, non-contiguous, or a combination thereof.

In some embodiments, the transcriptional promoter comprises a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide substitutions relative to any one of SEQ ID NOs: 1 and 56-72.

In some embodiments, the transcriptional promoter comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide substitutions relative to any one of SEQ ID NOs: 1 and 56-72.

In some embodiments, the transcriptional promoter comprises 1, 2, 3, 4, 5, 6, 7, 8, 9, 10, 11, 12, 13, 14, 15, 16, 17, 18, 19, 20, 25, 30, 35, 40, 45, or 50 nucleotide substitutions relative to any one of SEQ ID NOs: 1 and 56-72.

The one or more substitutions can be at the 5′ end, the 3′ end, within the nucleotide sequence, or a combination thereof. The one or more substitutions can be contiguous, non-contiguous, or a combination thereof.

A transcriptional promoter (or promoter) can describe a region of DNA involved in initiating or increasing transcription of a particular gene, such as a transgene disclosed herein. A promoter can be located near a transcription start sites of a gene, on the same strand and upstream on the coding sequence. A Promoter can be, for example, about 100-1000 base pairs long. Promoters can contain specific DNA sequences and response elements that provide an initial binding site for RNA polymerase and for proteins called transcription factors that recruit RNA polymerase. A promoter can be a part of an expression regulatory system that comprises other regulatory regions, e.g., enhancers, silencers, and/or boundary elements/insulators to direct the level of transcription of a given gene. An enhancer can be a regulatory element that is distant from the transcriptional start site. In some embodiments, an enhancer that is natively distant from a transcriptional start site is in relative proximity to the transcriptional start site in a polynucleotide construct disclosed herein.

A transcriptional promoter can comprise a transcription factor binding site. A transcriptional promoter can comprise two or more transcription factor binding sites.

A transcriptional promoter can be, comprise, consist essentially of, or consist of a core promoter, for example, the minimal portion of a promoter that is required to initiate transcription. A core promoter can comprise, for example, (1) a transcription start site (TSS), (2) an RNA polymerase binding site (e.g., an RNA polymerase II binding site in a promoter for a gene encoding a messenger RNA), (3) a general transcription factor binding site (e.g., a TATA box having a consensus sequence TATAAA, which can be a binding site for a TATA-binding protein (TBP)), (4) a B recognition element (BRE), (5) a proximal promoter (e.g., of approximately 250 bp) that contains regulatory elements, (6) transcription factor binding sites (e.g., an E-box having the sequence CACGTF, which is a binding site for basic helix-loop-helix (bHLH) transcription factors including BMAL11-Clock and cMyc), and/or (7) a distal promoter containing additional regulatory elements.

A transcriptional promoter disclosed herein can be, for example, (1) an AT-based class promoter, (2) a CG-based class promoter, (3) an ATCG-compact class promoter, (4) an ATCG-balanced class promoter, (5) an ATCG-middle class promoter, (6) an ATCG-less class promoter, (7) an AT-less class promoter, (8) a CG-spike class promoter, (9) a CG-less class promoter, or (10) an ATspike class promoter.

A transcriptional promoter disclosed herein can be a unidirectional promoter. A transcriptional promoter disclosed herein can be bidirectional promoter.

A transcription factor can be a sequence-specific DNA-binding factor that binds to specific sequence(s) within a transcriptional promoter, thereby regulating the transcription of a gene (e.g., transgene) in operable proximity to and downstream of the promoter. Transcription factors can include can activators, which promote transcription, and repressors, which block or negatively regulate transcription by reducing the recruitment or binding of an RNA polymerase. Transcription factors can contain (1) one or more DNA-binding domains (DBDs), which facilitate sequence-specific binding to a cognate transcription factor binding site (e.g., response element) within a transcriptional promoter; (2) one or more signal-sensing domains (SSDs), which can include ligand binding domains that are responsive to external signals; and/or (3) one or more transactivation domains (TADs), which contain binding sites for other proteins, including transcription coregulators.

Transcription factors can be categorized according to structural features of the DNA-binding domain. A transcription factor can comprise a basic helix-loop-helix domain, basic-leucine zipper (bZIP domain), C-terminal effector domain of a bipartite response regulator, GCC box domain, helix-turn-helix domain, homeodomain, lambda repressor-like domain, serum response factor-like (srf-like) domain, paired box domain, winged helix domain, zinc finger domain, multi-Cys2His2 zinc finger domain, Zn2Cys6 domain, and/or Zn2Cys8 nuclear receptor zinc finger domains.

In some embodiments, a promoter disclosed herein improves selectivity of expression of a polynucleotide construct disclosed herein compared to an mRNA payload, which can be translated by any cell it is delivered to.

In some embodiments, a promoter disclosed herein is an inducible promoter. In some embodiments, a promoter disclosed herein is not an inducible promoter. In some embodiments, a promoter disclosed herein is a constitutive promoter (e.g., eliciting substantively constitutive expression in adipocytes or white adipocytes). In some embodiments, a promoter disclosed herein is not a constitutive promoter.

An expression regulatory region disclosed herein can comprise any suitable number of promoters. An expression regulatory region can comprise at least 1, at least 2, at least 3, at least 4, or at least 5 promoters. An expression regulatory region can contain at most 1, at most 2, at most 3, at most 4, or at most 5 promoters. An expression regulatory region can comprise 1, 2, 3, 4, or 5 promoters.

### B. Cytotoxic Protein

A polynucleotide construct can comprise a transgene that encodes a therapeutic protein, such as a cytotoxic protein. Expression of the transgene can be driven by a transcriptional promoter disclosed herein. The transgene can be operatively linked to a transcriptional promoter disclosed herein. The transgene can be under regulatory control of a transcriptional promoter disclosed herein.

A cytotoxic protein can reduce, prevent, and/or substantially eliminate the growth or survival of a cell that expresses it, for example, an adipocyte, such as a white adipocyte.

In some embodiments, a cytotoxic protein induces a non-inflammatory form of cell death. In some embodiments, a cytotoxic protein induces a programmed form of cell death. In some embodiments, a cytotoxic protein induces death of a cell by apoptosis. In some embodiments, a cytotoxic protein induces an inflammatory form of cell death.

A cytotoxic protein can be, comprise, consist essentially of, or consist of a caspase or a catalytic domain thereof. A cytotoxic protein can be a caspase, for example, caspase 1, caspase 3, caspase 8, or caspase 9. A cytotoxic protein can comprise a catalytic domain of a caspase, for example, a catalytic domain of caspase 1, caspase 3, caspase 8, or caspase 9. Proteins of the caspase family can execute the genetic program that leads to cell death.

A cytotoxic protein can be, comprise, consist essentially of, or consist of caspase 1, or a catalytic domain thereof. A cytotoxic protein can be, comprise, consist essentially of, or consist of caspase 3, or a catalytic domain thereof. A cytotoxic protein can be, comprise, consist essentially of, or consist of caspase 8, or a catalytic domain thereof. A cytotoxic protein can be, comprise, consist essentially of, or consist of caspase 9, or a catalytic domain thereof.

A cytotoxic protein can be, comprise, consist essentially of, or consist of a non-inducible caspase, such as a non-inducible caspase 1, caspase 3, caspase 8, or caspase 9, or a non-inducible protein that comprises a catalytic domain of caspase 1, caspase 3, caspase 8, or caspase 9.

A cytotoxic protein can be, comprise, consist essentially of, or consist of a self-activating caspase, such as a self-activating caspase 1, self-activating caspase 3, self-activating caspase 8, or self-activating caspase 9, or a self-activating protein that comprises a catalytic domain of caspase 1, caspase 3, caspase 8, or caspase 9. A self-activating caspases can activate in the absence of an inducing agent, for example, a chemical inducer of dimerization (CID), such as rapamycin. Self-activating caspases can be advantageously employed, for example, for the induction of apoptosis in a rapidly dividing cell, where an inducible caspase protein would be diluted out before administration of an inducing agent.

A cytotoxic protein can be, comprise, consist essentially of, or consist of an inducible caspase, such as an inducible caspase 1, inducible caspase 3, inducible caspase 8, or inducible caspase 9, or an inducible protein that comprises a catalytic domain of caspase 1, caspase 3, caspase 8, or caspase 9.

An inducible cytotoxic protein, such as an inducible caspase disclosed herein, can be in an inactive state until contacting with a chemical or biological compound that activates the cytotoxic protein. An inducible cytotoxic protein can provide an additional layer of regulation of the activity of the cytotoxic protein over, for example, the promoter that is preferentially active in an adipocyte or white adipocyte. For example, to induce apoptosis of an adipocyte, contacting the adipocyte with an inducing agent (e.g., rapamycin or a structural analogue thereof) and/or administering the inducing agent to a subject can be required.

An inducible cytotoxic protein, such as an inducible caspase disclosed herein, can be activated by contacting with a macrolide. An inducible cytotoxic protein, such as an inducible caspase disclosed herein, can be activated by contacting with rapamycin or a structural analogue thereof. An inducible cytotoxic protein, such as an inducible caspase disclosed herein, can be activated by contacting with another inducing agent, such as AP20187.

An inducible cytotoxic protein can comprise caspase 9 fused to a human FK506 binding protein (FKBP) to allow conditional dimerization using the small molecule AP20187 (which can be a synthetic analog of FK506).

An inducible cytotoxic protein can be a rapamycin-inducible cytotoxic protein. For example, a cytotoxic protein can comprise, consist essentially of, or consist of a rapamycin-inducible caspase, such as a rapamycin-inducible caspase 1, rapamycin-inducible caspase 3, rapamycin-inducible caspase 8, or rapamycin-inducible caspase 9, or a rapamycin-inducible protein that comprises a catalytic domain of caspase 1, caspase 3, caspase 8, or caspase 9.

A rapamycin-inducible cytotoxic protein can utilize a double-rapamycin inducible system for Caspase 3 and 9 that employs RU486 and chemical inducers of dimerization (CID).

A rapamycin-inducible cytotoxic protein can utilize rapamycin inducible caspase 8 system by employing the ARIAD™ homodimerization system (FKC8; ARIAD Pharmaceuticals).

A rapamycin-inducible cytotoxic protein can comprise a full length rapamycin-inducible caspase 9. For example, a rapamycin-inducible cytotoxic protein can comprise a caspase recruitment domain (CARD; GenBank NM001 229) linked to two 12 kDa human FK506 binding proteins. The FK506 binding proteins can be, for example, FKBP12 (GenBank AH002 818) that optionally contain an F36V mutation. A linker (e.g., a Ser-Gly-Gly-Gly-Ser linker, or another linker disclosed herein) can connect the FK506 binding proteins and/or the FKBPs and caspase 9.

A rapamycin-inducible cytotoxic protein can include a dimerization domain, such as an FKBP, FK506, and/or FRB binding protein domain, that binds to rapamycin or a structural analog thereof. Illustrative genes (e.g., human genes) encoding FKBP domains include AIP, AIPL1, FKBP1A, FKBP1B, FKBP2, FKBP3, FHBP5, FKBP6, FKBP7, FKBP8, FKBP8, FKBP9L, FKBP10, FKBP11, FKBP14, FKBP15, FKBP52, and LOC541473.

Rapamycin and rapamycin analogues can induce dimerization (e.g., heterodimerization) by generating an interface between the FRB domain of mTOR and FKBP12. This association can result in FKBP12 blocking access to the mTOR active site, thereby inhibiting its function. While mTOR is a very large protein, the precise small segment of mTOR required for interaction with Rapamycin is known and can be used in a rapamycin-inducible cytotoxic protein to facilitate dimerization and activation. Dimerization mediated by rapamycin or a structural analogue thereof can be employed to induce dimerization of a rapamycin-inducible cytotoxic protein (e.g., caspase), including multi-domain rapaCaspase proteins. The dimerization can be, for example, heterodimerization (e.g., of distinct polypeptide chains or domains in the same polypeptide chain), or homodimerization (e.g., of distinct polypeptide chains or domains in the same polypeptide chain).

Rapamycin-inducible cytotoxic proteins, including rapamycin-inducible caspases, can include (i) an FRB domain (e.g., from, based on, or derived from mTOR); (ii) an FKBP12 domain; and (iii) a caspase or functional fragment thereof. Heterodimerization between an FRB domain of a first rapaCaspase fusion protein and an FKB12 domain of a second rapaCaspase fusion protein can activate the caspase activity. In some embodiments, a first heterodimerization domain of a rapamycin-inducible cytotoxic protein disclosed herein comprises an FK506-binding protein (FKBP) and a second heterodimerization domain comprises an FRB domain (e.g., that is from, based on, or derived from mTOR). The rapamycin inducible cytotoxic protein can comprise one polypeptide chain (e.g., with domains that heterodimerize), or two polypeptide chains that dimerize.

The rapamycin or structural analog thereof can bind with a high affinity to the FKBP12 protein, creating a drug-protein complex that subsequently binds to a second protein or domain, such as FKBP-rapamycin binding (FRB) domain or a derivative thereof. The FRB domain of mTOR can comprise an approximately 89 amino acid polypeptide.

A rapamycin-inducible cytotoxic protein can be activated by rapamycin. A rapamycin-inducible cytotoxic protein can be activated by a structural analogue of rapamycin, such as FK506, C-20-methyllyrlrapamycin (MaRap), C16(S)-Butylsulfonamidorapamycin (C16-BS-Rap), C16-(S)-7-methylindolerapamycin (AP21976/C16-AiRap), C16-(S)-3-mehylindolerapamycin (C16-iRap), Sirolimus, Tacrolimus, Everolimus, Temsirolimus, or Deforolimus. A structural analog of rapamycin can be functionalized at C16 and/or C20 of rapamycin. Contacting a cell expressing a rapamycin-inducible cytotoxic protein with rapamycin or a structural analogue thereof can facilitate the dimerization of the rapaCasp9 protein, which in some embodiments triggers apoptosis in a target cell, such as an adipocyte.

TABLE 2 provides non-limiting examples of cytotoxic proteins and domains thereof disclosed herein. FKBP12 can describe the amino acid sequence of FKBP12. dCasp9 can describe the catalytic domain of Casp9. L1 can describe a one repeat linker. FMD-2A can describe a Foot and mouth disease 2A like peptide ERAV. Optionally, another 2A peptide disclosed herein can be used, e.g., instead. FRB can describe the FRB domain of mTOR. L3 can describe a two-repeat linker. FRBw can describe a codon wobbled FRB. Optionally, another linker disclosed herein or a repeat thereof can be used instead of a linker sequence in any of the sequences in TABLE 2.

TABLE 2

| SEQ | | |
|---|---|---|
| ID | | |
| NO | Description | Sequence |
| 3 | FRB-FKBP12- | MASRILWHEMWHEGLEEASRLYFGERNVKG |
| | L3-dCasp9 | MFEVLEPLHAMMERGPQTLKETSFNQAYGR |
| | | DLMEAQEWCRKYMKSGNVKDLLQAWDLYYH |
| | | VFRRISKLEYSGGGSLEGVQVETISPGDGR |
| | | TFPKRGQTCVVHYTGMLEDGKKFDSSRDRN |
| | | KPFKFMLGKQEVIRGWEEGVAQMSVGQRAK |
| | | LTISPDYAYGATGHPGIIPPHATLVFDVEL |
| | | LKLESGGGGSGGGGSGGGGSGVDGFGDVGA |
| | | LESLRGNADLAYILSMEPCGHCLIINNVNF |
| | | CRESGLRTRTGSNIDCEKLRRRFSSLHFMV |
| | | EVKGDLTAKKMVLALLELAQQDHGALDCCV |
| | | VVILSHGCQASHLQFPGAVYGTDGCPVSVE |
| | | KIVNIFNGTSCPSLGGKPKLFFIQACGGEQ |
| | | KDHGFEVASTSPEDESPGSNPEPDATPFQE |
| | | GLRTFDQLDAISSLPTPSDIFVSYSTFPGF |
| | | VSWRDPKSGSWYVETLDDIFEQWAHSEDLQ |
| | | SLLLRVANAVSVKGIYKQMPGCFNFLRKKL |
| | | FFKTSAS |
| 4 | FKBP12- | MLEGVQVETISPGDGRTFPKRGQTCVVHYT |
| | dCasp9 | GMLEDGKKFDSSRDRNKPFKFMLGKQEVIR |
| | | GWEEGVAQMSVGQRAKLTISPDYAYGATGH |
| | | PGIIPPHATLVFDVELLKLESGGGSGVDGF |
| | | GDVGALESLRGNADLAYILSMEPCGHCLII |
| | | NNVNFCRESGLRTRTGSNIDCEKLRRRFSS |
| | | LHFMVEVKGDLTAKKMVLALLELAQQDHGA |
| | | LDCCVVVILSHGCQASHLQFPGAVYGTDGC |
| | | PVSVEKIVNIFNGTSCPSLGGKPKLFFIQA |
| | | CGGEQKDHGFEVASTSPEDESPGSNPEPDT |
| | | PFQEGLRTFDQLDAISSLPTPSDIFVSYST |
| | | FPGFVSWRDPKSGSWYVETLDDIFEQWAHS |
| | | EDLQSLLLRVANAVSVKGIYKQMPGCFNFL |
| | | RKKLFFKTSAS |
| 5 | FRB-dCasp9 | MASRILWHEMWHEGLEEASRLYFGERNVKG |
| | | MFEVLEPLHAMMERGPQTLKETSFNQAYGR |
| | | DLMEAQEWCRKYMKSGNVKDLLQAWDLYYH |
| | | VFRRISKLEYSGGGSGVDGFGDVGALESLR |
| | | GNADLAYILSMEPCGHCLIINNVNFCRESG |
| | | LRTRTGSNIDCEKLRRRFSSLHFMVEVKGD |
| | | LTAKKMVLALLELAQQDHGALDCCVVVILS |
| | | HGCQASHLQFPGAVYGTDGCPVSVEKIVNI |
| | | FNGTSCPSLGGKPKLFFIQACGGEQKDHGF |
| | | EVASTSPEDESPGSNPEPDATPFQEGLRTF |
| | | DQLDAISSLPTPSDIFVSYSTFPGFVSWRD |
| | | PKSGSWYVETLDDIFEQWAHSEDLQSLLLR |
| | | VANAVSVKGIYKQMPGCFNFLRKKLFFKTS |
| | | AS |
| 6 | FKBP12- | MLEGVQVETISPGDGRTFPKRGQTCVVHYT |
| | dCasp9-2A- | GMLEDGKKFDSSRDRNKPFKFMLGKQEVIR |
| | FRB-FRBw | GWEEGVAQMSVGQRAKLTISPDYAYGATGH |
| | | PGIIPPHATLVFDVELLKLESGGGSGVDGF |
| | | GDVGALESLRGNADLAYILSMEPCGHCLII |
| | | NNVNFCRESGLRTRTGSNIDCEKLRRRFSS |
| | | LHFMVEVKGDLTAKKMVLALLELAQQDHGA |
| | | LDCCVVVILSHGCQASHLQFPGAVYGTDGP |
| | | VSVEKIVNIFNGTSCPSLGGKPKLFFIQAC |
| | | GGEQKDHGFEVASTSPEDESPGSNPEPDAT |
| | | PFQEGLRTFDQLDAISSLPTPSDIFVSYST |
| | | FPGFVSWRDPKSGSWYVETLDDIFEQWAHS |
| | | EDLQSLLLRVANAVSVKGIYKQMPGCFNFL |
| | | RKKLFFKTSASQCTNYALLKLAGDVESNPG |
| | | PGVQVETISPGDGRTFPKRGQTCVVHYTGM |
| | | LEDGKKVDSSRDRNKPFKFMLGKQEVIRGW |
| | | EEGVAQMSVGQRAKLTISPDYAYGATGHPG |
| | | IIPPHATLVFDVELLKLESGGGGSGGGGSM |
| | | LEGVQVETISPGDGRTFPKRGQTCVVHYTG |
| | | MLEDGKKFDSSRDRNKPFKFMLGKQEVIRG |
| | | WEEGVAQMSVGQRAKLTISPDYAYGATGHP |
| | | GIIPPHATL VFDVELLKLES |
| 7 | Full-length | MDEADRRLLRRCRLRLVEELQVDQLWDALL |
| | caspase 9 | SSELFRPHMIEDIQRAGSGSRRDQARQLII |
| | | DLETRGSQALPLFISCLEDTGQDMLASFLR |
| | | TNRQAAKLSKPTLENLTPVVLRPEIRKPEV |
| | | LRPETPRPVDIGSGGFGDVGALESLRGNAD |
| | | LAYILSMEPCGHCLIINNVNFCRESGLRTR |
| | | TGSNIDCEKLRRRFSSPHFMVEVKGDLTAK |
| | | KMVLALLELAQQDHGALDCCVVVILSHGCQ |
| | | ASHLQFPGAVYGTDGCPVSVEKIVNIFNGT |
| | | SCPSLGGKPKLFFIQACGGEQKDHGFEVAS |
| | | TSPEDESPGSNPEPDATPFQEGLRTFDQLD |
| | | AISSLPTPSDIFVSYSTFPGFVSWRDPKSG |
| | | SWYVETLDDIFEQWAHSEDLQSLLLRVANA |
| | | VSVKGIYKQMPGCFNFLRKKLFFKTS |
| 8 | Truncated | GFGDVGALESLRGNADLAYILSMEPCGHCL |
| | caspase 9 | IINNVNFCRESGLRTRTGSNIDCEKLRRRF |
| | (CARD | SSLHFMVEVKGDLTAKKMVLALLELAQQDH |
| | domain | GALDCCVWILSHGCQASHLQFPGAVYGTDG |
| | removed) | CPVSVEKIVNIFNGTSCPSLGGKPKLFFIQ |
| | | ACGGEQKDHGFEVASTSPEDESPGSNPEPD |
| | | ATPFQEGLRTFDQLDAISSLPTPSDIFVSY |
| | | STFPGFVSWRDPKSGSWYVETLDDIFEQWA |
| | | HSEDLQSLLLRVANAVSVKGIYKQMPGCFN |
| | | FLRKKLFFKTS |
| 9 | FKBP12 domain | MGVQVETISPGDGRTFPKRGQTCVVHYTGM |
| | | LEDGKKFDSSRDRNKPFKFMLGKQEVIRGW |
| | | EEGVAQMSVGQRAKLTISPDYAYGATGHPG |
| | | IIPPHATL VFDVELLKLE |
| 10 | wild-type FRB | MASRILWHEMWHEGLEEASRLYFGERNVKG |
| | segment | MFEVLEPLHAMMERGPQTLKETSFNQAYGR |
| | | DLMEAQEWCRKYMKSGNVKDLTQAWDLYYH |
| | | VFRRISKLES |
| 11 | FRB with | MASRILWHEMWHEGLEEASRLYFGERNVKG |
| | T2098L | MFEVLEPLHAMMERGPQTLKETSFNQAYGR |
| | substitution | DLMEAQEWCRKYMKSGNVKDLLQAWDLYYH |
| | (eqiv | VFRRISKLES |
| | positions | |
| | in WT | |
| | mTOR; | |
| | binds | |
| | AP21967) | |
| 12 | FRB with | MASRILWHEMWHEGLEEASRLYFGERNVKG |
| | T2098H and | MFEVLEPLHAMMERGPQTLKETSFNQAYGR |
| | F2101W | DLMEAQEWCRKYMKSGNVKDLHQAFDLYYH |
| | substitutions | VFRRISKLES |
| | (eqiv | |
| | positions | |
| | in | |
| | WT mTOR; | |
| | reduced | |
| | rapamycin | |
| | affinity) | |
| 13 | FRB segment of | MASRILWHEMWHEGLEEASRLYFGERNVKG |
| | mTOR with a K | MFEVLEPLHAMMERGPQTLKETSFNQAYGR |
| | to P | DLMEAQEWCRKYMKSGNVPDLTQAWDLYYH |
| | substitution | VFRRISKLES |
| | at position | |
| | equivalent to | |
| | 2,095 | |
| | (reduced | |
| | rapamycin | |
| | affinity) | |
| 52 | Inducible | MLEGVQVETISPGDGRTFPKRGQTCVVHYT |
| | caspase 9 | GMLEDGKKVDSSRDRNKPFKFMLGKQEVIR |
| | | GWEEGVAQMSVGQRAKLTISPDYAYGATGH |
| | | PGIIPPHATLVFDVELLKLESGGGSGVDGF |
| | | GDVGALESLRGNADLAYILSMEPCGHCLII |
| | | NNVNFCRESGLRTRTGSNIDCEKLRRRFSS |
| | | LHFMVEVKGDLTAKKMVLALLELAQQDHGA |
| | | LDCCVVVILSHGCQASHLQFPGAVYGTDGC |
| | | PVSVEKIVNIFNGTSCPSLGGKPKLFFIQA |
| | | CGGEQKDHGFEVASTSPEDESPGSNPEPDA |
| | | TPFQEGLRTFDQLDAISSLPTPSDIFVSYS |
| | | TFPGFVSWRDPKSGSWYVETLDDIFEQWAH |
| | | SEDLQSLLLRVANAVSVKGIYKQMPGCFNF |
| | | LRKKLFFKTSVDYPYDVPDYALD |
| 53 | Inducible | MVEGVQVETISPGDGRTFPKRGQTCVVHYT |
| | caspase 9 | GMLEDGKKVDSSRDRNKPFKFMLGKQEVIR |
| | | GWEEGVAQMSVGQRAKLTISPDYAYGATGH |
| | | PGIIPPHATLVFDVELLKLESGGGSGVDGF |
| | | GDVGALESLRGNADLAYILSMEPCGHCLII |
| | | NNVNFCRESGLRTRTGSNIDCEKLRRRFSS |
| | | LHFMVEVKGDLTAKKMVLALLELAQQDHGA |
| | | LDCCVVVILSHGCQASHLQFPGAVYGTDGC |
| | | PVSVEKIVNIFNGTSCPSLGGKPKLFFIQA |
| | | CGGEQKDHGFEVASTSPEDESPGSNPEPDA |
| | | TPFQEGLRTFDQLDAISSLPTPSDIFVSYS |
| | | TFPGFVSWRDPKSGSWYVETLDDIFEQWAH |
| | | SEDLQSLLLRVANAVSVKGIYKQMPGCFNF |
| | | LRKKLFFKTS |
| 54 | Self- | MGFGDVGALESLRGNADLAYILSMEPCGHC |
| | activating | LIINNVNFCRESGLRTRTGSNIDCEKLRRR |
| | caspase 9 | FSSLHFMVEVKGDLTAKKMVLALLELAQQD |
| | | HGALDCCVVVILSHGCQASHLQFPGAVYGT |
| | | DGCPVSVEKIVNIFNGTSCPSLGGKPKLFF |
| | | IQACGGEQKDHGFEVASTSPEDESPGSNPE |
| | | PDATPFQEGLRTFDQLDAISSLPTPSDIFV |
| | | SYSTFPGFVSWRDPKSGSWYVETLDDIFEQ |
| | | WAHSEDLQSLLLRVANAVSVKGIYKQMPCI |
| | | VSMLRKKLFFKTS |
| 55 | Self- | MLEGVQVETISPGDGRTFPKRGQTCVVHYT |
| | activating | GMLEDGKKVDSSRDRNKPFKFMLGKQEVIR |
| | caspase 9 | GWEEGVAQMSVGQRAKLTISPDYAYGATGH |
| | | PGIIPPHATLVFDVELLKLESGGGSGVDGF |
| | | GDVGALESLRGNADLAYILSMEPCGHCLII |
| | | NNVNFCRESGLRTRTGSNIDCEKLRRRFSS |
| | | LHFMVEVKGDLTAKKMVLALLELAQQDHGA |
| | | LDCCVVVILSHGCQASHLQFPGAVYGTDGC |
| | | PVSVEKIVNIFNGTSCPSLGGKPKLFFIQA |
| | | CGGEQKDHGFEVASTSPEDESPGSNPEPDA |
| | | TPFQEGLRTFDQLDAISSLPTPSDIFVSYS |
| | | TFPGFVSWRDPKSGSWYVETLDDIFEQWAH |
| | | SEDLQSLLLRVANAVSVKGIYKQMPCIVSM |
| | | LRKKLFFKTSVDYPYDVPDYALD |

A cytotoxic protein disclosed herein can comprise, consist essentially of, or consist of an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to any one of SEQ ID NOs: 3-13 and 52-55.

A cytotoxic protein disclosed herein can comprise, consist essentially of, or consist of an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to at least 100 consecutive amino acids of any one of SEQ ID NOs: 3-13 and 52-55.

A cytotoxic protein disclosed herein can comprise, consist essentially of, or consist of an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to at least 250 consecutive amino acids of any one of SEQ ID NOs: 3-13 and 52-55.

A cytotoxic protein disclosed herein can comprise, consist essentially of, or consist of an amino acid sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity or sequence similarity to any one of SEQ ID NOs: 3-13 and 52-55.

In some embodiments, a cytotoxic protein comprises, consists essentially of, or consists of an amino acid sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, about 99.5%, or about 100% sequence identity or sequence similarity to any one of SEQ ID NOs: 3-13 and 52-55.

In some embodiments, the cytotoxic protein comprises, consists essentially of, or consists of the amino acid sequence of any one of SEQ ID NOs: 3-13 and 52-55.

In some embodiments, the cytotoxic protein comprises an amino acid sequence with one or more insertions, deletions, and/or substitutions relative to any one of SEQ ID NOs: 3-13 and 52-55.

For example, the cytotoxic protein can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions relative to any one of SEQ ID NOs: 3-13 and 52-55.

In some embodiments, the cytotoxic protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any one of SEQ ID NOs: 3-13 and 52-55.

In some embodiments, the cytotoxic protein comprises 1, 2, 3, 4, 5, 6, 7, 8, 9, 10, 11, 12, 13, 14, 15, 16, 17, 18, 19, 20, 25, 30, 35, 40, 45, or 50 amino acid insertions relative to any one of SEQ ID NOs: 3-13 and 52-55.

The one or more insertions can be at the N-terminus, C-terminus, within the amino acid sequence, or a combination thereof. The one or more insertions can be contiguous, non-contiguous, or a combination thereof.

In some embodiments, the cytotoxic protein comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid deletions relative to any one of SEQ ID NOs: 3-13 and 52-55.

In some embodiments, the cytotoxic protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any one of SEQ ID NOs: 3-13 and 52-55.

In some embodiments, the cytotoxic protein comprises 1, 2, 3, 4, 5, 6, 7, 8, 9, 10, 11, 12, 13, 14, 15, 16, 17, 18, 19, 20, 25, 30, 35, 40, 45, or 50 amino acid deletions relative to any one of SEQ ID NOs: 3-13 and 52-55.

The one or more deletions can be at the N-terminus, C-terminus, within the amino acid sequence, or a combination thereof. The one or more deletions can be contiguous, non-contiguous, or a combination thereof.

In some embodiments, the cytotoxic protein comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid substitutions relative to any one of SEQ ID NOs: 3-13 and 52-55.

In some embodiments, the cytotoxic protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any one of SEQ ID NOs: 3-13 and 52-55.

In some embodiments, the cytotoxic protein comprises 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to any one of SEQ ID NOs: 3-13 and 52-55.

The one or more substitutions can be at the N-terminus, C-terminus, within the amino acid sequence, or a combination thereof. The one or more substitutions can be contiguous, non-contiguous, or a combination thereof.

A cytotoxic protein can be, comprise, consist essentially of, or consist of BAX, DFF40, Herpes Simplex Virus Thymidine Kinase (HSV-TK), cytosine deaminase, or a catalytic domain thereof.

A cytotoxic protein can be, comprise, consist essentially of, or consist of an inducible (e.g., rapamycin-inducible) BAX, DFF40, Herpes Simplex Virus Thymidine Kinase (HSV-TK), cytosine deaminase, or a catalytic domain thereof.

A cytotoxic protein can be, comprise, consist essentially of, or consist of a non-inducible BAX, DFF40, Herpes Simplex Virus Thymidine Kinase (HSV-TK), cytosine deaminase, or a catalytic domain thereof.

A cytotoxic protein can be, comprise, consist essentially of, or consist of a self-activating BAX, DFF40, Herpes Simplex Virus Thymidine Kinase (HSV-TK), cytosine deaminase, or a catalytic domain thereof.

DNA fragmentation factor (DFF) can be a complex of the DNase DFF40 (CAD) and its chaperone/inhibitor DFF45 (ICAD-L). In its inactive form, DFF can be a heterodimer composed of a 45 kDa chaperone inhibitor subunit (DFF45 or ICAD), and a 40 kDa latent endonuclease subunit (DFF40 or CAD). Upon caspase-3 cleavage of DFF45, DFF40 forms active endonuclease homo-oligomers. Active DFF can induce DNA fragmentation. DNA binding by DFF is mediated by the nuclease subunit, which can also form stable DNA complexes after release from DFF. The nuclease subunit is inhibited in DNA cleavage but not in DNA binding. DFF45 can also be cleaved and inactivated by caspase-7. The cleaved DFF45 fragments dissociate from DFF40, allowing DFF40 to oligomerize, forming a large complex that cleaves DNA by introducing double strand breaks. Histone H1 confers DNA binding ability to DFF and stimulates the nuclease activity of DFF40.

Thymidine kinase (TK) is an ATP-thymidine 5′-phosphotransferase that can be present in living cells as well as in certain viruses including herpes simplex virus (HSV), varicella zoster virus (VZV), and Epstein-Barr virus (EBV). Thymidine kinase converts deoxythymidine into deoxythymidine 5′-monophosphate (TMP), which is phosphorylated to deoxythymidine diphosphate and to deoxythymidine triphosphate by thymidylate kinase and nucleoside diphosphate kinase, respectively. Deoxythymidine triphosphase can be incorporated into cellular DNA by DNA polymerases and viral reverse transcriptases. When incorporated into DNA, certain dNTP analogs, such as synthetic analogues of 2′-deoxy-guanosine (e.g., Ganciclovir), cause the premature termination of DNA synthesis, which triggers cellular apoptosis. In some embodiments, the polynucleotide constructs and systems of the present disclosure can employ a transgene that encodes HSV-TK. Following the administration to a human of a polynucleotide construct or system employing a transgene encoding HSV-TK, an analogue of a 2′-deoxy-nucleotide, such as 2′-deoxy-guanosine, can be administered to the human. The HSV-TK efficiently converts the 2′-deoxy-nucleotide analogue into a dNTP analogue, which when incorporated into the DNA can induce apoptosis in the target cell.

Cytosine deaminase (CD) catalyzes the hydrolytic conversion in DNA of cytosine to uracil and ammonia. If a CD-modified site is recognized by an endonuclease, the phosphodiester bond is cleaved and, in a normal cell, is repaired by incorporating a new cytosine. In the presence of 5-fluorocytosine (5-FC), cytosine deaminase converts 5-FC into 5-fluorouracil (5-FU), which can inhibit target cell growth. Transgenic expression of CD in a target cell, therefore, can reduce the growth and/or survival of the target cell.

In some embodiments the cytotoxic protein induces cell death by activating an endogenous cell death pathway (e.g., activating one or more caspases).

Non-limiting examples of cytotoxic proteins that can be used are disclosed in US20170354682A1; WO 2008/154644; US2011/0286980; US20230065562A1; Stavrou, Mol. Therapy 26(5):1266-1276 (2018); Xie et al., Cancer Res 61(18):186-91 (2001); Carlotti et al., Cancer Gene Ther 12(7):627-39 (2005); Lowe et al., Gene Ther 8(18):1363-71 (2001); and Shariat et al., Cancer Res 61(6):2562-71 (2001); Liu et al., J Biol Chem 274(20):13836-40 (1999); Shah et al., Genesis 45(4):104-199 (2007); Straathof et al., Blood 105(11):4247-4254 (2005); Carlotti et al., Cancer Gene Ther 12(7):627-39 (2005); Clackson et al., Proc. Natl. Acad. Sci. U.S.A. 95:10437-10442 (1998); Gargett T, Brown M P. The inducible caspase-9 suicide gene system as a “safety switch” to limit on-target, off-tumor toxicities of chimeric antigen receptor T cells. Front Pharmacol. 2014 Oct. 28; 5:235; Zhou X, Brenner M K. Improving the safety of T-Cell therapies using an inducible caspase-9 gene. Exp Hematol. 2016 November; 44(11):1013-1019; Falcon et al. Combinatorial suicide gene strategies for the safety of cell therapies. Front Immunol. 2022 Sep. 14; 13:975233; and Bouquet et al. “RapaCaspase-9-based suicide gene applied to the safety of IL-1RAP CAR-T cells.” Gene Therapy (2023): 1-8; each of which is incorporated herein by reference in its entirety.

### C. Extracellular Matrix Remodeling Factor

A polynucleotide construct disclosed herein can comprise a transgene that encodes an extracellular matrix remodeling factor. An extracellular matrix remodeling factor can function in restructuring the extracellular matrix, for example, after apoptosis of adipocytes. The transgene that encodes an extracellular matrix remodeling factor can be under regulatory control of the same promoter or a different promoter than the cytotoxic protein. The transgene that encodes an extracellular matrix remodeling factor and the transgene that encodes the cytotoxic protein can be part of a fusion protein and separated by a 2A cleavable or self-cleaving linker disclosed herein.

In some embodiments, the extracellular matrix remodeling factor is a matrix metalloproteinase, for example, MMP-1, MMP-3, MMP-8, MMP-10, MMP-11, MMP-12, MMP-13, MMP-21, MMP-27, MMP-7, MMP-26, MMP-2, MMP-9, MMP-14, MMP-15, MMP-16, MMP-17, MMP-24, or MMP-25. In some embodiments, the extracellular matrix remodeling factor is a disintegrin and metalloproteinase with thrombospondin motifs (ADAMTS), for example, ADAMTS-1, ADAMTS-4, ADAMTS-5, ADAMTS-8, ADAMTS-9, ADAMTS-15, ADAMTS-20, ADAMTS-2, ADAMTS-3, ADAMTS-14, ADAMTS-13, ADAMTS-7, ADAMTS-12, ADAMTS-6, ADAMTS-10, ADAMTS-16, ADAMTS-17, ADAMTS-18, or ADAMTS-19. In some embodiments, the extracellular matrix remodeling factor is a serine proteinase, for example, plasmin or cathepsin-G. In some embodiments, the extracellular matrix remodeling factor is a cysteine protease, for example, cathepsin B or cathepsin L.

### D. Safety Element

A polynucleotide construct disclosed herein can comprise a safety element to regulate expression or activity of a transgene, therapeutic protein, or cytotoxic protein disclosed herein in appropriate cells (e.g., adipocytes or white adipocytes), and/or limit expression or activity of a transgene, therapeutic protein, or cytotoxic protein in control cells (e.g., a non-adipocyte such as a myocyte, hepatocyte, osteocyte, erythrocyte, neuron, leukocyte, lymphocyte, monocyte, or fibroblast, hematopoietic lineage cells, central or peripheral nervous system cells, dorsal root ganglia neurons, or a cell that is not a white adipocyte, e.g., a brown adipocyte) or tissue(s) (e.g., skeletal muscle, cardiac muscle, pancreas, gastrointestinal tract).

In some embodiments, the safety element is a regulatory RNA, for example, a small-interfering RNA (siRNA). In some embodiments, the siRNA can be identical, substantially identical, to a portion of any one of SEQ ID NOs: 2 and 14-17 or a variant thereof that is at least about 80%, at least about 85%, at least about 90%, at least about 95%, at least about 98%, or at least about 99% identical to a portion of any one of SEQ ID NOs: 2 and 14-17 (which can include, e.g., a complement, reverse complement, and/or RNA equivalent sequence thereof). In some embodiments, the regulatory RNA can be a microRNA (miRNA). In some embodiments, the miRNA can be identical to or substantially identical to a portion of any one of SEQ ID NOS: 2 and 14-17 or a variant thereof that is at least about 60%, at least about 65%, at least about 70%, at least about 75%, at least about 80%, at least about 85%, at least about 90%, at least about 95%, at least about 98%, or at least about 99% identical to a portion of any one of SEQ ID NOs: 2 and 14-17 (which can include, e.g., a complement, reverse complement, and/or RNA equivalent sequence thereof). The portion can be at least 10, at least 15, at least 20, or at least 30 nucleotides. The portion can be at most 20, at most 30, at most 50, or at most 100 nucleotides.

In some embodiments, the safety element comprises a target site of a regulatory RNA, miRNA or siRNA, for example, in an untranslated region.

MicroRNAs (miRNAs) are small noncoding RNA molecules that regulate gene expression. They function by binding to complementary sequences on target messenger RNAs (mRNAs), leading to either mRNA degradation or inhibition of translation. This interaction with mRNA occurs primarily in the 3′ untranslated region (3′ UTR). In gene therapy, miRNAs have been utilized to refine transgene specificity and minimize off-target effects. By incorporating tissue-specific miRNAs target sites (miRTs) into the 3′ UTR of transgene mRNAs, expression can be selectively inhibited in specific tissues where those miRNAs are abundant, enhancing the precision of gene therapy applications and adipocyte-targeting systems and methods disclosed herein. Examples and characteristics of tissue-specific miRNA target sequences that can be used in compositions and methods disclosed herein are provided in TABLE 3, and illustrative sequences are provided in TABLE 4.

In some embodiments, the safety element or a site targeted by the safety element comprises or is identical to or substantially identical to a portion, complement, or reverse complement, or RNA equivalent of any one of SEQ ID NOs: 73-90 or a portion of a variant thereof that is at least about 60%, at least about 65%, at least about 70%, at least about 75%, at least about 80%, at least about 85%, at least about 90%, at least about 95%, at least about 98%, or at least about 99% identical to any one of SEQ ID NOs: 73-90 (which can include, e.g., a complement, reverse complement, and/or RNA equivalent sequence thereof). The portion can be at least 10, at least 15, at least 20, or at least 30 nucleotides. The portion can be at most 20, at most 30, at most 50, or at most 100 nucleotides.

A polynucleotide construct or system disclosed herein can use one safety element or a plurality of safety elements, e.g., two, three, four, five, or more regulatory RNAs and/or sites targeted by regulatory RNAs.

TABLE 3

| | De-targeted | |
|---|---|---|
| miRNA/target site | cell/tissue | Illustrative characteristics |
| miR142-3p | Hematopoietic lineage | 100-fold reduction of transgene expression in |
| | | intra- and extravascular hematopoietic |
| | | lineages & expression maintained in |
| | | non-hemtopoietic cells |
| | | Silencing gene expression in antigen |
| | | presenting cells to decrease host immune |
| | | responses |
| miR206-3p | Skeletal muscle | Decreases gene expression in muscle tissue |
| miR206-3g | | when using tissue-specific promoter |
| miR206 | | Skeletal muscle-specific gene silencing, while |
| | | full expression in heart tissue due to singel- |
| | | nucleotide substitution into the miR-206TS |
| | | 112-fold reduction in gene expression in |
| | | skeletal muscle |
| miR122a | Hepatocyte | Decreases gene expression in target tissue |
| | | when using tissue-specific promoter |
| | | 50-70-fold decrease in gene expression in liver |
| | | Abolished transgene expression in the liver, |
| | | without affecting expression in the heart and |
| | | without side effects |
| miR1d | Heart & skeletal | Transgene expression in heart almost |
| miR1 | muscle | completely abrogated after IV delivery |
| | | 100-fold and 50-fold decreased transgene |
| | | expression in heart and muscle, respectively |
| miR181 | HSC-derived blood | Specific targeting of hTERT⁺ cancer cells but |
| | cells | not hTERT⁺ HSCs |
| miR124t | Neurons | Restricted transgene expression in astrocytes |
| miR124 | CNS | and not in neurons |
| | | Suppressed viral replication in CNS of an |
| | | oncolytic virus |
| miR183 | Dorsal root ganglion | Reduced transgene expression and toxicity in |
| | neurons | DRG neurons after injection in CNS |
| miR133 & miR208 | Heart | Suppressed viral replication in cardiac tissue of |
| | | an oncolytic virus to avoid cardiomyositis |
| miR148a | Pancreas & GI tract | Suppresed viral replication in miR-148a |
| | | transfected cells |

TABLE 4: illustrative miRNA sequences. In some cases oligonucleotides are provide that can be annealed to generate a sequence containing multiple copies of the miR-TS. Underlining indicates the miR target site (TS) that is complementary to the tissue-specific miRNA.

TABLE 4

| miRNA | | SEQ ID |
|---|---|---|
| name | Sequence | NO: |
| 4xmiR142- | CTAGAGTCGACTCCATAAAGTAGGAAACACTACACGATTCCA | 73 |
| 3p | TAAAGTAGGAAACACTACAACCGGT | |
| | TGTAGTGTTTCCTACTTTATGGAATCGTGTAGTGTTTCCTACT | 74 |
| | TTATGGAGTCGACT | |
| | TCCATAAAGTAGGAAACACTACATCACTCCATAAAGTAGGAA | 75 |
| | ACACTACAC | |
| | TCGAGTGTAGTGTTTCCTACTTTATGGAGTGATGTAGTGTTTC | 76 |
| | CTACTTTATGGAACCGGT | |
| 4xmiR206p | GATCCACATGTCGTACGACCACACACTTCCTTACATTCCACGA | 77 |
| | TACCACACACTTCCTTACATTCCAGTTTAAAC | |
| | TGGAATGTAAGGAAGTGTGTGGTATCGTGGAATGTAAGGAA | 78 |
| | GTGTGTGGTCGTACGACATGTG | |
| | ACCACACACTTCCTTACATTCCACGATACCACACACTTCCTTA | 79 |
| | CATTCCAACCGGTACATGTA | |
| | GATCTACATGTACCGGTTGGAATGTAAGGAAGTGTGTGGTAT | 80 |
| | CGTGGAATGTAAGGAAGTGTGTGGTGTTTAAAC | |
| miR206-3g | AGGCCTCCACACACTTCCTTACATTGCATCTAGGCCACACACT | 81 |
| | TCCTTACATTGCATCTAGGCCACACACTTCCTTACATTGCATCT | |
| | AGA | |
| 6xmiR206 | TCTAGACCACACACTTCCTTACATTCCAGGATCCCCACACACT | 82 |
| | TCCTTACATTCCACGTACGCCACACACTTCCTTACATTCCAAC | |
| | GCGTCCACACACTTCCTTACATTCCACACGTGCCACACACTTC | |
| | CTTACATTCCAGCATGCCCACACACTTCCTTACATTCCAGCGG | |
| | CCGC | |
| miR 122a | TCGACACAAACACCATTGTCACACTCCAC | 83 |
| | AATTGTGGAGTGTGACAATGGTGTTTGTG | 84 |
| | TAATTCTAGATCGCGAACAAACACCATTGTCACACTCCAGTAC | 85 |
| | TA | |
| miR1d | GGCCGGCCAATACATACTTCTTTACATTCCACGATAATACATAC | 86 |
| | TTCTTTACATTCCAACCGGTAATACATACTTCTTTACATTCCAT | |
| | CACAATACATACTTCTTTACATTCCAGGCCGGCC | |
| miR181a | ACTCACCGACAGCGTTGAATGTT | 87 |
| miR124t | GATCCCTCGAGGCTGAGGTCACAGTGTGCCTTAGAGAGCTGA | 88 |
| | GTCACAGTGTGCCTTCTCGAGGTCGACG | |
| | GATCCGTCGACCTCGAGAAAGGCACACTGTGACCTCAGCTCT | 89 |
| | CAAAGGCACACTGTGACCTCAGCCTCGAG | |
| 3xmiR | ACAAAGTTCTGTAGTGCACTGAGAGCTTGACAAAGTTCTGTA | 90 |
| 148a | GTGCACTGAGAGCTTGACAAAGTTCTGTAGTGCACTGA | |

Non-limiting examples of regulatory RNAs and regulatory RNA target sites that can be utilized are disclosed in Brown et al. Endogenous microRNA regulation suppresses transgene expression in hematopoietic lineages and enables stable gene transfer. Nat Med. 2006 May; 12(5):585-91; Brown B et al. A microRNA-regulated lentiviral vector mediates stable correction of hemophilia B mice. Blood. 2007 Dec. 15; 110(13):4144-52; Wolff et al. Effect of tissue-specific promoters and microRNA recognition elements on stability of transgene expression after hydrodynamic naked plasmid DNA delivery. Hum Gene Ther. 2009 April; 20(4):374-88; Geisler et al. Application of mutated miR-206 target sites enables skeletal muscle-specific silencing of transgene expression of cardiotropic AAV9 vectors. Mol Ther. 2013 May; 21(5):924-33; Greig et al. Intramuscular injection of AAV8 in mice and macaques is associated with substantial hepatic targeting and transgene expression. PLoS One. 2014 Nov. 13; 9(11):e112268; Qiao C et al. Liver-specific microRNA-122 target sequences incorporated in AAV vectors efficiently inhibits transgene expression in the liver. Gene Ther. 2011 April; 18(4):403-10; Geisler et al. microRNA122-regulated transgene expression increases specificity of cardiac gene transfer upon intravenous delivery of AAV9 vectors. Gene Ther. 2011 February; 18(2):199-209; Trepel et al. Treatment of multifocal breast cancer by systemic delivery of dual-targeted adeno-associated viral vectors. Gene Ther. 2015 October; 22(10):840-7; Xie et al. MicroRNA-regulated, systemically delivered rAAV9: a step closer to CNS-restricted transgene expression. Mol Ther. 2011 March; 19(3):526-35; Won et al. Targeted anticancer effect through microRNA-181a regulated tumor-specific hTERT replacement. Cancer Lett. 2015 Jan. 28; 356(2 Pt B):918-28; Colin et al. Engineered lentiviral vector targeting astrocytes in vivo. Glia. 2009 Apr. 15; 57(6):667-79; Ruiz et al. MicroRNA-Detargeted Mengovirus for Oncolytic Virotherapy. J Virol. 2016 Mar. 28; 90(8):4078-4092; Hordeaux et al. MicroRNA-mediated inhibition of transgene expression reduces dorsal root ganglion toxicity by AAV vectors in primates. Sci Transl Med. 2020 Nov. 11; 12(569):eaba9188; and Baertsch et al. MicroRNA-mediated multi-tissue detargeting of oncolytic measles virus. Cancer Gene Ther. 2014 September; 21(9):373-80, each of which is incorporated herein by reference for such disclosure.

In some embodiments, the safety element can induce transcriptional repression. In some embodiments, the safety element can be a transcriptional repressor, such as a Snail (SNAI1) protein, a B cell lymphoma 6 (BCL6) protein, a Nuclear Factor of Activated T-Cells, Cytoplasmic-4 (NFATC4) protein, Activating Transcription Factor-3 (ATF-3) protein.

In some embodiments, expression of the safety element (or a regulatory RNA targeting a target site in the safety element) is driven by a non-adipocyte promoter (e.g., a promoter that is not active, substantially inactive, or minimally active in adipocyte, but can be active in one or more other cell types, such as control cell type(s) disclosed herein). The promoter can be endogenous, exogenous, or engineered. In some embodiments, a regulatory RNA targeting the target site in the safety element is endogenously expressed in one or more control (e.g., non-adipocyte) tissue(s). In some embodiments, the non-adipocyte promoter can be a hepatocyte-specific promoter, for example, an albumin (ALB) promoter, a α-fetoprotein (AFP) promoter, or a transthyretin (TTR) promoter. In some embodiments, the non-adipocyte promoter can be a leukocyte-specific promoter, for example, a lymphocyte-specific protein-1 (LSP1) promoter, or a CD11a (ITGAL) promoter.

### E. Additional Aspects of Polynucleotide Constructs

A polynucleotide construct can comprise a poly-adenylation (poly(A)) signal that can direct mRNA 3′ end formation and addition of the poly(A) to the 3′ of the mRNA. In some embodiments, the poly(A) signal can be or comprise a BGH pA (CpG free-long). In some embodiments, the poly(A) signal can be or comprise a beta globin poly(A) signal.

A polynucleotide construct can lack an origin of replication. A polynucleotide construct can comprise an origin of replication.

A polynucleotide construct can comprise an antibiotic resistance genes that can allow for the positive selection of cells (e.g., bacterial cells) that contain the disclosed polynucleotide construct during the vector purification process. In some embodiments, a polynucleotide construct lacks an antibiotic resistance gene.

A polynucleotide construct can comprise a basis of mobility (BOM) region that can function in bacterial conjugation and horizontal transfer of extra-chromosomal plasmids. In some embodiments, a polynucleotide construct lacks a BOM region.

In some embodiments, a polynucleotide or expression construct disclosed herein comprises natural, synthetic, and/or artificial nucleotide analogues or bases. In some embodiments, the synthetic or artificial nucleotide analogues or bases comprise modifications at one or more of a deoxyribose moiety, ribose moiety, phosphate moiety, nucleoside moiety, or a combination thereof.

In some embodiments, a transgene (e.g., encoding a cytotoxic protein, extracellular matrix remodeling factor, or safety element disclosed herein) is codon optimized. In some embodiments, a transgene (e.g., encoding a cytotoxic protein, extracellular matrix remodeling factor, or safety element) is a codon-optimized version of a transgene or transgene sequence disclosed herein.

Codon optimization can be used to increase expression, e.g., in human cells, such as adipocytes. Codon-optimized coding regions can be designed by various different methods, including methods that are published, publicly available, or commercially available. Since the genetic code is degenerate (i.e., each amino acid can be coded by on average three different codons), the DNA sequence can be modified by synonymous nucleotide substitutions without altering the amino acid sequence of the encoded protein.

Illustrative codon optimizing methods are described, e.g., in U.S. Pat. Nos. 7,561,972; 7,561,973; and 7,888, 112, and International Patent Application Pub. No. WO 2015/012924, which are incorporated by reference for such disclosure. A transgene sequence that codes for a product (e.g., cytotoxic protein, extracellular matrix remodeling factor, or safety element disclosed herein) can be modified with synonymous codon sequences. Codon optimization can comprise use of any suitable available codon frequency table, including any disclosed in or referred to in U.S. Pat. Nos. 7,561,972; 7,561,973; and 7,888,112, and International Patent Application Pub. No. WO 2015/012924, which are incorporated by reference for such disclosure. Codons can be selected for a particular tissue or cell type, e.g., white adipose tissue or white adipocytes.

In some embodiments, the entire length of the open reading frame (ORF) for the product is modified. In some embodiments, only a fragment of the ORF is altered. By using one of these methods, one can apply the codon frequencies to any given polypeptide sequence, and produce a nucleic acid fragment of a codon-optimized coding region which encodes the polypeptide.

In some embodiments, a polynucleotide construct or polynucleotide disclosed herein can be or can comprise single stranded DNA.

A polynucleotide construct can be assembled by a variety of methods, e.g., by automated solid-phase synthesis. A polynucleotide construct can be constructed using standard solid-phase DNA/RNA synthesis. A polynucleotide construct can also be constructed using a synthetic procedure. A polynucleotide construct can be synthesized manually or in a fully automated fashion. A polynucleotide construct can be a recombinant nucleic acid. In some cases, a synthetic procedure may comprise 5′-hydroxyl oligonucleotides that can be initially transformed into corresponding 5′-H-phosphonate mono esters, subsequently oxidized in the presence of imidazole to activated 5′-phosphorimidazolidates, and finally reacted with pyrophosphate on a solid support. This procedure may include a purification step after the synthesis such as PAGE, HPLC, MS, or any combination thereof. Polynucleotides can be purchased commercially.

### II. TARGET CELLS

A target cell in which a transcriptional promoter disclosed herein is active can be an adipocyte, such as a white adipocyte (e.g. adipose/fat cell). White adipocytes can be formed as a result of storing excess calories.

In some embodiments, the target cell is not a brown adipocyte. Brown adipocytes can generate heat by burning calories, for example in a process of non-shivering thermogenesis. In some embodiments, the target cell can be a single adipocyte. A plurality of adipocytes can be target cells, e.g., as a population of target cells. In some embodiments, the target cell includes a brown adipocyte.

A target cell or a plurality of target cells can be population of adipocytes in an adipose tissue. In some embodiments, the target cell population can be a population of adipocytes in a fatty tumor (e.g. lipoma).

A target cell or a plurality of target cells can be present in subcutaneous fat. A target cell or a plurality of target cells can be present in abdominal fat. A target cell or a plurality of target cells can be present in visceral fat. A target cell or a plurality of target cells can be present in hepatic fat.

A target cell or a plurality of target cells can be located in a certain part of a subject's body, for example, a body cavity or anatomical region. In some embodiments, an adipocyte or a plurality of adipocytes targeted by a composition, system, or method disclosed herein are located in a subject's abdominal cavity. In some embodiments, an adipocyte or a plurality of adipocytes targeted by a composition, system, or method disclosed herein are located in a subject's peritoneal cavity. In some embodiments, an adipocyte or a plurality of adipocytes targeted by a composition, system, or method disclosed herein are located in a subject's thoracic cavity, pelvic cavity, pleural cavity, or pericardial cavity. In some embodiments, an adipocyte or a plurality of adipocytes targeted by a composition, system, or method disclosed herein are located in a subject's legs. In some embodiments, an adipocyte or a plurality of adipocytes targeted by a composition, system, or method disclosed herein are located in a subject's arms. In some embodiments, an adipocyte or a plurality of adipocytes targeted by a composition, system, or method disclosed herein are located in a subject's torso. In some embodiments, an adipocyte or a plurality of adipocytes targeted by a composition, system, or method disclosed herein are located in a subject's back. In some embodiments, an adipocyte or a plurality of adipocytes targeted by a composition, system, or method disclosed herein are located in a subject's neck. In some embodiments, an adipocyte or a plurality of adipocytes targeted by a composition, system, or method disclosed herein are located in the subject's head. In some embodiments, an adipocyte or a plurality of adipocytes targeted by a composition, system, or method disclosed herein are located in the subject's liver. In some embodiments, an adipocyte or a plurality of adipocytes targeted by a composition, system, or method disclosed herein are located in omental, mesenteric, splenic, portal, or gonadal fat.

In some embodiments, an adipocyte or a plurality of adipocytes targeted by a composition, system, or method disclosed herein are in multiple body cavities or anatomical regions. In some embodiments, an adipocyte or a plurality of adipocytes targeted by a composition, system, or method disclosed herein are dispersed throughout a subject's body.

### III. DELIVERY VECTORS

Compositions, systems, and methods of the disclosure can comprise or utilize delivery vectors, e.g., for delivery of a polynucleotide construct or polynucleotide encoding a cytotoxic protein.

A delivery vector disclosed herein can be a lipid-based delivery vector (LDV). An LDV disclosed herein can facilitate delivery of a polynucleotide construct or polynucleotide disclosed herein, and expression of a transgene of interest after in vivo administration to a subject. For example, an LDV disclosed herein can facilitate expression of a cytotoxic protein, extracellular matrix remodeling factor, or safety element after in vivo administration. An LDV disclosed herein can utilize an effective and re-dosable delivery platform that allows high tolerability compared to alternate formulations or approaches. An LDV can comprise a lipid membrane and/or a lipid bi-layer. An LDV can exclude an enveloped viral vector.

An LDV disclosed herein can comprise one or more (for example, two or more, three or more, four or more, five or more, one, two three, four, five, or six) lipids selected from 1,2-di-O-octadecenyl-3-trimethylammonium propane (DOTMA), 1,2-dioleoyl-3-dimethylammonium-propane (DODAP), 1,2-Dioleyloxy-3-dimethylaminopropane (DODMA), 1,2-dimyristoyl-sn-glycero-3-methoxypolyethylene glycol-2000 (DMG-PEG). 2-dioleoyl-sn-glycero-3-phosphoethanolamine (DOPE) 1,2-dioleoyl-3-trimethylammonium-propane (DOTAP), Dlin-KC2-DMA (KC2), DOBAQ, 18:1 EPC, DDAB, 18:0 EPC, 18:0 DAP, L-a-dioleoyl phosphatidyl choline (DOPC), cholesterol, DF4C11PE (rac-2,3-Di[11-(F-butyl)undecanoyl) glycero-1-phosphoethanolamine), distear-4-ynoyl L-a-phosphatidylethanolamine [DS(9-yne)PE], 18:0 TAP, dipalmitoylphosphatidylcholine (DPPC), phosphatidylcholine (PC), phosphatidic acid (PA), phosphatidylglycerol (PG), phosphatidylethanolamine (PE), phosphatidylserine (PS), distearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE), and 2,3-dioleyloxy-N-[2-(sperminecarboxamido)ethyl]-N,N-dimethyl-1-propanaminium trifluoroacetate (DOSPA).

An LDV disclosed herein can comprise one or more ionizable lipids. The charge of ionizable lipids can be dependent upon the pH of the surrounding environment. Ionizable lipids include, but are not limited to, 1,2-dioleoyl-3-dimethylammonium-propane (DODAP), 1,2-dioleoyl-3-trimethylammonium-propane (DOTAP), 1,2-dioleyloxy-3-dimethylaminopropane (DODMA), 1,2-di-O-octadecenyl-3-trimethylammonium propane (DOTMA), DLin-MC3-DMA (MC3), Dlin-KC2-DMA (KC2), DOBAQ, 18:1 EPC, DDAB, 18:0 EPC, 18:0 DAP, and 18:0 TAP.

In some embodiments, ionizable lipids in an LDV disclosed herein comprise, consist essentially of, or consist of DODAP. In some embodiments, ionizable lipids in an LDV disclosed herein comprise, consist essentially of, or consist of DODMA. In some embodiments, ionizable lipids in an LDV disclosed herein comprise, consist essentially of, or consist of DODAP and DODMA. In some embodiments, ionizable lipids (e.g., in a combination or ratio referred to herein) do not include cationic lipids, such as DOTMA and/or DOTAP.

An LDV disclosed herein can comprise one or more cationic lipids. Non-limiting examples of cationic lipids include 1,2-di-O-octadecenyl-3-trimethylammonium propane (DOTMA) and 1,2-dioleoyl-3-trimethylammonium-propane (DOTAP). In some embodiments, cationic lipids are used in a sufficiently low quantity in an LDV to reduce a pro-inflammatory response to the LDV (e.g., Th1 type cytokines or type I interferon) compared to control lipid nanoparticles. In some embodiment, an LDV does not contain or substantially lacks cationic lipids. In some embodiment, an LDV does not contain or substantially lacks DOTAP. In some embodiment, an LDV does not contain or substantially lacks DOTMA. In some embodiment, an LDV does not contain or substantially lacks cationic lipids except for DOTAP. In some embodiment, an LDV does not contain or substantially lacks cationic lipids except for DOTMA. In some embodiment, an LDV does not contain or substantially lacks cationic lipids except for DOTAP and DOTMA. In some embodiments, cationic lipids in an LDV disclosed herein comprise, consist essentially of, or consist of DOTMA. In some embodiments, cationic lipids in an LDV disclosed herein comprise, consist essentially of, or consist of DOTAP. In some embodiments, cationic lipids in an LDV disclosed herein comprise, consist essentially of, or consist of DOTAP and DOTMA.

An LDV disclosed herein can comprise one or more helper lipids. Non-limiting examples of a helper lipids include 2-dioleoyl-sn-glycero-3-phosphoethanolamine (DOPE) and L-a-dioleoyl phosphatidyl choline (DOPC). In some embodiments, helper lipids in an LDV disclosed herein comprise, consist essentially of, or consist of DOPE. In some embodiment, an LDV does not contain or substantially lacks DOPE and/or DOPC.

An LDV disclosed herein can comprise one or more PEGylated lipids. A non-limiting example of a PEGylated lipid is 1,2-dimyristoyl-sn-glycero-3-methoxypolyethylene glycol-2000 (DMG-PEG). In some embodiments, PEGylated lipids in an LDV disclosed herein comprise, consist essentially of, or consist of DMG-PEG. In some embodiment, an LDV does not contain or substantially lacks DMG-PEG.

An LDV disclosed herein can comprise cholesterol. In some embodiment, an LDV does not contain or substantially lacks cholesterol.

In some embodiments, an LDV disclosed herein comprises a combination of lipids at molar ratios appropriate to reduce toxicity, immunogenicity, or a pro-inflammatory response associated with administration of the delivery vector. For example, an LDV can comprise a combination of lipids at molar ratios appropriate to reduce production of pro-inflammatory cytokines, such as tumor necrosis factor alpha (TNF-α), interferon-gamma (IFN-γ), interleukin-6 (IL-6), type I interferon, or a combination thereof associated with administration of the delivery vector. In some embodiments, an LDV disclosed herein comprises a combination of lipids at molar ratios appropriate to reduce complement activation-related pseudoallergy (CARPA). The reduction can be in comparison to, for example, control lipid nanoparticles that comprise a higher proportion of cationic lipids. The reduction can be determined by an experiment in which empty LDV or substantially non-immunogenic cargo is administered (e.g., a polynucleotide construct or polynucleotide encoding a reporter, such as GFP). In some embodiments, the combination of lipids in the LDV make the LDV or system more suitable for high dose and/or systemic administration as compared to the control lipid nanoparticles. In some embodiments, an LDV disclosed herein exhibits broader distribution upon systemic administration compared to control lipid nanoparticles or viral vectors. In some embodiments, an LDV disclosed herein exhibits reduced accumulation in the liver upon systemic administration compared to control lipid nanoparticles or viral vectors.

An LDV disclosed herein can exhibit superior properties for delivery of a DNA expression construct or polynucleotide compared to control lipid nanoparticles. For example, in some embodiments an LDV disclosed herein requires less cationic components to neutralize the anionic charge of DNA as compared to control lipid nanoparticles.

In some embodiments, an LDV disclosed herein comprises DODAP. In some embodiments, an LDV disclosed herein comprises DODMA. In some embodiments, an LDV disclosed herein comprises DODAP and DODMA.

In some embodiments, an LDV disclosed herein comprises cationic:ionizable:helper:PEGylated lipids at a molar ratio disclosed herein. The cationic lipid(s) can comprise or consist of DOTAP. The ionizable lipid(s) can comprise or consist of DODAP. The ionizable lipid(s) can comprise or consist of DODMA. The ionizable lipid(s) can comprise or consist of DOTAP. The ionizable lipid(s) can comprise or consist of DODAP and DODMA. The ionizable lipid(s) can comprise or consist of DODAP and DOTAP. The ionizable lipid(s) can comprise or consist of DODMA and DOTAP. The ionizable lipid(s) can comprise or consist of DODAP, DODMA, and DOTAP. The helper lipid(s) can comprise or consist of DOPE. The PEGylated lipid(s) can comprise or consist of DMG-PEG.

In some embodiments, the lipids of an LDV disclosed herein comprise, consist essentially of, or consist of cationic:ionizable:helper:PEGylated lipids at a molar ratio of about 24:42:30:4.

In some embodiments, the lipids of an LDV disclosed herein comprise, consist essentially of, or consist of cationic:ionizable:helper:PEGylated lipids at a molar ratio of about 6:60:30:4.

In some embodiments, the lipids of an LDV disclosed herein comprise, consist essentially of, or consist of cationic:ionizable:helper:PEGylated lipids at a molar ratio of about 0:66:30:4.

In some embodiments, the lipids of an LDV disclosed herein comprise, consist essentially of, or consist of cationic:ionizable:helper:PEGylated lipids at a molar ratio of about 3:63:30:4.

In some embodiments, the lipids of an LDV disclosed herein comprise, consist essentially of, or consist of cationic:ionizable:helper:PEGylated lipids at a molar ratio of about 49.5:24.75:23.75:2.

In some embodiments, the lipids of an LDV disclosed herein comprise, consist essentially of, or consist of cationic:ionizable:helper:PEGylated lipids at a molar ratio of about 49.5:38.5:10:2.

In some embodiments, the lipids of an LDV disclosed herein comprise, consist essentially of, or consist of cationic:ionizable:helper:PEGylated lipids at a molar ratio of about 61.7:26.3:19:3.

In some embodiments, an LDV disclosed herein comprises ionizable, cholesterol, helper, and PEGylated lipids. In some embodiments, the lipids of an LDV disclosed herein comprise, consist essentially of, or consist of ionizable:cholesterol:helper:PEGylated lipids at a molar ratio of about 49.5:38.5:10:2. In some embodiments, the lipids of an LDV disclosed herein comprise, consist essentially of, or consist of ionizable:cholesterol:helper:PEGylated lipids at a molar ratio of about 49.5:24.75:23.75:2. In some embodiments, the lipids of an LDV disclosed herein comprise, consist essentially of, or consist of ionizable:cholesterol:helper:PEGylated lipids at a molar ratio of about 61.7:26.3:19:3.

In some embodiments, the lipids of an LDV disclosed herein comprise, consist essentially of, or consist of DOTAP, DODAP, DOPE and DMG-PEG. In some embodiments, the lipids of an LDV disclosed herein comprise, consist essentially of, or consist of DOTAP, DODAP, DOPE, and DMG-PEG, at a molar ratio or in a mole percentage of about 24:42:30:4.

In some embodiments, the lipids of an LDV disclosed herein comprise, consist essentially of, or consist of DOTAP, DODMA, DOPE and DMG-PEG. In some embodiments, the lipids of an LDV disclosed herein comprise, consist essentially of, or consist of DOTAP, DODMA, DOPE, and DMG-PEG, at a molar ratio or in a mole percentage of about 24:42:30:4.

In some embodiments, the lipids of an LDV disclosed herein comprise, consist essentially of, or consist of DOTAP, DODAP, DODMA, DOPE, and DMG-PEG. In some embodiments, the lipids of an LDV disclosed herein comprise, consist essentially of, or consist of DOTAP, DODAP, DODMA, DOPE, and DMG-PEG, at a molar ratio or in a mole percentage of about 24:21:21:30:4.

In some embodiments, the lipids of an LDV disclosed herein comprise, consist essentially of, or consist of DOTAP, DODAP, DOPE and DMG-PEG. In some embodiments, the lipids of an LDV disclosed herein comprise, consist essentially of, or consist of DOTAP, DODAP, DOPE, and DMG-PEG, at a molar ratio or in a mole percentage of about 6:60:30:4 or 3:63:30:4.

In some embodiments, the lipids of an LDV disclosed herein comprise, consist essentially of, or consist of DODAP, DOPE and DMG-PEG. In some embodiments, the lipids of an LDV disclosed herein comprise, consist essentially of, or consist of DODAP, DOPE, and DMG-PEG, at a molar ratio or in a mole percentage of about 66:30:4.

In some embodiments, the lipids of an LDV disclosed herein comprise, consist essentially of, or consist of DODAP, cholesterol, DOPE, and DMG-PEG. In some embodiments, the lipids of an LDV disclosed herein comprise, consist essentially of, or consist of DODAP, cholesterol, DOPE, and DMG-PEG, at a molar ratio or in a mole percentage of about 49.5:24.75:23.75:2, about 49.5:38.5:10:2, or about 61.7:26.3:19:3.

A delivery vector, such as an LDV, can comprise a fusogenic protein to enhance fusion of the LDV with the plasma membrane of a target cell. Non-limiting examples of fusogenic proteins include a fusion associated small transmembrane (FAST) proteins, herpes simplex virus glycoprotein H, and amphiphilic anionic peptides derived from the N-terminal segment of the HA-2 subunit of influenza virus haemagglutinin, such as the IFN7 and E5CA.

A fusogenic protein can be a fusion associated small transmembrane (FAST) protein or can comprise a functional fragment of a FAST protein. A FAST protein can function receptor-independently, and at physiological pH. In some embodiments, use of a FAST protein in an LDV allows a minimal molar ratio of cationic and/or ionizable lipid to be used in order to neutralize the anionic charge of the nucleic acid, reducing or substantially eliminating the role of ionizable lipid in the delivery process (e.g., endosomal escape). In some embodiments, incorporation of a FAST protein in an LDV enhances intracellular delivery of a polynucleotide construct or polynucleotide disclosed herein. In some embodiments, use of a FAST protein in an LDV allows for omission or lower concentrations of cholesterol to be used, for example, compared to control lipid nanoparticles.

Non-limiting examples of FAST proteins are provided in WO2012040825A1, which is incorporated herein by reference for such disclosure.

The FAST protein family comprises six members named according to their molecular mass in Daltons (p10, p13, p14, p15, p16, and p22).

In some embodiments, a FAST protein utilized in a compositions, system, or method disclosed herein is a native FAST protein found in the family Reoviridae, for example, found in the genus Aquareovirus or Orthoreovirus. Non-limiting examples of orthoreoviruses include BRV (Baboon orthoreovirus), MRV (Mammalian orthoreovirus), NBV (Nelson Bay orthoreovirus), BrRV (Broome orthoreovirus), RRV (Reptilian orthoreovirus), and ARV (Avian orthoreovirus). In some embodiments, a FAST protein utilized in a compositions, system, or method disclosed herein comprises a FAST protein or domain thereof from ARV p10, BrRv p13, RRV p14, BRV p15, AqV p16, or AqV p22.

A FAST protein can comprise an N-terminal ectodomain on the extracellular or external side of the membrane or LDV. The ectodomain can be, for example, about 19-40 residues, with a myristoylation motif, or a myristate moiety on a glycine, such as a penultimate N-terminal glycine. A FAST protein ectodomain can comprise a hydrophobic patch.

A FAST protein can comprise a transmembrane domain that serves as a reverse signal-anchor sequence to direct a bitropic Nₒᵤₜ/Cᵢₙ type I topology in the membrane or LDV.

A FAST protein can comprise a C-terminal endodomain on the cytoplasmic or internal side of the membrane or LDV. A FAST protein endodomain can be, e.g., about 40-140 residues, with a membrane-destabilizing fusion peptide motif. A FAST protein endodomain can comprise a juxtamembrane polybasic motif. A FAST protein endodomain can comprise a membrane-proximal membrane curvature sensor (e.g., an amphipathic alpha helix, such as a helix-kink-helix membrane curvature sensor) to drive pore formation. A FAST protein endodomain can comprise a hydrophobic patch.

A FAST protein can comprise a proline-hinged loop. A FAST protein can comprise a type II polyproline helix. A FAST protein can comprise a conserved region that functions as a fusion peptide, e.g., by promoting rapid lipid bilayer destabilization and membrane merging. A FAST protein can comprise a palmitoylated cysteine residue. A FAST protein can comprise a hydrophobic patch.

Structure-function relationships between different FAST proteins have suggested that overlapping structural motifs of can be exchanged among certain FAST proteins to generate functional chimeric FAST fusion proteins. In some embodiments, a chimeric FAST protein disclosed herein exhibits superior fusion activity compared to a wild-type FAST protein.

Chimeric FAST proteins can be synthesized that combine the domains from different FAST proteins, such the p10, p14, and/or p15 peptides, to form a functional fusogenic protein.

A FAST protein used in an LDV disclosed herein can comprise the ectodomain from the p14 FAST protein or a functional portion thereof, the transmembrane domain from the p14 FAST protein, and the endodomain from the p15 FAST protein or a functional portion thereof. Such a FAST protein can be referred to as a “p14endo15” or “p14e15” FAST protein. In some embodiments, the fusion activity of p14e15 is mediated by the efficient p14 ectodomain fusion peptide and myristate moiety facilitating lipid mixing with the target cell membrane, followed by the p15 endodomain fusion-inducing lipid packing sensor (FLiPs) motif partitioning into the LDV membrane to promote pore formation and liposome-cell fusion activity.

A FAST protein used in an LDV disclosed herein can comprise the ectodomain from the p14 FAST protein or a functional portion thereof, the transmembrane domain from the p15 FAST protein, and the endodomain from the p14 FAST protein or a functional portion thereof. Such a FAST protein can be referred to herein as “p14TM15”.

A FAST protein used in an LDV disclosed herein can comprise the ectodomain from the p14 FAST protein or a functional portion thereof, the transmembrane domain from the p15 FAST protein, and the endodomain from the p15 FAST protein or a functional portion thereof. Such a FAST protein can be referred to as “p15ecto14” or “p15e14”.

A FAST protein used in an LDV disclosed herein can comprise p10, p13, p14, p15, p16, p22, or a chimeric fusion protein thereof. In some embodiments, the FAST protein is a p14/p15 chimera, p10/p14 chimera, or a p10/p15 chimera. In some embodiments, the FAST protein comprises: (i) the ectodomain and transmembrane domain of p14 and the endodomain of p15; (ii) the ectodomain of p14, and the transmembrane domain and endodomain of p15; or (iii) the ectodomain and endodomain of p14 and the transmembrane of p15.

In some embodiments, a FAST protein comprises an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to any one of SEQ ID NOs: 18-22.

In some embodiments, a FAST protein comprises an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to at least 40 consecutive amino acids of any one of SEQ ID NOs: 18-22.

TABLE 5 provides illustrative FAST protein sequences.

TABLE 5

| SEQ | | |
|---|---|---|
| ID | | |
| NO | Description | Sequence |
| 18 | P14 FAST | MGSGPSNFVNHAPGEAIVTGL |
| | | EKGADKVAGTISHTIWEVIAG |
| | | LVALLTFLAFGFWLFKYLQKR |
| | | RERRRQLTEFQKRYLRNSYRL |
| | | SEIQRPISQHEYEDPYEPPSR |
| | | RKPPPPPYSTYVNIDNVSAI |
| 19 | P14e15 FAST | MGSGPSNFVNHAPGEAIVTGL |
| | | EKGADKVAGTISHTIWEVIAG |
| | | LVALLTFLAFGFWLFKYLQWY |
| | | NRKSKNKKRKEQIREQIELGL |
| | | LSYGAGVASLPLLNVIAHNPG |
| | | SVISATPIYKGPCTGVPNSRL |
| | | LQITSGTAEENTRILNHDGRN |
| | | PDGSINV |
| 20 | P14 fusogenic | MGSGPSNFVNHAPGEAIVTGL |
| | protein | EKGADKVAGTISHTIWE |
| | ectodomain | |
| 21 | P15 fusogenic | KLLQWYNRKSKNKKRKEQIRE |
| | protein | QIELGLLSYGAGVASLPLLNV |
| | endodomain | IAHNPG |
| 22 | P15 fusogenic | VISATPIYKGPCTGVPNSRLL |
| | protein | QITSGTAEENTRILNHDGRNP |
| | endodomain | DGSINV |

An LDV disclosed herein can comprise one or more ionizable lipids and one or more FAST proteins (e.g., a chimeric FAST protein). In some embodiments, use of a FAST protein in an LDV allows use of certain ionizable lipids and for a favorable ratio of ionizable, helper, and PEGylated lipids.

A delivery vector can comprise a cell penetrating peptide.

A molar ratio of an ionizable lipid to a polynucleotide can be between about 2.5:1 and about 20:1. In some embodiments, the molar ratio of the ionizable lipid to the polynucleotide is about 5:1, about 7.5:1, about 10:1, or about 15:1.

In some embodiments, the molar ratio of the ionizable lipid to the polynucleotide is between about 4:1 and about 7.5:1. In some embodiments, the molar ratio of the ionizable lipid to the polynucleotide is between about 2.5:1 and about 7:5:1. In some embodiments, the molar ratio of the ionizable lipid to the polynucleotide is between about 3:1 and about 7.5:1.

In some embodiments, the molar ratio of the ionizable lipid to the polynucleotide is between about 5:1 and about 10:1.

In some embodiments, the molar ratio of the ionizable lipid to the polynucleotide is between about 5:1 and about 12:1.

In some embodiments, the molar ratio of the ionizable lipid to the polynucleotide is between about 2.5:1 and about 15:1. In some embodiments, the molar ratio of the ionizable lipid to the polynucleotide is between about 5:1 and about 15:1. In some embodiments, the molar ratio of the ionizable lipid to the polynucleotide is between about 7.5:1 and about 15:1. In some embodiments, the molar ratio of the ionizable lipid to the polynucleotide is between about 2.5:1 and about 15:1.

In some embodiments, the molar ratio of the ionizable lipid to the polynucleotide is between about 5:1 and about 20:1.

In some embodiments, the molar ratio of the ionizable lipid to the polynucleotide is about 5:1.

In some embodiments, the lipids of an LDV disclosed herein comprise, consist essentially of, or consist of DOTAP:DODAP:DOPE:DMG-PEG at a molar ratio or in a mole percentage of about 24:42:30:4, and the molar ratio of ionizable lipid to polynucleotide (e.g., pDNA) is between about 5:1 and about 10:1.

In some embodiments, the lipids of an LDV disclosed herein comprise, consist essentially of, or consist of DOTAP:DODMA:DOPE:DMG-PEG at a molar ratio or in a mole percentage of about 24:42:30:4, and the molar ratio of ionizable lipid to polynucleotide (e.g., pDNA) is between about 4:1 and about 7.5:1.

In some embodiments, the lipids of an LDV disclosed herein comprise, consist essentially of, or consist of DOTAP:DODMA:DOPE:DMG-PEG at a molar ratio or in a mole percentage of about 24:42:30:4, and the molar ratio of ionizable lipid to polynucleotide (e.g., mRNA) is between about 2.5:1 to about 7.5:1.

In some embodiments, the lipids of an LDV disclosed herein comprise, consist essentially of, or consist of DOTAP:DODAP:DODMA:DOPE:DMG-PEG at a molar ratio or in a mole percentage of about 24:21:21:30:4, and the molar ratio of ionizable lipid to polynucleotide (e.g., pDNA) is between about 3:1 to about 7.5:1.

In some embodiments, the lipids of an LDV disclosed herein comprise, consist essentially of, or consist of DOTAP:DODAP:DODMA:DOPE:DMG-PEG at a molar ratio or in a mole percentage of about 24:21:21:30:4, and the molar ratio of ionizable lipid to polynucleotide (e.g., mRNA) is between about 2.5:1 to about 7.5:1.

In some embodiments, the lipids of an LDV disclosed herein comprise, consist essentially of, or consist of DOTAP:DODAP:DOPE:DMG-PEG at a molar ratio or in a mole percentage of about 3:63:30:4, and the molar ratio of ionizable lipid to polynucleotide (e.g., pDNA) is between about 7.5:1 to about 15:1.

In some embodiments, the lipids of an LDV disclosed herein comprise, consist essentially of, or consist of DOTAP:DODAP:DOPE:DMG-PEG at a molar ratio or in a mole percentage of about 3:63:30:4, and the molar ratio of ionizable lipid to polynucleotide (e.g., pDNA) is between about 5:1 to about 12:1.

In some embodiments, the lipids of an LDV disclosed herein comprise, consist essentially of, or consist of DOTAP:DODAP:DOPE:DMG-PEG at a molar ratio or in a mole percentage of about 6:60:30:4, and the molar ratio of ionizable lipid to polynucleotide (e.g., pDNA) is between about 5:1 to about 15:1.

In some embodiments, the lipids of an LDV disclosed herein comprise, consist essentially of, or consist of DOTAP:DODAP:DOPE:DMG-PEG at a molar ratio or in a mole percentage of about 6:60:30:4, and the molar ratio of ionizable lipid to polynucleotide (e.g., mRNA) is between about 5:1 to about 15:1.

In some embodiments, the lipids of an LDV disclosed herein comprise, consist essentially of, or consist of DODAP:DOPE:DMG-PEG at a molar ratio or in a mole percentage of about 66:30:4, and the molar ratio of ionizable lipid to polynucleotide (e.g., pDNA is between about 5:1 to about 20:1).

In some embodiments, the lipids of an LDV disclosed herein comprise, consist essentially of, or consist of DODAP:DOPE:DMG-PEG at a molar ratio or in a mole percentage of about 66:30:4, and the molar ratio of ionizable lipid to polynucleotide (e.g., mRNA) is between about 5:1 to about 20:1.

In some embodiments, the lipids of an LDV disclosed herein comprise, consist essentially of, or consist of DODAP:cholesterol:DOPE:DMG-PEG at a molar ratio or in a mole percentage of about 49.5:24.75:23.75:2, and the molar ratio of ionizable lipid to polynucleotide (e.g., pDNA) is between about 5:1 to about 15:1.

In some embodiments, the lipids of an LDV disclosed herein comprise, consist essentially of, or consist of DODAP:cholesterol:DOPE:DMG-PEG at a molar ratio or in a mole percentage of about 49.5:24.75:23.75:2, and the molar ratio of ionizable lipid to polynucleotide (e.g., mRNA) is between about 2.5:1 to about 15:1.

In some embodiments, the lipids of an LDV disclosed herein comprise, consist essentially of, or consist of DODAP:cholesterol:DOPE:DMG-PEG at a molar ratio or in a mole percentage of about 49.5:38.5:10:2, and the molar ratio of ionizable lipid to polynucleotide (e.g., pDNA) is between about 5:1 to about 15:1.

In some embodiments, the lipids of an LDV disclosed herein comprise, consist essentially of, or consist of DODAP:cholesterol:DOPE:DMG-PEG at a molar ratio or in a mole percentage of about 49.5:38.5:10:2, and the molar ratio of ionizable lipid to polynucleotide (e.g., mRNA) is between about 2.5:1 to about 15:1.

In some embodiments, the lipids of an LDV disclosed herein comprise, consist essentially of, or consist of DODAP:cholesterol:DOPE:DMG-PEG at a molar ratio or in a mole percentage of about 61.7:26.3:19:3, and the molar ratio of ionizable lipid to polynucleotide (e.g., pDNA) is between about 5:1 to about 15:1.

In some embodiments, the lipids of an LDV disclosed herein comprise, consist essentially of, or consist of DODAP:cholesterol:DOPE:DMG-PEG at a molar ratio or in a mole percentage of about 61.7:26.3:19:3, and the molar ratio of ionizable lipid to polynucleotide (e.g., mRNA) is between about 2.5:1 to about 15:1.

In some embodiments, an LDV comprises a vesicle size of less than about 80 nm.

In some embodiments, an LDV is untargeted, and for example, can facilitate delivery of a polynucleotide construct to a range of cell types including target cells and non-target cells (e.g., adipocytes and non-adipocytes, such as white adipocytes and control cells that are not white adipocytes). An LDV can be capable of or configured for untargeted delivery. Specificity of expression in target cells upon non-targeted delivery can be facilitated by an expression regulatory region, such as a cell type-specific promoter.

In some embodiments, an LDV is targeted, for example, can facilitate preferential delivery of a polynucleotide construct to a target cell type or population, such as adipocytes or white adipocytes. A delivery vector can be targeted to a receptor specifically or preferentially expressed on a target cell, for example, an adipocyte-specific receptor or surface protein, or a receptor or surface protein that exhibits higher expression on adipocytes as compared to control cells. Specificity of expression in target cells upon targeted or non-targeted delivery can be further enhanced by an expression regulatory region, such as a cell type-specific promoter.

An illustrative method of making a lipid formulation to be used in generating an LDV can comprise heating lipids disclosed herein to 37° C., combining the lipids in ratios disclosed herein, mixing (e.g., vortex mixing), dehydrating the lipid mixture (e.g., in a rotavapor at 60 rpm for 2 hours, under vacuum), rehydrating with 100% ethanol, and sonicating at 37° C.

For generation of an LDV, a NanoAssemblr Benchtop microfluidics mixing instrument can be used to mix organic and aqueous solutions and make the LDVs. The organic solution can comprise or consist of the lipid formulation. The aqueous solution can comprise or consist of nucleic acid cargo, 5 FAST protein (e.g., 5 nM), and acetate buffer (e.g., 10 mM, pH 4.0). The Benchtop NanoAssemblr running protocol can comprise a total flow rate of 12 mL/min and a 3:1 aqueous to organic flow rate ratio. LDVs can be dialyzed in 8000 MWCO dialysis tubing clipped at one end. The loaded tubing can be rinsed with 5 mL of double distilled water and dialyzed in 500 mL of Dialysis Buffer (ENT1844) with gentle stirring (60 rpm) at ambient temperature for 1 hour and repeated twice with fresh Dialysis Buffer. LDVs can be concentrated using a 100 kDa Ultra filter. LDVs can be filter sterilized through 0.2 μm Acrodisc Supor filters.

Non-limiting examples of LDVs are provided in WO2022067446A1, which is incorporated herein by reference for such disclosure.

In some embodiments, a lipid-based delivery vector is or comprises a lipid nanoparticle (LNP). LNPs can be formulated with cationic and/or ionizable lipids that neutralize the anionic charge of nucleic acids and facilitate the endosomal escape of encapsulated nucleic acids through charge-mediated lipid bilayer disruption. LNPs can comprise a combination of different classes of lipids such as cationic or ionizable lipids (CIL), structural lipids (e.g., phospholipid and sterol lipid) and PEG-conjugated lipid (PEG-lipid). These lipids can self-assemble into LNPs under controlled microfluidic mixing with an aqueous phase containing the nucleic acids. PEG-lipids can prevent or reduce aggregation, degradation, and opsonization of the LNPs, while the structural lipids promote the stability and integrity of the nanoparticle.

In some embodiments, an LNP comprises the ionizable lipid DLin-MC3-DMA (MC3). In some embodiments, an LNP comprises DLin-MC3-DMA/DSPC/Cholesterol/PEG-lipid with the molar ratio 50:10:38.5:1.5. In some embodiments a delivery vector is not an LNP.

In some embodiments, a lipid-based delivery vector is or comprises a liposome. A liposome can comprise a cationic lipid, such as a cationic lipid disclosed herein. Illustrative liposomes include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV).

In some instances, liposomes provided herein also comprise carrier lipids. In some embodiments the carrier lipids are phospholipids. The carrier lipids are optionally any non-phosphate polar lipids. In some instances, liposomes provided herein comprise dipalmitoylphosphatidylcholine (DPPC), phosphatidylcholine (PC; lecithin), phosphatidic acid (PA), phosphatidylglycerol (PG), phosphatidylethanolamine (PE), phosphatidylserine (PS), distearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE) and the like, or a combination thereof. In some embodiments, the liposomes further comprise a sterol (e.g., cholesterol) which modulates liposome formation. In some embodiments, a liposome comprises an electroneutral lipid.

In some embodiments, a liposome comprises a cationic lipid. Cationic lipids can have a head group with positive charge (e.g., permanent or substantially permanent positive charge). Non-limiting examples of cationic lipids for use in liposomes include 1,2-di-O-octadecenyl-3-trimethylammonium-propane (DOTMA), 1,2-dioleoyl-3-trimethylammonium-propane (DOTAP), Dimethyldioctadecylammonium bromide (DDAB), and 2,3-dioleyloxy-N-[2-(sperminecarboxamido)ethyl]-N,N-dimethyl-1-propanaminium trifluoroacetate (DOSPA). In some embodiments a delivery vector is not a liposome.

In some embodiments, a lipid-based delivery vector is or comprises a vesicle, micelle or a microsphere. In some embodiments a delivery vector is not a vesicle, a micelle, or a microsphere.

A lipid-based delivery vector can be or comprise a micelle. In some instances, the micelle is a polymeric micelle, characterized by a core shell structure, in which the hydrophobic core is surrounded by a hydrophilic shell. In some cases, the hydrophilic shell further comprises a hydrophilic polymer or copolymer and a pH sensitive component.

Illustrative hydrophilic polymers or copolymers include, but are not limited to, poly(N-substituted acrylamides), poly(N-acryloyl pyrrolidine), poly(N-acryloyl piperidine), poly(N-acryl-L-amino acid amides), poly(ethyl oxazoline), methylcellulose, hydroxypropyl acrylate, hydroxyalkyl cellulose derivatives and poly(vinyl alcohol), poly(N-isopropylacrylamide), poly(N-vinyl-2-pyrrolidone), polyethyleneglycol derivatives, and combinations thereof.

A delivery vector can be or comprise a polymeric micelle exhibiting pH-sensitive properties, e.g., formed by using pH-sensitive polymers including, but not limited to, copolymers from methacrylic acid, methacrylic acid esters and acrylic acid esters, polyvinyl acetate phthalate, hydroxypropyl methyl cellulose phthalate, cellulose acetate phthalate, or cellulose acetate trimellitate.

A delivery vector can comprise a pH-sensitive moiety, which can include, but is not limited to, an alkylacrylic acid such as methacrylic acid, ethylacrylic acid, propyl acrylic acid and butyl acrylic acid, or an amino acid such as glutamic acid.

A delivery vector disclosed herein can be a non-viral vector. In some embodiments, a non-viral vector allows for superior delivery of a polynucleotide construct or polynucleotide upon repeat dosing compared to a viral vector, for example, based on reduced immunogenicity.

A delivery vector disclosed herein can be a non-viral, lipid-based delivery vector. A non-viral, lipid-based delivery vector can be, for example, an LDV disclosed herein, a liposome, a lipoplex, a lipid nanoparticle, a vesicle, or a micelle.

In some embodiments, a delivery vector is or comprises a poloxamer, nanoparticle, polyplex, or dendrimer.

A delivery vector can be a nanoparticle, for example, an inorganic nanoparticle, such as a gold, silica, iron oxide, titanium, calcium phosphate, PLGA, poly(ß-amino ester) (PBAE, e.g., PBAE-447), or hydrogel nanoparticle. In some embodiments a delivery vector is not a nanoparticle, e.g., is not an inorganic nanoparticle.

Nucleic acids can be encapsulated in particles through electrostatic association and physical entrapment. To prevent or slow the disassociation of cargo nucleic acids from nanoparticles following systemic administration, a polymerizable conjugate with a degradable, disulfide linkage can be employed. Nanoparticles can be encapsulated with a lipid coating to improve oral bioavailability, minimize enzymatic degradation and cross blood brain barrier. The nanoparticle surface can also be PEGylated to improve water solubility, circulation in vivo, and stealth properties.

A delivery vector can be a polyplex, for example, a complex of one or more polymers and nucleic acids. A polyplex can comprise cationic polymers. Fabrication of a polyplex can be based on self-assembly by ionic interactions. A polyplex can comprise polyethyleneimine, chitosan, poly(beta-amino esters), and/or polyphosphoramidate. In some embodiments a delivery vector is not a polyplex.

A delivery vector can be a dendrimer. A dendrimer can be a highly branched macromolecule with a spherical shape. The surface of dendrimer particles can be functionalized such as, for example, with positive surface charges (cationic dendrimers), which can be employed for the delivery of nucleic acids. Dendrimer-nucleic acid complexes are taken into a cell via endocytosis. In some embodiments a delivery vector is not a dendrimer.

In some embodiments, a delivery vector is or comprises a viral vector, a gamma-retroviral vector, a lentiviral vector, an adenoviral vector, or an adeno-associated viral vector. In some embodiments, a delivery vector is not a viral vector. In some embodiments, a delivery vector is not a retroviral vector. In some embodiments, a delivery vector is not a lentiviral vector. In some embodiments, a delivery vector is not an adenoviral vector. In some embodiments, a delivery vector is not an adeno-associated viral vector.

In some embodiments, a delivery vector is untargeted or is formulated for non-targeted delivery, for example, can facilitate delivery of a polynucleotide construct to a range of cell types including target cells and non-target cells (e.g., adipocytes and non-adipocytes cells, or white adipocytes and control cells that are not white adipocytes). A delivery vector can be capable of or configured for untargeted delivery. Specificity of expression in target cells upon non-targeted delivery can be facilitated by an expression regulatory region, such as a cell type-specific promoter.

In some embodiments, a delivery vector is targeted or is formulated for targeted delivery, and for example, can facilitate preferential delivery of a polynucleotide construct to a target cell type or population, such as adipocytes or white adipocytes. A delivery vector can be targeted to a receptor specifically or preferentially expressed on a target cell, for example, an adipocyte-specific receptor or surface protein, or a receptor or surface protein that exhibits higher expression on adipocytes as compared to control cells. Specificity of expression in target cells upon targeted delivery can be further enhanced by an expression regulatory region, such as a cell type-specific promoter.

A delivery vector disclosed herein can exclude a cell. For example, in some embodiments a delivery vector that is administered to a living subject does not include a cell, rather it comprises a polynucleotide that is delivered to the cell after administration (e.g., parenteral administration) of the delivery vector to the living subject.

### IV. SYSTEMS

In some embodiments, the present disclosure provides systems comprising a delivery vector and a polynucleotide construct for achieving a target cell specific reduction in the growth and/or survival of the target cell. Systems disclosed herein can find utility in a broad range of therapeutic applications in which it is desirable to modulate the growth or survival characteristics of an adipocyte, and to minimally reduce or substantially not reduce the growth or survival characteristics of a control cell, e.g., a non-adipocyte.

A system disclosed herein (e.g., for in vivo delivery of a polynucleotide construct encoding a cytotoxic protein) can comprise a delivery vector disclosed herein and a polynucleotide construct disclosed herein. The delivery vector can be, for example, a non-viral vector, a lipid-based delivery vector (LDV), or a non-viral LDV disclosed herein. The polynucleotide construct can comprise a transcriptional promoter that is selectively or preferentially active in a target cell (e.g., adipocyte or white adipocyte) driving expression of a transgene that encodes a therapeutic protein, such as a cytotoxic protein. Production and/or activation of the cytotoxic protein specifically or preferentially in adipocytes can provide a strategy for selective killing or reduction of adipocytes without the use of exogenously administered toxins. In some embodiments production and/or activation of the cytotoxic protein specifically or preferentially in adipocytes can provide a strategy for selective killing or reduction of adipocytes without target cell-specific delivery of the polynucleotide construct. Selectivity or preferential expression can utilize a combination of a promoter disclosed herein in conjunction with transcription-regulatory machinery that is provided by the target cell.

A system disclosed herein can further comprise an inducing agent. For example, the system can comprise: (i) a delivery vector disclosed herein, (ii) a polynucleotide construct that encodes an inducible cytotoxic protein (e.g., a rapamycin inducible caspase), and (iii) an inducing agent (e.g., rapamycin or a structural analogue thereof). The inducing agent or the rapamycin or structural analogue thereof can be or can comprise FK506, C-20-methyllyrlrapamycin (MaRap), C16(S)-Butylsulfonamidorapamycin (C16-BS-Rap), C16-(S)-7-methylindolerapamycin (AP21976/C16-AiRap), C16-(S)-3-mehylindolerapamycin (C16-iRap), Sirolimus, Tacrolimus, Everolimus, Temsirolimus, or Deforolimus.

A system can comprise (i) an LDV disclosed herein, (ii) a polynucleotide that encodes a rapamycin-inducible cytotoxic protein, and (iii) rapamycin or a structural analogue thereof.

A system disclosed herein can further comprise, e.g., one or more safety elements disclosed herein.

A system disclosed herein can comprise, for example, (i) a safety element, (ii) a polynucleotide construct that encodes an inducible cytotoxic protein (e.g., a rapamycin inducible caspase), (iii) an inducing agent (e.g., rapamycin or a structural analogue thereof), and/or (iv) a delivery vector disclosed herein.

A system disclosed herein can further comprise, an extracellular matrix remodeling factor. A system disclosed herein can comprise, for example, (i) an extracellular matrix remodeling factor, (ii) a polynucleotide construct that encodes an inducible cytotoxic protein (e.g., a rapamycin inducible caspase), (iii) an inducing agent (e.g., rapamycin or a structural analogue thereof), and/or (iv) a delivery vector disclosed herein.

### V. METHODS

Disclosed herein, in some aspects, is a method for reducing survival or persistence of a target cell (e.g., adipocyte or white adipocyte), the method comprising contacting the target cell with a polynucleotide construct, delivery vector, and/or system disclosed herein.

Disclosed herein, in some aspects, is a method for reducing growth of a target cell (e.g., adipocyte or white adipocyte), the method comprising contacting the target cell with a polynucleotide construct, delivery vector, inducing agent, and/or system disclosed herein. The contacting can be in vitro. The contacting can be in vivo. The polynucleotide construct can encode an inducible cytotoxic protein (e.g., rapamycin-inducible caspase), and the method can comprise contacting the cell with an inducing agent (e.g., rapamycin or a structural analogue thereof).

In some embodiments, the target cell is contacted with the polynucleotide construct, delivery vector, inducing agent, and/or system at a concentration of less than about 100 mM, less than about 10 mM, less than about 1 mM, less than about 500 μM, less than about 100 μM, less than about 50 μM, less than about 10 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, less than about 900 pM, less than about 800 pM, less than about 700 pM, less than about 600 pM, less than about 500 pM, less than about 400 pM, less than about 300 pM, less than about 200 pM, less than about 100 pM, less than about 10 pM, or less than about 1 pM.

In some embodiments, the target cell is contacted with the polynucleotide construct, delivery vector, inducing agent, and/or system at a concentration of at least about 100 mM, at least about 10 mM, at least about 1 mM, at least about 500 μM, at least about 100 μM, at least about 50 μM, at least about 10 μM, at least about 5 μM, at least about 4 μM, at least about 3 μM, at least about 2 μM, at least about 1 μM, at least about 900 nM, at least about 800 nM, at least about 700 nM, at least about 600 nM, at least about 500 nM, at least about 400 nM, at least about 300 nM, at least about 200 nM, at least about 100 nM, at least about 90 nM, at least about 80 nM, at least about 70 nM, at least about 60 nM, at least about 50 nM, at least about 40 nM, at least about 30 nM, at least about 20 nM, at least about 10 nM, at least about 5 nM, at least about 4 nM, at least about 3 nM, at least about 2 nM, at least about 1 nM, at least about 900 pM, at least about 800 pM, at least about 700 pM, at least about 600 pM, at least about 500 pM, at least about 400 pM, at least about 300 pM, at least about 200 pM, at least about 100 pM, at least about 10 pM, or at least about 1 pM.

In some embodiments, after the contacting, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, at least 95%, at least 97%, or at least 99% of adipocytes or white adipocytes are killed. Killing can be determined by a cytotoxicity assay, for example, an LDH release assay, a dye exclusion assay, flow cytometric evaluation, or other suitable methods.

In some embodiments, after the contacting, at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, or at most 90%, at most 95%, at most 97%, or at most 99% of adipocytes or white adipocytes are killed.

In some embodiments, after the contacting, about 1-90%, about 1-80%, about 1-70%, about 1-60%, about 1-50%, about 1-40%, about 1-30%, about 1-20%, about 1-10%, about 1-5%, about 5-90%, about 5-80%, about 5-70%, about 5-60%, about 5-50%, about 5-40%, about 5-30%, about 5-20%, about 5-10%, about 10-90%, about 10-80%, about 10-70%, about 10-60%, about 10-50%, about 10-40%, about 10-30%, about 10-20%, about 20-90%, about 20-80%, about 20-70%, about 20-60%, about 20-50%, about 20-40%, about 20-30%, about 30-90%, about 30-80%, about 30-70%, about 30-60%, about 30-50%, about 30-40%, about 40-90%, about 40-80%, about 40-70%, about 40-60%, about 40-50%, about 50-90%, about 50-80%, about 50-70%, about 50-60%, about 60-90%, about 60-80%, about 60-70%, about 70-90%, about 70-80%, or about 80-90% of the adipocytes or white adipocytes are killed.

A polynucleotide construct or system disclosed herein can exhibit preferential killing of adipocytes or white adipocytes over control cells, e.g., non-adipocytes or cells that are not white adipocytes. The control cells can be, e.g., myocytes, hepatocytes, osteocytes, erythrocytes, neurons, leukocytes, lymphocytes, monocytes, or fibroblasts, epithelial cells, or a combination thereof. In some embodiments the control cells are brown adipocytes. In some embodiments, upon contacting one or more populations of cells that comprise the adipocytes and/or control cells with the polynucleotide construct or system, killing (e.g., apoptosis) of the adipocytes is higher than killing of the control cells by a factor of at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 250 fold, at least 500 fold, or at least 1000 fold.

A method of the disclosure can comprise administering a polynucleotide, system, delivery vector, inducing agent, and/or pharmaceutical composition disclosed herein to a subject. The subject can be a mammal. The subject can be a human.

A polynucleotide construct, system, delivery vector, inducing agent, and/or pharmaceutical composition can be administered to an individual subject, for example, by parenteral administration, oral administration, or topical application. The administering can be local. The administering can be systemic. A polynucleotide construct, system, delivery vector, inducing agent, and/or pharmaceutical composition can be administered to an individual subject, for example, by intravenous, intraperitoneal, intramuscular, subdermal, intracerebral, intracerebroventricular, intra-articular, intraarterial, intrathecal, intracapsular, subcapsular, intraorbital, intracardiac, intradermal, subcutaneous, subarachnoid, or intracranial administration, e.g., injection or infusion. The administration can be via localized injection or infusion. The administration can be via systemic injection or infusion. The administration can be via intravenous injection or infusion. The administration can be via subcutaneous injection or infusion. The administration can be via local injection into adipose tissue, e.g., in a cavity or anatomical region disclosed herein. The administration can be via intraperitoneal injection.

Various dosing schedules can be used. In some embodiments, a polynucleotide construct, polynucleotide, system, delivery vector, inducing agent, and/or pharmaceutical composition is administered to a subject once. In some embodiments, a polynucleotide construct, polynucleotide, system, delivery vector, inducing agent, and/or pharmaceutical composition is administered to a subject two or more times. In some embodiments, a system disclosed herein comprising a non-integrating polynucleotide construct can allow for repeat administration with reduced likelihood of toxicity, for example, based on a limited number of copies of a polynucleotide construct that are degraded over time and/or become diluted as cells expressing the cytotoxic protein undergo cell death. A transgene can be expressed in a target cell (e.g., adipocyte) without genomic integration. For example, a transgene can be expressed from an eipsomal vector, such as a DNA, RNA, circular DNA, plasmid, circular RNA, minicircle, or the like. A transgene can be transiently expressed. For example, expression of a transgene can be reduced as a nucleic acid that encodes it is degraded.

Disclosed herein, in some aspects, is a method of reducing volume of an adipose tissue in a subject, the method comprising administering to the subject an effective amount of a polynucleotide construct, delivery vector, inducing agent, system, and/or pharmaceutical composition disclosed herein. The polynucleotide construct can encode an inducible cytotoxic protein (e.g., rapamycin-inducible caspase), and the method can comprise administering to the subject an inducing agent (e.g., rapamycin or a structural analogue thereof).

An inducing agent (e.g., rapamycin or a structural analogue thereof) can be administered by any suitable route of administration, for example, via a parenteral, oral, topical, local, systemic, subcutaneous, intravenous, intramuscular, intraperitoneal, subdermal, intracerebral, intracerebroventricular, intra-articular, intraarterial, intrathecal, intracapsular, subcapsular, intraorbital, intracardiac, intradermal, subcutaneous, subarachnoid, or intracranial route. The administration can be via localized injection or infusion. The administration can be via systemic injection or infusion. The administration can be via intravenous injection or infusion. The administration can be via subcutaneous injection or infusion. The administration can be via local injection into adipose tissue, e.g., in a cavity or anatomical region disclosed herein. The administration can be via intraperitoneal injection. The inducing agent can be administered by the same route as the polynucleotide construct or delivery vector. The inducing agent can be administered by a different route than the polynucleotide construct or delivery vector, for example, the polynucleotide construct or delivery vector can be administered via local parenteral administration, and the inducing agent can be administered orally.

In some embodiments, the inducing agent is administered locally, e.g., into an adipose tissue. In some embodiments, limited diffusion and/or a limited half-life of the inducing agent can advantageously limit activity of a cytotoxic protein disclosed herein outside of adipocytes, such as white adipocytes. In some embodiments, the inducing agent is administered systemically.

Adipose tissue can be reduced in a subject by a method disclosed herein. Adipose tissue can be reduced in, for example, visceral, subcutaneous, and/or abdominal region of a subject. Adipose tissue can be reduced in a single anatomical region or body cavity in a subject. Adipose tissue can be reduced in one or more specific regions or body cavities disclosed herein. For example, in some embodiments adipose tissue is reduced in subcutaneous fat. In some embodiments adipose tissue is reduced in visceral fat. In some embodiments, a promoter, expression regulatory region, or safety element disclosed herein contributes to specific or preferential reduction of adipose tissue on one or more specific regions or body cavities. In some embodiments, a route of administration disclosed herein contributes to specific or preferential reduction of adipose tissue on one or more specific regions or body cavities. Adipose tissue can be reduced in multiple anatomical regions or body cavities in a subject. A reduction in adipose tissue can be as determined by, for example, skinfold calipers, body circumference measurements, hydrostatic weighing, air displacement plethysmography, bioelectrical impedance analysis, bioimpedance spectroscopy, electrical impedance myography, multi-compartment models, an imaging technique, such as a DEXA scan, or a combination thereof, e.g., before and after treatment.

In some embodiments, a method disclosed herein reduces adipose tissue (e.g., white adipose tissue) in a subject (e.g., a in a particular anatomical region or body cavity, or globally) by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%.

In some embodiments, partial reduction of adipose tissue is advantageous over complete reduction in adipose tissue, for example, due to physiological processes mediated by adipose tissue in appropriate quantities. In some embodiments, a method disclosed herein reduces adipose tissue (e.g., white adipose tissue) in a subject (e.g., a in a particular anatomical region or body cavity, or globally) by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, or at most 90%.

In some embodiments, a method disclosed herein reduces adipose tissue (e.g., white adipose tissue) in a subject (e.g., a in a particular anatomical region or body cavity, or globally) by about 10-90%, about 10-80%, about 10-70%, about 10-60%, about 10-50%, about 10-40%, about 10-30%, about 10-20%, about 20-90%, about 20-80%, about 20-70%, about 20-60%, about 20-50%, about 20-40%, about 20-30%, about 30-90%, about 30-80%, about 30-70%, about 30-60%, about 30-50%, about 30-40%, about 40-90%, about 40-80%, about 40-70%, about 40-60%, about 40-50%, about 50-90%, about 50-80%, about 50-70%, about 50-60%, about 60-90%, about 60-80%, about 60-70%, about 70-90%, about 70-80%, or about 80-90%.

In some embodiments, brown adipose tissue is reduced by at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, or at most 90%.

In some embodiments, visceral adipose tissue (e.g., white visceral adipose tissue) is reduced in a subject.

In some embodiments, a method disclosed herein reduces visceral adipose tissue (e.g., white visceral adipose tissue) in a subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%.

In some embodiments, a method disclosed herein reduces visceral adipose tissue (e.g., white visceral adipose tissue) in a subject by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, or at most 90%.

In some embodiments, a method disclosed herein reduces visceral adipose tissue (e.g., white visceral adipose tissue) in a subject by about 10-90%, about 10-80%, about 10-70%, about 10-60%, about 10-50%, about 10-40%, about 10-30%, about 10-20%, about 20-90%, about 20-80%, about 20-70%, about 20-60%, about 20-50%, about 20-40%, about 20-30%, about 30-90%, about 30-80%, about 30-70%, about 30-60%, about 30-50%, about 30-40%, about 40-90%, about 40-80%, about 40-70%, about 40-60%, about 40-50%, about 50-90%, about 50-80%, about 50-70%, about 50-60%, about 60-90%, about 60-80%, about 60-70%, about 70-90%, about 70-80%, or about 80-90%.

In some embodiments, brown visceral adipose tissue is reduced by at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, or at most 90%.

In some embodiments, subcutaneous adipose tissue (e.g., white subcutaneous adipose tissue) is reduced in a subject.

In some embodiments, a method disclosed herein reduces subcutaneous adipose tissue (e.g., white subcutaneous adipose tissue) in a subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%.

In some embodiments, a method disclosed herein reduces subcutaneous adipose tissue (e.g., white subcutaneous adipose tissue) in a subject by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, or at most 90%.

In some embodiments, a method disclosed herein reduces subcutaneous adipose tissue (e.g., white subcutaneous adipose tissue) in a subject by about 10-90%, about 10-80%, about 10-70%, about 10-60%, about 10-50%, about 10-40%, about 10-30%, about 10-20%, about 20-90%, about 20-80%, about 20-70%, about 20-60%, about 20-50%, about 20-40%, about 20-30%, about 30-90%, about 30-80%, about 30-70%, about 30-60%, about 30-50%, about 30-40%, about 40-90%, about 40-80%, about 40-70%, about 40-60%, about 40-50%, about 50-90%, about 50-80%, about 50-70%, about 50-60%, about 60-90%, about 60-80%, about 60-70%, about 70-90%, about 70-80%, or about 80-90%.

In some embodiments, brown subcutaneous adipose tissue is reduced by at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, or at most 90%.

In some embodiments, abdominal adipose tissue (e.g., white abdominal adipose tissue) is reduced in a subject.

In some embodiments, a method disclosed herein reduces abdominal adipose tissue (e.g., white abdominal adipose tissue) in a subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%.

In some embodiments, a method disclosed herein reduces abdominal adipose tissue (e.g., white abdominal adipose tissue) in a subject by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, or at most 90%.

In some embodiments, a method disclosed herein reduces abdominal adipose tissue (e.g., white abdominal adipose tissue) in a subject by about 10-90%, about 10-80%, about 10-70%, about 10-60%, about 10-50%, about 10-40%, about 10-30%, about 10-20%, about 20-90%, about 20-80%, about 20-70%, about 20-60%, about 20-50%, about 20-40%, about 20-30%, about 30-90%, about 30-80%, about 30-70%, about 30-60%, about 30-50%, about 30-40%, about 40-90%, about 40-80%, about 40-70%, about 40-60%, about 40-50%, about 50-90%, about 50-80%, about 50-70%, about 50-60%, about 60-90%, about 60-80%, about 60-70%, about 70-90%, about 70-80%, or about 80-90%.

In some embodiments, brown abdominal adipose tissue is reduced by at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, or at most 90%.

Disclosed herein, in some aspects, is a method for treating a condition in a subject in need thereof, the method comprising administering to the subject an effective amount of a polynucleotide construct, delivery vector, system, and/or pharmaceutical composition disclosed herein. The polynucleotide construct can encode an inducible cytotoxic protein (e.g., rapamycin-inducible caspase), and the method can comprise administering to the subject an inducing agent (e.g., rapamycin or a structural analogue thereof).

A condition to be treated can be, comprise, or be associated with a metabolic disorder. A condition can be, comprise, or be associated with a lipidemia. A condition can be, comprise, or be associated with a rare lipidemia disease. A condition can be, comprise, or be associated with a lipoma. A condition can be, comprise, or be associated with fat accumulation, e.g., excess fat accumulation. A condition can be, comprise, or be associated with obesity. A condition can be, comprise, or be associated with morbid obesity. A condition can be, comprise, or be associated with being overweight. A condition can be, comprise, or be associated with an enzymatic deficiency or aberration. A condition can be, comprise, or be associated with a cancer.

In some embodiments, a composition, system, or method disclosed herein is used to reduce the body mass index (BMI) of a subject. BMI can describe a subject's weight in kilograms divided by the square of height in meters. In some embodiments, a subject to be treated has a BMI of at least 20, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40. In some embodiments, the method reduces BMI by at least 1, at least 2, at least 3, at least 4, at least 5, at least 5, at least 7, at least 8, at least 9, or at least 10 units.

In some embodiments, the condition is, comprises, or is associated with lipedema. Lipedema is a disease characterized by abnormal fat deposits in the legs. Lipedema is a chronic medical condition that can be characterized by a symmetric buildup of adipose tissue (fat) limbs, particularly in the legs. Lipedema can cause pain, swelling, and easy bruising, can make routine activities difficult, and can be non-responsive to diet and exercise compared to other types of fat accumulation. The condition can be, for example, stage 1 lipedema, stage 2 lipedema, or stage 3 lipedema.

In some embodiments, the condition is, comprises, or is associated with Dercum's disease (adiposis dolorosa). Dercum's disease is a rare disease of unknown etiology characterized by painful subcutaneous adipose tissue deposits with variable localization over the body. The deposits occur histologically as lipomas and are associated with overweight or obese status and a variety of psychiatric disturbances (anxiety, depression, sleep disturbances). The lipomas or adipose lumps can be small and difficult to palpate, or in a larger nodule form, and can cause severe chronic pain.

In some embodiments, a polynucleotide construct, delivery vector, system, and/or pharmaceutical composition disclosed herein is useful in a method of cosmetic fat removal.

Treating can comprise a pharmaceutical or other intervention regimen for obtaining beneficial or desired results in a subject. Beneficial or desired results include but are not limited to a therapeutic benefit and/or a prophylactic benefit. A therapeutic benefit can refer to eradication or amelioration of signs or symptoms of an underlying disorder being treated. A therapeutic benefit can be achieved with reduction or amelioration of one or more of the physiological signs or symptoms associated with the underlying disorder such that an improvement is observed or can be detected in the subject. A prophylactic effect can include delaying, preventing, or reducing the appearance of a disease or condition, delaying, preventing, or reducing the onset of symptoms of a disease or condition, delaying, preventing, reducing, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

Illustrative methods of treatment can include administration of a polynucleotide construct, polynucleotide, system, inducing agent, and/or delivery vector disclosed herein, including as part of a pharmaceutical composition. The expression construct, polynucleotide, system, and/or delivery vector can be administered in an amount effective to treat or prevent a disease or condition.

“Treatment” (and grammatical variations thereof such as “treat” or “treating”) can refer to clinical intervention in an attempt to alter the natural course of the individual (subject) being treated, and can be performed either for prophylaxis or during the course of clinical pathology. Desirable effects of treatment can include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

Pharmaceutical compositions of the present disclosure can comprise a composition disclosed herein and a pharmaceutically acceptable excipient. A pharmaceutical composition can comprise, for example, (i) a polynucleotide construct disclosed herein, an inducing agent disclosed herein, and/or a delivery vector disclosed herein, and (ii) a pharmaceutically acceptable excipient. A pharmaceutical composition can be formulated, for example, for systemic, local, parenteral, intratumoral, intravenous, intraperitoneal, subcutaneous, transdermal, or intramuscular administration. These compositions can take the form of solutions, suspensions, emulsion, tablets, pills, capsules, powders, sustained-release formulations, and the like.

The systems, compositions (e.g., pharmaceutical compositions) and methods of the present disclosure can be tested in vitro, and/or in vivo for the desired therapeutic or prophylactic activity. For example, in vitro assays to demonstrate the therapeutic or prophylactic utility of a compound or pharmaceutical composition include the effect of a system on a cell line or a patient tissue sample. The effect on the cell line and/or tissue sample can be determined utilizing techniques including, but not limited to proliferation and apoptosis assays. In accordance with the present disclosure, in vitro assays that can be used to determine whether administration of a specific compound is indicated, include in vitro cell culture assays in which a patient tissue sample is grown in culture, and exposed to or otherwise administered a compound, and the effect of such compound upon the tissue sample is observed.

In some embodiments, a polynucleotide construct is integrated into the genome of a host cell. A polynucleotide construct can be inserted into the genome of a cell in a targeted manner (e.g., at one or more specific site), or an untargeted manner (e.g., at one or more non-specific sites). For targeted integration, a polynucleotide sequence to be inserted can be flanked by homology arms comprising sequences that are complementary to a genomic DNA sequence to be targeted for insertion (e.g., via homologous recombination and/or homology-directed repair). A double stranded break can be introduced at a target site in the genome, and the homology arms can promote insertion of the polynucleotide. In some cases, a polynucleotide can be excised from a vector (e.g., via a nuclease), and inserted into the genome of the cell. A polynucleotide can be inserted in a safe harbor locus. A variety of enzymes can catalyze insertion of foreign DNA into a host genome. Non-limiting examples of gene editing tools and techniques include CRISPR, TALEN, zinc finger nuclease (ZFN), meganuclease, Mega-TAL, and transposon-based systems.

### VI. EXAMPLES

### Example 1: Construct Design

Polynucleotide constructs are designed for use in a gene therapy approach to induce killing of adipocytes and reduction of fat tissue.

The polynucleotide constructs comprise, from 5′ to 3′: (i) a promoter and/or expression regulatory region disclosed herein, such as an ADIPOQ, FABP4, PLIN1, PPARγ, PPARγ1, PPARγ2, CD36, LPL, LEP, CIDEC, TUSC5, CIDEA, or LIPE promoter or a functional fragment thereof; and (ii) a transgene encoding a cytotoxic protein disclosed herein, such as a caspase (e.g., an inducible caspase 9, such as a rapamycin-inducible caspase 9). Expression of the transgene is driven by the promoter and/or expression regulatory region. Certain polynucleotide constructs further encode one or more extracellular matrix remodeling factors and/or a safety elements (e.g., regulatory RNAs or regulatory RNA target sites).

The expression cassettes are cloned into NTC Nanoplasmids. Control constructs are generated in which the same promoters and regulatory regions drive expression of reporter genes (e.g., eGFP, luciferase, or E2-Crimson). Additional controls utilize constitutive or control promoters, e.g., CMV promoter.

### Example 2: Generation of LDV Formulations

For in vitro purposes LDVs comprising FAST proteins are hand mixed with various lipid formulations (e.g., as disclosed herein), and tested to evaluate transfection efficiency.

For in vivo purposes FAST-LDVs are manufactured to a final concentration of 2 mg/mL in PBS. LDVs are concentrated using a 100 kDa Ultra filter (Amicon, UFC810096) according to the manufacturer's instructions. LDVs are filter sterilized through 0.2 μm Acrodisc Supor filters (Amicon, UFC910008). Particle size, polydispersity index (PDI), and zeta potential are measured on final samples using the Malvern Zetasizer Range and a Universal ‘Dip’ Cell Kit (Malvern, ZEN1002) following the manufacturer's instructions. The nucleic acid encapsulation efficiency is calculated using a modified Quant-IT PicoGreen dsDNA assay (Thermo Fisher Scientific).

### Example 3: In Vitro Evaluation of Adipocyte Transfection, Adipocyte-Specific Gene Expression, and Adipocyte-Specific Killing

Human adipose-derived stromal/stem cells (Obatala catalog #OS-101) are cultured in Obatala Sciences StromaQual stromal medium until at least 80% confluence. Adipogenesis is induced using AdipoQual differentiation medium (Obatala catalog #OS-002).

The adipocytes are transfected in vitro using LDVs comprising FAST proteins disclosed herein, with 500-2000 ng of pDNA encapsulated in LDVs for 96-well plate (300 μl cell culture media final) and 250-1000 ng for 48-well plates (1000 μl cell culture media final).

To test transfection efficiency, reporter constructs (e.g., with eGFP driven by an adipocyte-specific (e.g., adiponectin) promoter/expression regulatory region or CMV control promoter) are transfected using LDV formulations disclosed herein. Transfected cells are imaged for eGFP expression over a period of 10 days. Transfected cells are also analyzed by flow cytometry to determine the percentage of eGFP positive cells. Obatala Sciences ObaFlow (Catalog #OS-304) is used for Adipocytes sample preparation. Reporter gene expression is quantified.

The ability of polynucleotide constructs, transcriptional promoters, and/or expression regulatory regions to induce specific or preferential expression in target cells is tested. Adipocytes, control cells, and/or co-cultures thereof are transfected with plasmids utilizing transcriptional promoters or expression regulatory regions disclosed herein driving expression of reporter genes. The percent of cells expressing the reporter genes is determined (e.g., via flow cytometry. Constructs with constitutive promoters are used as controls. Expression by target cells (e.g., adipocytes) is compared to expression by control cells.

The ability of polynucleotide constructs to induce killing of specific target cells (e.g., apoptosis of white adipocytes) is tested. Adipocytes, control cells, and/or co-cultures thereof are transfected with plasmids utilizing transcriptional promoters or expression regulatory regions preferentially or specifically active in human adipocytes to drive expression of cytotoxic proteins (e.g., iCasp9). Constructs with constitutive promoters are used as controls. After treatment, incubation, and addition of a chemical inducer of dimerization for inducible caspases, assays are conducted to determine viability of the adipocytes and control cells, for example, via flow cytometry (e.g., with annexin V or propidium iodide), LDH release, incucyte, or other viability/cytotoxicity assays. Killing of target cells (e.g., adipocytes) is compared to killing of control cells.

### Example 4: Reduction of Fat Volume in Human Explants

Human tissue explants were obtained from abdominoplasty (“tummy tuck”) surgical procedures via a commercial vendor (e.g., HypoSkin® from GenoSkin, comprising epidermis, dermis, and hypodermis with normal fat/subcutaneous tissue architecture). The explants were maintained in GenoSkin culture medium, and treated with 12 μg LDV formulations comprising polynucleotide constructs expressing inducible caspase 9.

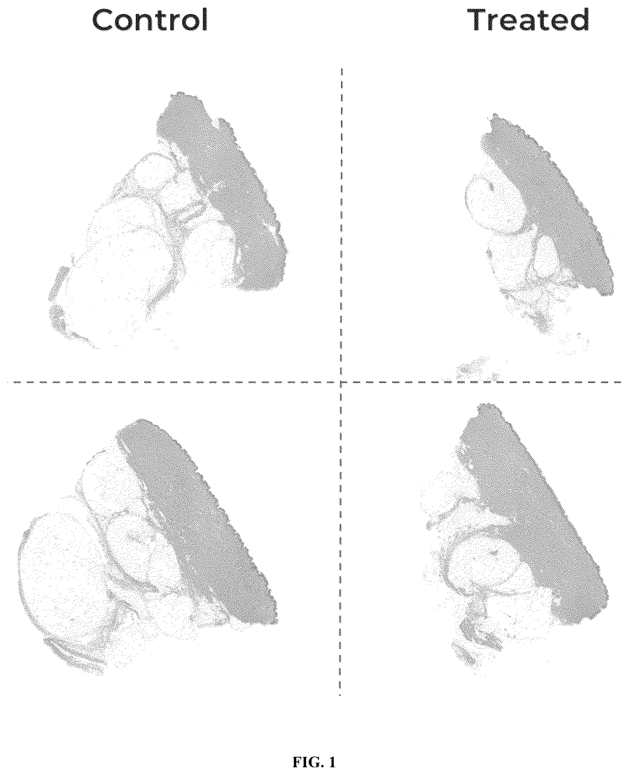

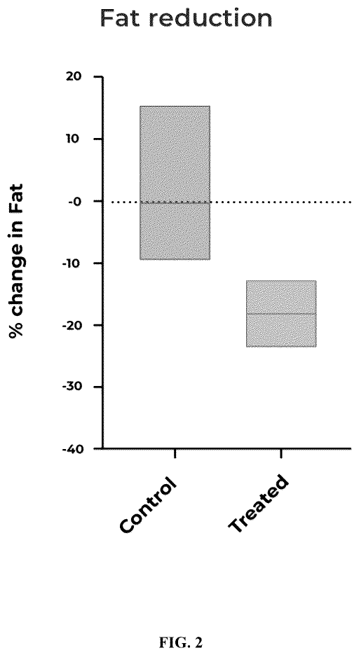

72 hours post-treatment, a chemical inducer of dimerization (CID) was added to the culture media to activate iCasp9. 24 hours after treatment with the CID, explants were fixed and processed for H&E staining (FIG. 1). Sections were imaged and analyzed to determine the percentage of tissue area that was adipose positive (mm²). Treatment led to an approximately 20% reduction in fat compared to control (FIG. 2).

### Example 5: Adipocyte-Specific Transgene Expression In Vivo

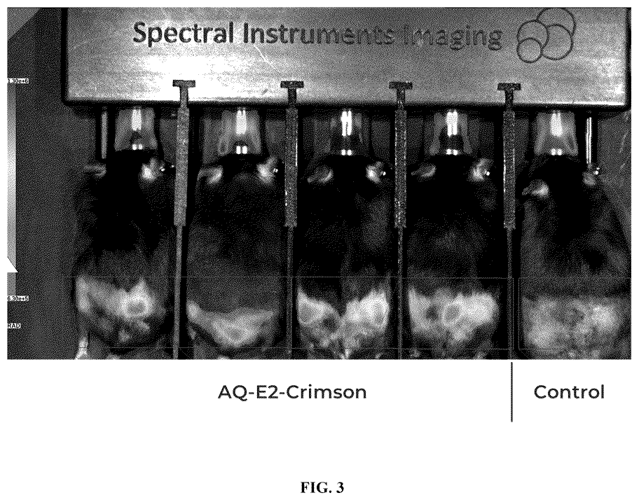

In vivo studies were done using adult female dB/dB mice (Jackson Lab). Mice received a subcutaneous injection of 100 μL of the test agent (e.g., 200 μg of plasmid expressing reporter E2-Crimson driven by adiponectin promoter; “AQ-E2-Crimson”), or PBS control. 72 hours after treatment, mice were imaged (e.g., via IVIS), showing that compositions and methods disclosed herein can be used to drive adipocyte-specific expression in vivo (FIG. 3).

### Example 6: Transgene Expression In Vivo

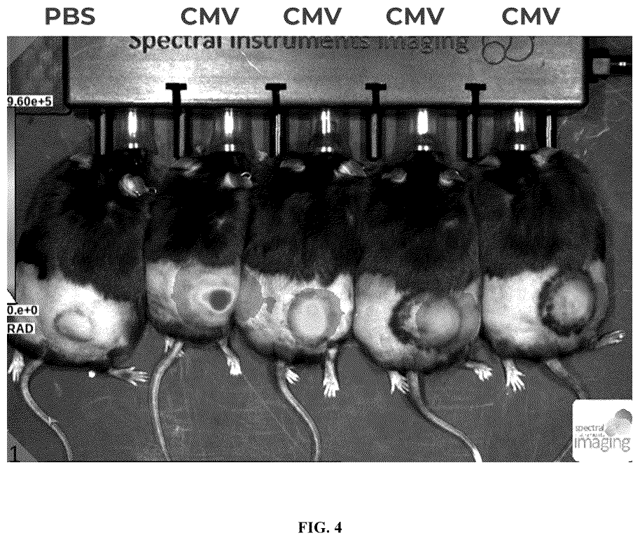

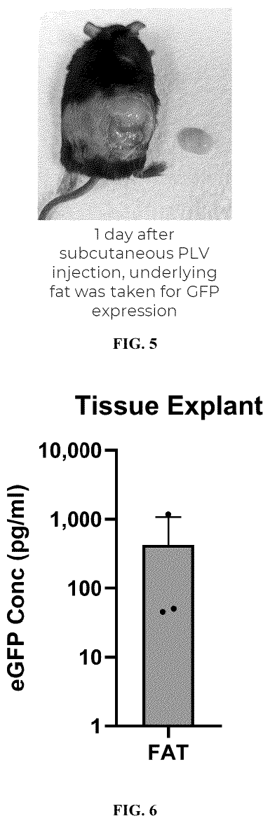

In vivo studies were done using adult female dB/dB mice (Jackson Lab). Five month old mice received a subcutaneous injection of 100 μL of the test agent (e.g., 200 μg LDVs containing plasmids expressing a 1:1 combination of eGFP and luciferase each driven by a CMV promoter) or PBS control. 24 hours after injection, animals were injected i.p. with D-luciferin and imaged using an AMI HT in vivo imager (FIG. 4). Afterwards, skin and fat pat samples near the injection site were collected (FIG. 5). Samples were lysed and a sandwich ELISA using Meso Scale Discovery (MSD) used to measure transgene expression, showing that eGFP was expressed (FIG. 6). The results show that administration of polynucleotide constructs can result in local expression of a transgene.

### Example 7: Fat Reduction In Vivo

In vivo studies are done using adult dB/dB mice. Five month old mice receive a subcutaneous injection of 100 μL of PBS control, or the test agent, for example, 200 μg LDVs containing plasmids with cytotoxic protein (e.g., iCasp9) expression driven by a promoter/expression regulatory region that induces specific or preferential expression in adipocytes. Subsequently, a chemical inducer of dimerization (e.g., rapamycin) is administered to the animals via intraperitoneal injection.

The effect of the polynucleotide construct on adipose tissue volume is quantified or monitored over time, for example, via DEXA scans, body weight measurements, body circumference measurements, hydrostatic weighing, air displacement plethysmography, bioelectrical impedance analysis, bioimpedance spectroscopy, electrical impedance myography, or a combination thereof.

### VII. ADDITIONAL SEQUENCES

TABLE 6

| SEQ | | |
|---|---|---|
| ID | | |
| NO | Description | Sequence |
| 2 | iCaspase9.r | ATGGCCAGCAGAATCCTGTGGCACGAGATGTGGCACGAG |
| | | GGCCTGGAGGAGGCCAGCAGACTGTACTTCGGCGAGAGA |
| | | AACGTGAAGGGCATGTTCGAGGTGCTGGAGCCCCTGCACG |
| | | CCATGATGGAGAGAGGCCCCCAGACCCTGAAGGAGACCA |
| | | GCTTCAACCAGGCCTACGGCAGAGACCTGATGGAGGCCCA |
| | | GGAGTGGTGCAGAAAGTACATGAAGAGCGGCAACGTGAA |
| | | GGACCTGCTGCAGGCCTGGGACCTGTACTACCACGTGTTC |
| | | AGAAGAATCAGCAAGCTGGAGTACAGCGGCGGCGGCAGC |
| | | CTGGAGGGCGTGCAGGTGGAGACCATCAGCCCCGGCGAC |
| | | GGCAGAACCTTCCCCAAGAGAGGCCAGACCTGCGTGGTGC |
| | | ACTACACCGGCATGCTGGAGGACGGCAAGAAGTTCGACA |
| | | GCAGCAGAGACAGAAACAAGCCCTTCAAGTTCATGCTGGG |
| | | CAAGCAGGAGGTGATCAGAGGCTGGGAGGAGGGCGTGGC |
| | | CCAGATGAGCGTGGGCCAGAGAGCCAAGCTGACCATCAG |
| | | CCCCGACTACGCCTACGGCGCCACCGGCCACCCCGGCATC |
| | | ATCCCCCCCCACGCCACCCTGGTGTTCGACGTGGAGCTGC |
| | | TGAAGCTGGAGAGCGGCGGCGGCGGCAGCGGCGGCGGCG |
| | | GCAGCGGCGGCGGCGGCAGCGGCGTGGACGGCTTCGGCG |
| | | ACGTGGGCGCCCTGGAGAGCCTGAGAGGCAACGCCGACC |
| | | TGGCCTACATCCTGAGCATGGAGCCCTGCGGCCACTGCCT |
| | | GATCATCAACAACGTGAACTTCTGCAGAGAGAGCGGCCTG |
| | | AGAACCAGAACCGGCAGCAACATCGACTGCGAGAAGCTG |
| | | AGAAGAAGATTCAGCAGCCTGCACTTCATGGTGGAGGTGA |
| | | AGGGCGACCTGACCGCCAAGAAGATGGTGCTGGCCCTGCT |
| | | GGAGCTGGCCCAGCAGGACCACGGCGCCCTGGACTGCTGC |
| | | GTGGTGGTGATCCTGAGCCACGGCTGCCAGGCCAGCCACC |
| | | TGCAGTTCCCCGGCGCCGTGTACGGCACCGACGGCTGCCC |
| | | CGTGAGCGTGGAGAAGATCGTGAACATCTTCAACGGCACC |
| | | AGCTGCCCCAGCCTGGGCGGCAAGCCCAAGCTGTTCTTCA |
| | | TCCAGGCCTGCGGCGGCGAGCAGAAGGACCACGGCTTCG |
| | | AGGTGGCCAGCACCAGCCCCGAGGACGAGAGCCCCGGCA |
| | | GCAACCCCGAGCCCGACGCCACCCCCTTCCAGGAGGGCCT |
| | | GAGAACCTTCGACCAGCTGGACGCCATCAGCAGCCTGCCC |
| | | ACCCCCAGCGACATCTTCGTGAGCTACAGCACCTTCCCCG |
| | | GCTTCGTGAGCTGGAGAGACCCCAAGAGCGGCAGCTGGTA |
| | | CGTGGAGACCCTGGACGACATCTTCGAGCAGTGGGCCCAC |
| | | AGCGAGGACCTGCAGAGCCTGCTGCTGAGAGTGGCCAAC |
| | | GCCGTGAGCGTGAAGGGCATCTACAAGCAGATGCCCGGCT |
| | | GCTTCAACTTCCTGAGAAAGAAGCTGTTCTTCAAGACCAG |
| | | CGCCAGCtaa |
| 14 | FRB-FKBP12- | ATGGCTTCTAGAATCCTCTGGCATGAGATGTGGCATGAAG |
| | L3-Casp9 fusion | GCCTGGAAGAGGCATCTCGTTTGTACTTTGGGGAAAGGAA |
| | protein | CGTGAAAGGCATGTTTGAGGTGCTGGAGCCCTTGCATGCT |
| | | ATGATGGAACGGGGCCCCCAGACTCTGAAGGAAACATCCT |
| | | TTAATCAGGCCTATGGTCGAGATTTAATGGAGGCCCAAGA |
| | | GTGGTGCAGGAAGTACATGAAATCAGGGAATGTCAAGGA |
| | | CCTCCTCCAAGCCTGGGACCTCTATTATCATGTGTTCCGAC |
| | | GAATCTCAAAGCTCGAGTATAGCGGCGGCGGCAGCCTGGA |
| | | GGGCGTGCAGGTGGAGACCATCAGCCCAGGCGACGGCAG |
| | | AACCTTCCCCAAGAGAGGCCAGACCTGCGTGGTGCACTAT |
| | | ACCGGCATGCTGGAGGACGGCAAGAAGTTCGACAGCAGC |
| | | CGCGACCGCAATAAGCCCTTCAAGTTCATGCTGGGCAAGC |
| | | AGGAGGTGATCAGAGGCTGGGAGGAGGGCGTGGCCCAGA |
| | | TGAGCGTGGGCCAGAGAGCCAAGCTGACCATCAGCCCCG |
| | | ACTACGCCTATGGCGCCACCGGCCACCCCGGCATCATCCC |
| | | ACCCCACGCCACCCTGGTGTTTGATGTGGAGCTGCTGAAG |
| | | CTGGAGTCCGGCGGAGGCGGGTCTGGAGGAGGCGGCAGC |
| | | GGCGGCGGCGGGTCAGGCGTGGATGGCTTCGGCGACGTG |
| | | GGAGCCCTGGAGAGCCTGAGAGGCAACGCCGATCTGGCCT |
| | | ACATCCTGAGCATGGAGCCCTGTGGCCACTGCCTGATCAT |
| | | CAACAACGTGAACTTCTGCCGGGAGAGCGGCCTGCGGACC |
| | | CGGACCGGCAGCAACATCGACTGCGAGAAGCTGAGGAGG |
| | | CGCTTCTCCTCCCTGCACTTTATGGTGGAGGTGAAAGGCG |
| | | ATCTGACTGCCAAGAAAATGGTGCTGGCCCTGCTGGAGCT |
| | | GGCCCAGCAGGACCACGGAGCCCTGGATTGCTGTGTGGTG |
| | | GTGATCCTGTCCCACGGCTGCCAGGCCAGCCACCTGCAGT |
| | | TCCCCGGAGCCGTGTACGGCACCGACGGCTGTCCCGTGTC |
| | | CGTGGAGAAGATCGTGAACATCTTCAACGGCACCTCCTGC |
| | | CCCTCCCTGGGCGGCAAGCCCAAGCTGTTCTTTATCCAGG |
| | | CCTGTGGCGGCGAGCAGAAGGACCACGGCTTTGAGGTGGC |
| | | CAGCACCTCCCCCGAGGACGAGAGCCCAGGCAGCAACCC |
| | | CGAGCCCGACGCCACCCCCTTCCAGGAGGGCCTGCGCACC |
| | | TTCGACCAGCTGGACGCCATCAGCAGCCTGCCCACCCCCA |
| | | GCGACATCTTCGTGAGCTACAGCACCTTTCCCGGCTTCGTG |
| | | AGCTGGCGCGATCCCAAGTCCGGCTCTTGGTATGTGGAGA |
| | | CCCTGGACGACATCTTTGAGCAGTGGGCTCATAGCGAGGA |
| | | CCTGCAGAGCCTGCTGCTGCGCGTGGCCAATGCCGTGAGC |
| | | GTGAAGGGCATCTACAAGCAGATGCCAGGCTGCTTCAACT |
| | | TCCTGCGGAAGAAGCTGTTCTTCAAGACCAGCGCCTCCTG |
| | | A |
| 15 | FKBP12-dCasp9 | ATGCTGGAGGGCGTGCAGGTGGAGACCATCAGCCCAGGC |
| | fusion protein | GACGGCAGAACCTTCCCCAAGAGAGGCCAGACCTGCGTG |
| | | GTGCACTATACCGGCATGCTGGAGGACGGCAAGAAGTTCG |
| | | ACAGCAGCCGCGACCGCAATAAGCCCTTCAAGTTCATGCT |
| | | GGGCAAGCAGGAGGTGATCAGAGGCTGGGAGGAGGGCGT |
| | | GGCCCAGATGAGCGTGGGCCAGAGAGCCAAGCTGACCAT |
| | | CAGCCCCGACTACGCCTATGGCGCCACCGGCCACCCCGGC |
| | | ATCATCCCACCCCACGCCACCCTGGTGTTTGATGTGGAGCT |
| | | GCTGAAGCTGGAGTCCGGAGGCGGCTCCGGCGTGGATGGC |
| | | TTCGGCGACGTGGGAGCCCTGGAGAGCCTGAGAGGCAAC |
| | | GCCGATCTGGCCTACATCCTGAGCATGGAGCCCTGTGGCC |
| | | ACTGCCTGATCATCAACAACGTGAACTTCTGCCGGGAGAG |
| | | CGGCCTGCGGACCCGGACCGGCAGCAACATCGACTGCGA |
| | | GAAGCTGAGGAGGCGCTTCTCCTCCCTGCACTTTATGGTG |
| | | GAGGTGAAAGGCGATCTGACTGCCAAGAAAATGGTGCTG |
| | | GCCCTGCTGGAGCTGGCCCAGCAGGACCACGGAGCCCTGG |
| | | ATTGCTGTGTGGTGGTGATCCTGTCCCACGGCTGCCAGGC |
| | | CAGCCACCTGCAGTTCCCCGGAGCCGTGTACGGCACCGAC |
| | | GGCTGTCCCGTGTCCGTGGAGAAGATCGTGAACATCTTCA |
| | | ACGGCACCTCCTGCCCCTCCCTGGGCGGCAAGCCCAAGCT |
| | | GTTCTTTATCCAGGCCTGTGGCGGCGAGCAGAAGGACCAC |
| | | GGCTTTGAGGTGGCCAGCACCTCCCCCGAGGACGAGAGCC |
| | | CAGGCAGCAACCCCGAGCCCGACGCCACCCCCTTCCAGGA |
| | | GGGCCTGCGCACCTTCGACCAGCTGGACGCCATCAGCAGC |
| | | CTGCCCACCCCCAGCGACATCTTCGTGAGCTACAGCACCT |
| | | TTCCCGGCTTCGTGAGCTGGCGCGATCCCAAGTCCGGCTCT |
| | | TGGTATGTGGAGACCCTGGACGACATCTTTGAGCAGTGGG |
| | | CTCATAGCGAGGACCTGCAGAGCCTGCTGCTGCGCGTGGC |
| | | CAATGCCGTGAGCGTGAAGGGCATCTACAAGCAGATGCCA |
| | | GGCTGCTTCAACTTCCTGCGGAAGAAGCTGTTCTTCAAGA |
| | | CCAGCGCCTCCTGA |
| 16 | FRB-dCasp9 | ATGGCTTCTAGAATCCTCTGGCATGAGATGTGGCATGAAG |
| | fusion protein | GCCTGGAAGAGGCATCTCGTTTGTACTTTGGGGAAAGGAA |
| | | CGTGAAAGGCATGTTTGAGGTGCTGGAGCCCTTGCATGCT |
| | | ATGATGGAACGGGGCCCCCAGACTCTGAAGGAAACATCCT |
| | | TTAATCAGGCCTATGGTCGAGATTTAATGGAGGCCCAAGA |
| | | GTGGTGCAGGAAGTACATGAAATCAGGGAATGTCAAGGA |
| | | CCTCCTCCAAGCCTGGGACCTCTATTATCATGTGTTCCGAC |
| | | GAATCTCAAAGCTCGAGTATAGCGGCGGCGGCAGCGGCGT |
| | | GGATGGCTTCGGCGACGTGGGAGCCCTGGAGAGCCTGAG |
| | | AGGCAACGCCGATCTGGCCTACATCCTGAGCATGGAGCCC |
| | | TGTGGCCACTGCCTGATCATCAACAACGTGAACTTCTGCC |
| | | GGGAGAGCGGCCTGCGGACCCGGACCGGCAGCAACATCG |
| | | ACTGCGAGAAGCTGAGGAGGCGCTTCTCCTCCCTGCACTT |
| | | TATGGTGGAGGTGAAAGGCGATCTGACTGCCAAGAAAAT |
| | | GGTGCTGGCCCTGCTGGAGCTGGCCCAGCAGGACCACGGA |
| | | GCCCTGGATTGCTGTGTGGTGGTGATCCTGTCCCACGGCTG |
| | | CCAGGCCAGCCACCTGCAGTTCCCCGGAGCCGTGTACGGC |
| | | ACCGACGGCTGTCCCGTGTCCGTGGAGAAGATCGTGAACA |
| | | TCTTCAACGGCACCTCCTGCCCCTCCCTGGGCGGCAAGCC |
| | | CAAGCTGTTCTTTATCCAGGCCTGTGGCGGCGAGCAGAAG |
| | | GACCACGGCTTTGAGGTGGCCAGCACCTCCCCCGAGGACG |
| | | AGAGCCCAGGCAGCAACCCCGAGCCCGACGCCACCCCCTT |
| | | CCAGGAGGGCCTGCGCACCTTCGACCAGCTGGACGCCATC |
| | | AGCAGCCTGCCCACCCCCAGCGACATCTTCGTGAGCTACA |
| | | GCACCTTTCCCGGCTTCGTGAGCTGGCGCGATCCCAAGTC |
| | | CGGCTCTTGGTATGTGGAGACCCTGGACGACATCTTTGAG |
| | | CAGTGGGCTCATAGCGAGGACCTGCAGAGCCTGCTGCTGC |
| | | GCGTGGCCAATGCCGTGAGCGTGAAGGGCATCTACAAGCA |
| | | GATGCCAGGCTGCTTCAACTTCCTGCGGAAGAAGCTGTTC |
| | | TTCAAGACCAGCGCCTCCTGA |
| 17 | FKBP12-Casp9- | ATGCTGGAGGGCGTGCAGGTGGAGACCATCAGCCCAGGC |
| | 2A-FRB-FRBw | GACGGCAGAACCTTCCCCAAGAGAGGCCAGACCTGCGTG |
| | | GTGCACTATACCGGCATGCTGGAGGACGGCAAGAAGTTCG |
| | | ACAGCAGCCGCGACCGCAATAAGCCCTTCAAGTTCATGCT |
| | | GGGCAAGCAGGAGGTGATCAGAGGCTGGGAGGAGGGCGT |
| | | GGCCCAGATGAGCGTGGGCCAGAGAGCCAAGCTGACCAT |
| | | CAGCCCCGACTACGCCTATGGCGCCACCGGCCACCCCGGC |
| | | ATCATCCCACCCCACGCCACCCTGGTGTTTGATGTGGAGCT |
| | | GCTGAAGCTGGAGTCCGGAGGCGGCTCCGGCGTGGATGGC |
| | | TTCGGCGACGTGGGAGCCCTGGAGAGCCTGAGAGGCAAC |
| | | GCCGATCTGGCCTACATCCTGAGCATGGAGCCCTGTGGCC |
| | | ACTGCCTGATCATCAACAACGTGAACTTCTGCCGGGAGAG |
| | | CGGCCTGCGGACCCGGACCGGCAGCAACATCGACTGCGA |
| | | GAAGCTGAGGAGGCGCTTCTCCTCCCTGCACTTTATGGTG |
| | | GAGGTGAAAGGCGATCTGACTGCCAAGAAAATGGTGCTG |
| | | GCCCTGCTGGAGCTGGCCCAGCAGGACCACGGAGCCCTGG |
| | | ATTGCTGTGTGGTGGTGATCCTGTCCCACGGCTGCCAGGC |
| | | CAGCCACCTGCAGTTCCCCGGAGCCGTGTACGGCACCGAC |
| | | GGCTGTCCCGTGTCCGTGGAGAAGATCGTGAACATCTTCA |
| | | ACGGCACCTCCTGCCCCTCCCTGGGCGGCAAGCCCAAGCT |
| | | GTTCTTTATCCAGGCCTGTGGCGGCGAGCAGAAGGACCAC |
| | | GGCTTTGAGGTGGCCAGCACCTCCCCCGAGGACGAGAGCC |
| | | CAGGCAGCAACCCCGAGCCCGACGCCACCCCCTTCCAGGA |
| | | GGGCCTGCGCACCTTCGACCAGCTGGACGCCATCAGCAGC |
| | | CTGCCCACCCCCAGCGACATCTTCGTGAGCTACAGCACCT |
| | | TTCCCGGCTTCGTGAGCTGGCGCGATCCCAAGTCCGGCTCT |
| | | TGGTATGTGGAGACCCTGGACGACATCTTTGAGCAGTGGG |
| | | CTCATAGCGAGGACCTGCAGAGCCTGCTGCTGCGCGTGGC |
| | | CAATGCCGTGAGCGTGAAGGGCATCTACAAGCAGATGCCA |
| | | GGCTGCTTCAACTTCCTGCGGAAGAAGCTGTTCTTCAAGA |
| | | CCAGCGCCTCCCAGTGCACCAATTATGCTTTGCTTAAGCTG |
| | | GCAGGCGATGTGGAATCAAACCCGGGTCCTGGGGTACAG |
| | | GTGGAGACCATCTCTCCTGGCGACGGGAGAACATTTCCTA |
| | | AAAGGGGCCAAACATGCGTGGTTCACTATACCGGTATGCT |
| | | GGAGGATGGCAAAAAAGTAGACTCCAGCCGGGATAGAAA |
| | | CAAACCCTTTAAGTTCATGCTGGGTAAGCAGGAAGTTATA |
| | | CGGGGCTGGGAAGAGGGAGTAGCTCAGATGTCTGTGGGC |
| | | CAGAGGGCCAAGCTGACCATCTCACCGGACTACGCCTACG |
| | | GCGCTACCGGCCACCCTGGCATTATACCACCCCATGCAAC |
| | | TCTCGTCTTCGATGTTGAGTTGCTCAAACTGGAATCAGGCG |
| | | GAGGCGGGTCTGGAGGAGGCGGCAGCATGCTGGAGGGCG |
| | | TGCAGGTGGAGACCATCAGCCCAGGCGACGGCAGAACCTT |
| | | CCCCAAGAGAGGCCAGACCTGCGTGGTGCACTATACCGGC |
| | | ATGCTGGAGGACGGCAAGAAGTTCGACAGCAGCCGCGAC |
| | | CGCAATAAGCCCTTCAAGTTCATGCTGGGCAAGCAGGAGG |
| | | TGATCAGAGGCTGGGAGGAGGGCGTGGCCCAGATGAGCG |
| | | TGGGCCAGAGAGCCAAGCTGACCATCAGCCCCGACTACGC |
| | | CTATGGCGCCACCGGCCACCCCGGCATCATCCCACCCCAC |
| | | GCCACCCTGGTGTTTGATGTGGAGCTGCTGAAGCTGGAGT |
| | | CCTGA |
| 23 | BGH PA (CpG | CTACTGTGCCTTCTAGTTGCCAGCCATCTGTTGTTTGCCCC |
| | free-long) | TCCCCCTTGCCTTCCTTGACCCTGGAAGGTGCCACTCCCAC |
| | | TGTCCTTTCCTAATAAAATGAGGAAATTGCATCACATTGTC |
| | | TGAGTAGGTGTCATTCTATTCTGGGGGGGGGGTGGGGCA |
| | | GGACAGCAAGGGGGAGGATTGGGAAGACAATAGCAGGCA |
| | | TGCTGGGGATGCAGTGGGCTCTATGG |
| 24 | Beta globin | AATAAAGGAAATTTATTTTCATTGCAATAGTGTGTTGGAA |
| | poly(A) signal | TTTTTTGTGTCTCTCA |
| 25 | Flexible linker/ | GGGGSGGGGSGGGGS |
| | linker subunit | |
| 26 | Flexible linker/ | GGGGS |
| | linker subunit | |
| 27 | Flexible linker/ | GGGS |
| | linker subunit | |
| 28 | Flexible linker/ | GG |
| | linker subunit | |
| 29 | Flexible linker/ | KESGSVSSEQLAQFRSLD |
| | linker subunit | |
| 30 | Flexible linker/ | EGKSSGSGSESKST |
| | linker subunit | |
| 31 | Flexible linker/ | GSAGSAAGSGEF |
| | linker subunit | |
| 32 | Rigid | EAAAK |
| | linker/linker | |
| | subunit | |
| 33 | Rigid | EAAAR |
| | linker/linker | |
| | subunit | |
| 34 | Rigid | PAPAP |
| | linker/linker | |
| | subunit | |
| 35 | Rigid | AEAAAKEAAAKA |
| | linker/linker | |
| | subunit | |
| 36 | F2A | APVKQTLNFDLLKLAGDVESNPGP |
| 37 | P2A | ATNFSLLKQAGDVEENPGP |
| 38 | E2A | QCTNYALLKLAGDVESNPGP |
| 39 | T2A | EGRGSLLTCGDVEENPGP |
| 40 | GSG-F2A | GSGAPVKQTLNFDLLKLAGDVESNPGP |
| 41 | GSG-P2A | GSGATNFSLLKQAGDVEENPGP |
| 42 | GSG-E2A | GSGQCTNYALLKLAGDVESNPGP |
| 43 | GSG-T2A | GSGEGRGSLLTCGDVEENPGP |
| 44 | Furin-F2A | RRKRAPVKQTLNFDLLKLAGDVESNPGP |
| 45 | Furin-P2A | RRKRATNFSLLKQAGDVEENPGP |
| 46 | Furin-E2A | RRKRQCTNYALLKLAGDVESNPGP |
| 47 | Furin-T2A | RRKREGRGSLLTCGDVEENPGP |
| 48 | Furin-GSG-F2A | RRKRGSGAPVKQTLNFDLLKLAGDVESNPGP |
| 49 | Furin-GSG-P2A | RRKRGSGATNFSLLKQAGDVEENPGP |
| 50 | Furin-GSG-E2A | RRKRGSGQCTNYALLKLAGDVESNPGP |
| 51 | Furin-GSG-T2A | RRKRGSGEGRGSLLTCGDVEENPGP |

The degree of sequence identity between two sequences can be determined, for example, by comparing the two sequences using computer programs designed for this purpose, such as global or local alignment algorithms. Non-limiting examples include BLASTp, BLASTn, Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, SSEARCH2SEQ, or another suitable method or algorithm. A global alignment algorithm, such as a Needleman and Wunsch algorithm, can be used to align two sequences over their entire length, maximizing the number of matches and minimizes the number of gaps. Default settings can be used.

To generate similarity scores for two amino acid sequences, scoring matrices can be used that assign positive scores for some non-identical amino acids (e.g., amino acids with similar physio-chemical properties and/or amino acids that exhibit frequent substitutions in orthologs, homologs, or paralogs), Non-limiting examples of scoring matrices include PAM30, PAM70, PAM250, BLOSUM45, BLOSUM50, BLOSUM62, BLOSUM80, and BLOSUM90.

Amino acids can include genetically encoded and non-genetically encoded occurring amino acids. Amino acids can include naturally occurring and non-naturally occurring amino acids. Amino acids can be L forms or D forms. Substitutions disclosed herein can include conservative and/or non-conservative amino acid substitutions. A conservative amino acid substitution can be a substitution of one amino acid for another amino acid of similar biochemical properties (e.g., charge, size, and/or hydrophobicity). A non-conservative amino acid substitution can be a substitution of one amino acid for another amino acid with different biochemical properties (e.g., charge, size, and/or hydrophobicity). A conservative amino acid change can be, for example, a substitution that has minimal effect on the secondary or tertiary structure of a polypeptide. A conservative amino acid change can be an amino acid change from one hydrophilic amino acid to another hydrophilic amino acid. Hydrophilic amino acids can include Thr (T), Ser(S), His (H), Glu (E), Asn (N), Gln (Q), Asp (D), Lys (K) and Arg (R). A conservative amino acid change can be an amino acid change from one hydrophobic amino acid to another hydrophilic amino acid. Hydrophobic amino acids can include Ile (I), Phe (F), Val (V), Leu (L), Trp (W), Met (M), Ala (A), Gly (G), Tyr (Y), and Pro (P). A conservative amino acid change can be an amino acid change from one acidic amino acid to another acidic amino acid. Acidic amino acids can include Glu (E) and Asp (D). A conservative amino acid change can be an amino acid change from one basic amino acid to another basic amino acid. Basic amino acids can include His (H), Arg (R) and Lys (K). A conservative amino acid change can be an amino acid change from one polar amino acid to another polar amino acid. Polar amino acids can include Asn (N), Gln (Q), Ser(S) and Thr (T). A conservative amino acid change can be an amino acid change from one nonpolar amino acid to another nonpolar amino acid. Nonpolar amino acids can include Leu (L), Val (V), Ile (I), Met (M), Gly (G) and Ala (A). A conservative amino acid change can be an amino acid change from one aromatic amino acid to another aromatic amino acid. Aromatic amino acids can include Phe (F), Tyr (Y) and Trp (W). A conservative amino acid change can be an amino acid change from one aliphatic amino acid to another aliphatic amino acid. Aliphatic amino acids can include Ala (A), Val (V), Leu (L) and Ile (I). In some embodiments, a conservative amino acid substitution is an amino acid change from one amino acid to another amino acid within one of the following groups: Group I: Ala, Pro, Gly, Gln, Asn, Ser, Thr, Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu.

A protein or polypeptide disclosed herein can comprise an N-terminal methionine. A protein or polypeptide disclosed herein can lack an N-terminal methionine.

1.-75. (canceled)

76. A polynucleotide construct for selective killing of human adipocytes, the polynucleotide construct comprising:
(a) a transcriptional promoter that is preferentially or specifically active in a human adipocyte; and
(b) a transgene encoding a cytotoxic protein, wherein expression of the cytotoxic protein is regulated by the transcriptional promoter.

77. The polynucleotide construct of claim 76, wherein the transcriptional promoter comprises an ADIPOQ promoter or a functional fragment thereof.

78. The polynucleotide construct of claim 76, wherein the transcriptional promoter comprises a FABP4, PLIN1, PPARγ, PPARγ1, PPARγ2, CD36, LPL, LEP, CIDEC, TUSC5, CIDEA, or LIPE promoter or a functional fragment thereof.

79. The polynucleotide construct of claim 76, wherein the transcriptional promoter comprises a nucleic acid sequence with at least about 80% sequence identity to any one of SEQ ID NOs: 1 and 56-72.

80. The polynucleotide construct of claim 76, wherein the cytotoxic protein induces non-inflammatory cell death upon expression of the cytotoxic protein in the human adipocyte.

81. The polynucleotide construct of claim 76, wherein the cytotoxic protein comprises a caspase or a catalytic domain thereof.

82. The polynucleotide construct of claim 81, wherein the caspase is a self-activating caspase.

83. The polynucleotide construct of claim 81, wherein the caspase comprises an inducible caspase or catalytic domain thereof.

84. The polynucleotide construct of claim 81, wherein the caspase comprises a rapamycin-inducible caspase.

85. The polynucleotide construct of claim 81, wherein the caspase comprises a caspase 9 or catalytic domain thereof.

86. The polynucleotide construct of claim 81, wherein the cytotoxic protein comprises an amino acid sequence with at least 80% sequence identity to any one of SEQ ID NOs: 3-13.

87. The polynucleotide construct of claim 76, wherein the human adipocyte is a white adipocyte.

88. The polynucleotide construct of claim 76, wherein the human adipocyte is a not a brown adipocyte.

89. A lipid-based delivery vector (LDV) comprising the polynucleotide construct of claim 76 and a fusion-associated small transmembrane (FAST) protein.

90. The LDV of claim 89, wherein the FAST protein comprises an ectodomain of p14 and an endodomain of p15.

91. A method of reducing viability of a population of adipocytes, the method comprising contacting the population of adipocytes with a lipid-based delivery vector (LDV) comprising a polynucleotide construct under conditions that facilitate uptake of the polynucleotide construct by the adipocytes, wherein the polynucleotide construct comprises a transgene and a transcriptional promoter that regulates expression of the transgene, wherein the transgene encodes a cytotoxic protein.

92. A method of reducing adipose tissue volume in a subject, the method comprising administering to the subject an effective amount of a lipid-based delivery vector (LDV) comprising a polynucleotide construct, wherein the polynucleotide construct comprises a transgene and a transcriptional promoter that regulates expression of the transgene, wherein the transgene encodes a cytotoxic protein.

93. The method of claim 92, wherein the adipose tissue volume is reduced by at least about 5%.

94. The method of claim 92, wherein the method treats a lipoma in the subject.

95. The method of claim 92, wherein the method treats Dercum's disease in the subject.
